(19) United States
(12) Patent Application Publication
(10) Pub. No.: US 2010/0286260 A1
(43) Pub. Date: Nov. 11, 2010

(54) MILNACIPRAN FORMULATIONS

(75) Inventors: Mahendra G. Dedhiya, Pomona, NY (US); Anil Chhettry, Holtsville, NY (US); Yan Yang, Roslyn Heights, NY (US); Yun Mo, Commack, NY (US); Bhaveshkumar H. Kothari, Smithtown, NY (US)

Correspondence Address: Forest Laboratories, Inc.;Attn: Charles S. Ryan, 500 COMMACK ROAD, Commack, NY, 11725, US

(73) Assignee: Forest Laboratories Holdings Ltd., Hamilton (BM)

(21) Appl. No.: 12/774,446

(22) Filed: May 5, 2010

Related U.S. Application Data
(60) Provisional application No. 61/175,674, filed on May 5, 2009.

(51) Int. Cl. A61K 31/365; A61P 1/08; A61P 25/22; A61P 13/00; A61P 1/12

(52) U.S. Cl. 514/470

(57) ABSTRACT

The present invention provides formulations comprising milnacipran or pharmaceutically acceptable salts thereof (e.g., milnacipran hydrochloride), including immediate release formulations and modified formulations, such as delayed release and extended release formulations. The present invention provides formulations with improved stability and high bioavailability. Processes for preparing the formulations as well as methods of treating conditions by administering the formulations are also described.

### CROSS-REFERENCE TO RELATED APPLICATIONS

This application claims priority under 35 U.S.C. §119, based on U.S. Provisional Application Ser. No. 61/175,674 filed on May 5, 2009, which is hereby incorporated by reference in its entirety.

### FIELD OF THE INVENTION

The present invention relates to formulations comprising milnacipran or pharmaceutically acceptable salts thereof (e.g., milnacipran hydrochloride), including immediate release formulations and modified formulations (e.g., delayed release or extended release formulations). The present invention provides formulations with improved stability and high bioavailability. Processes for preparing the formulations as well as methods of treating conditions by administering the formulations are also described.

### BACKGROUND OF THE INVENTION

Milnacipran is a norepinephrine-serotonin reuptake inhibitor (NSRI), which inhibits the uptake of both norepinephrine (NE) and serotonin (5-HT), with an NE to 5-HT ratio of 2:1 (See, e.g., Moret et al., Neuropharmacology, 24:1211-1219, 1985; Palmier et al., Eur. J. Clin. Pharmacol., 37:235-238, 1989) but does not affect the uptake of dopamine. Milnacipran and methods of treatment using milnacipran are disclosed, for example, in U.S. Pat. Nos. 4,478,836; 6,602,911; 6,635,675 and 6,992,110.

U.S. Pat. Nos. 4,478,836; 6,602,911; 6,635,675 and 6,992,110 are incorporated herein by reference, in their entirety.

Adverse events associated with immediate release formulations of milnacipran administration may include, for example, nausea, vomiting, headache, tremulousness, anxiety, panic attack, palpitations, urinary retention, orthostatic hypotension, diaphoresis, chest pain, rash, weight increase, back pain, constipation, diarrhea, vertigo, increased sweating, agitation, hot flushes, fatigue, somnolence, dyspepsia, dysuria, dry mouth, abdominal pain, and insomnia. Due to the incidence of adverse events, patients often do not tolerate high-doses of milnacipran.

There remains a need for improved formulations of milnacipran or pharmaceutically acceptable salts thereof, including formulations that have improved safety, stability and performance properties. The present invention seeks to provide such pharmaceutical formulations, as well as processes for making the formulations and methods of treating conditions by administering the formulations.

### SUMMARY OF THE INVENTION

The present invention provides pharmaceutical formulations comprising milnacipran or pharmaceutically acceptable salts thereof (e.g., milnacipran hydrochloride), including immediate release and modified release formulations (e.g., delayed release or extended release formulations).

According to some embodiments, the present invention provides formulations comprising about 12.5 mg to about 200 mg milnacipran or a pharmaceutically acceptable salt thereof; and about 0.03 to about 0.5% w/w 1-phenyl-3-aza-bicyclo[3.1.0]hexan-2-one that release more than about 80% milnacipran or pharmaceutically acceptable salt thereof within 30 minutes upon entry in a use environment.

According to other embodiments, the present invention provides formulations comprising about 12.5 mg to about 200 mg milnacipran or a pharmaceutically acceptable salt thereof; about 0.03 to about 0.5% w/w 1-phenyl-3-aza-bicyclo[3.1.0]hexan-2-one; and about 0.5 to about 8% w/w of a coating that comprises a polymer in combination with a surfactant and has an adhesion force of more than about 25 N.

According to still other embodiments, the present invention provides formulations comprising about 12.5 mg to about 200 mg milnacipran or a pharmaceutically acceptable salt thereof; about 0.03 to about 0.5% w/w 1-phenyl-3-aza-bicyclo[3.1.0]hexan-2-one; and about 0.5 to about 8% w/w of a coating that comprises a combination of a polymer and a surfactant in a ratio of between about to 1:1 and about 20:1.

According to some embodiments, the present invention provides extended release formulations comprising about 12.5 mg to about 300 mg milnacipran or a pharmaceutically acceptable salt thereof, and about 0.03 to about 0.5% w/w 1-phenyl-3-aza-bicyclo[3.1.0]hexan-2-one.

According to other embodiments, the present invention provides delayed release formulations comprising about 12.5 mg to about 300 mg milnacipran or a pharmaceutically acceptable salt thereof, and about 0.03 to about 0.5% w/w 1-phenyl-3-aza-bicyclo[3.1.0]hexan-2-one.

### BRIEF DESCRIPTION OF THE DRAWINGS

FIG. 1 shows the dissolution profile for hydroxypropyl methylcellulose (HPMC) coated milnacipran hydrochloride immediate release (IR) tablets.

FIG. 2 shows the dissolution profile for polyvinyl alcohol (PVA) coated milnacipran hydrochloride immediate release (IR) tablets.

FIG. 3 shows an expected dissolution profile for milnacipran hydrochloride delayed release (DR) tablets.

FIG. 4 shows an expected dissolution profile for milnacipran hydrochloride extended release (ER) tablets.

### DETAILED DESCRIPTION OF THE INVENTION

The present invention provides pharmaceutical formulations comprising milnacipran or a pharmaceutically acceptable salt thereof (e.g., the hydrochloride salt) and methods of treating conditions comprising administering the formulations to a patient in need thereof.

In one aspect, the present invention provides formulations comprising milnacipran or pharmaceutically acceptable salts thereof. In some embodiments, the formulations may be immediate release formulations. In other embodiments, the formulations may be extended release formulations. In still other embodiments, the formulations may be delayed release formulations.

The formulations may comprise about 10 mg to about 300 mg of milnacipran or a pharmaceutically acceptable salt thereof. In exemplary embodiments, the formulations comprise milnacipran hydrochloride. For example, the formulations may comprise about 10 mg, about 12.5 mg, about 14 mg, about 15 mg, about 20 mg, about 25 mg, about 28 mg, about 50 mg, about 56 mg, about 75 mg, about 100 mg, about 112 mg, about 125 mg, about 150 mg, about 175 mg or about 200 mg of milnacipran hydrochloride. In other embodiments, the formulations may consist essentially of milnacipran or a pharmaceutically acceptable salt thereof (e.g., milnacipran hydrochloride). In such embodiments, milnacipran or a pharmaceutically acceptable salt thereof (e.g., milnacipran hydrochloride) is the only active ingredient or therapeutic agent. Such formulations may further comprise inactive ingredients such as one or more pharmaceutically acceptable carriers, excipients or diluents. For example, the formulations may consist essentially of about 10 mg, about 12.5 mg, about 14 mg, about 15 mg, about 20 mg, about 25 mg, about 28 mg, about 50 mg, about 56 mg, about 75 mg, about 100 mg, about 112 mg, about 125 mg, about 150 mg, about 175 mg or about 200 mg milnacipran or a pharmaceutically acceptable salt thereof (e.g., milnacipran hydrochloride).

In exemplary embodiments, the formulations consist essentially of about 10 mg, about 12.5 mg, about 14 mg, about 15 mg, about 20 mg, about 25 mg, about 28 mg, about 50 mg, about 56 mg, about 75 mg, about 100 mg, about 112 mg, about 125 mg, about 150 mg, about 175 mg or about 200 mg milnacipran hydrochloride.

The pharmaceutically acceptable salts of milnacipran include, but are not limited to, salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid and maleic acid. In addition, compounds containing a carboxy group or other acidic group may be used. In some examples, the compounds may be converted into a pharmaceutically acceptable addition salt with inorganic or organic bases including, but not limited to, sodium hydroxide, potassium hydroxide, ammonia, cyclohexylamine, dicyclohexyl-amine, ethanolamine, diethanolamine and triethanolamine.

In exemplary embodiments, the formulations comprise milnacipran or a pharmaceutically acceptable salt thereof and 1-phenyl-3-aza-bicyclo[3.1.0]hexan-2-one. For example, the formulations may comprise 1-phenyl-3-aza-bicyclo[3.1.0]hexan-2-one in a concentration between about 0.01% and about 5% w/w. In some embodiments, the concentration may be between about 0.01 and about 4% w/w, about 0.01 and about 3% w/w, about 0.01 and about 2.5% w/w, about 0.1 and about 3% w/w, about 0.5 and about 3% w/w, about 0.5 and about 2.5% w/w, about 1 and about 3% w/w, about 1 and about 2.5% w/w, about 0.01 and about 2% w/w, about 0.5 and about 2% w/w or about 1 and about 2% w/w.

In exemplary embodiments, the formulations may comprise up to about 0.5% w/w 1-phenyl-3-aza-bicyclo[3.1.0]hexan-2-one. For example, the formulations may comprise 1-phenyl-3-aza-bicyclo[3.1.0]hexan-2-one in a concentration between about 0.03 and about 0.5% w/w. In specific embodiments, the concentration of 1-phenyl-3-aza-bicyclo[3.1.0]hexan-2-one may be about 0.1% w/w, about 0.2% w/w, about 0.3% w/w or about 0.4% w/w.

In some embodiments, the concentration of 1-phenyl-3-aza-bicyclo[3.1.0]hexan-2-one may be assessed within about 6 months. For example, the concentrations may be assessed within about 0 months, about 1 month, about 2 months, about 3 months, about 4 months, about 5 months, about 6 months, about 7 months, about 8 months or about 9 months. The concentration of 1-phenyl-3-aza-bicyclo[3.1.0]hexan-2-one may be assessed under different conditions. For example, the concentration may be assessed after storage of the formulations for 3 months at 40° C. and 75% relative humidity (RH). In other examples, the formulations may be stored for 1 month, 2 months, 4 months, 5 months, 6 months, 7 months, 8 months, 9 months or 12 months under different conditions. The concentration of 1-phenyl-3-aza-bicyclo[3.1.0]hexan-2-one may be used to assess the stability of a formulation comprising milnacipran. In exemplary embodiments, formulations containing less than 0.5% w/w 1-phenyl-3-aza-bicyclo[3.1.0]hexan-2-one may have improved stability.

In some embodiments, the formulations comprise 1-phenyl-3-aza-bicyclo[3.1.0]hexan-2-one in a concentration of less than about 5% w/w. In some embodiments, the formulations comprise 1-phenyl-3-aza-bicyclo[3.1.0]hexan-2-one in a concentration of less than about 4.5% w/w. In some embodiments, the formulations comprise 1-phenyl-3-aza-bicyclo[3.1.0]hexan-2-one in a concentration of less than about 4.0% w/w. In some embodiments, the formulations comprise 1-phenyl-3-aza-bicyclo[3.1.0]hexan-2-one in a concentration of less than about 3.5% w/w. In some embodiments, the formulations comprise 1-phenyl-3-aza-bicyclo[3.1.0]hexan-2-one in a concentration of less than about 3.0% w/w. In some embodiments, the formulations comprise 1-phenyl-3-aza-bicyclo[3.1.0]hexan-2-one in a concentration of less than about 2.5% w/w. In some embodiments, the formulations comprise 1-phenyl-3-aza-bicyclo[3.1.0]hexan-2-one in a concentration of less than about 2.0% w/w. In some embodiments, the formulations comprise 1-phenyl-3-aza-bicyclo[3.1.0]hexan-2-one in a concentration of less than about 1.5% w/w. In some embodiments, the formulations comprise 1-phenyl-3-aza-bicyclo[3.1.0]hexan-2-one in a concentration of less than about 1.0% w/w. In some embodiments, the formulations comprise 1-phenyl-3-aza-bicyclo[3.1.0]hexan-2-one in a concentration of less than about 0.5% w/w. In some embodiments, the formulations comprise 1-phenyl-3-aza-bicyclo[3.1.0]hexan-2-one in a concentration of less than about 0.1% w/w. In some embodiments, the composition comprises 1-phenyl-3-aza-bicyclo[3.1.0]hexan-2-one in a concentration of less than about 0.05% w/w. In some embodiments, these concentrations are assessed after storage of the composition for 3 months at 40° C. and 75% relative humidity (RH).

In some embodiments, the formulations release more than about 80% milnacipran or a pharmaceutically acceptable salt thereof within 60 minutes upon entry in a use environment. For example, the formulations may release more than about 80% milnacipran or a pharmaceutically acceptable salt thereof within about 10 minutes, about 15 minutes, about 30 minutes, about 45 minutes or about 60 minutes. In some embodiments, entry into a use environment includes, but is not limited to, contact of a formulation of the invention with the gastric or enteric fluids of a patient to whom it is administered, or with a fluid intended to simulate gastric fluid. For example, the use environment includes, but is not limited to, dissolution media (e.g., pH 6.8 buffer) commonly used for testing the dissolution rate of formulations. In some embodiments, use environment refers to the stomach or other portion of the gastrointestinal tract intended as the site of major absorption locus for the milnacipran or pharmaceutically acceptable salt thereof. The milnacipran or pharmaceutically acceptable salt thereof may be released in a dissolution medium with a pH of about 6.8. In some examples, the dissolution medium may be maintained at about 37° C.±1° C.

In exemplary embodiments, the formulations may comprise about 12.5 mg to about 200 mg milnacipran or a pharmaceutically acceptable salt thereof and about 0.03 to about 0.5% w/w 1-phenyl-3-aza-bicyclo[3.1.0]hexan-2-one and release more than about 80% milnacipran or pharmaceutically acceptable salt (e.g., milnacipran hydrochloride) thereof within 30 minutes upon entry in a use environment. In some embodiments, the formulations provide a single phase release of milnacipran or a pharmaceutically acceptable salt thereof. For example, the formulations release milnacipran or pharmaceutically acceptable salt thereof in one continuous phase (e.g., formulations that release more than 80% drug load within 30 minutes) as compared to formulations that provide a multi-phase release of milnacipran or pharmaceutically acceptable salt thereof (e.g., formulations that may be employed to attain one or more combinations of release rates to attain therapeutic objectives such as a portion of drug releasing immediately, followed by an extended release of the remainder.

In exemplary embodiments, the formulations may be immediate release tablets that provide a single phase release of more than about 80% milnacipran or a pharmaceutically acceptable salt thereof within 30 minutes after entry into a use environment. In other embodiments, the formulations may be immediate release capsules that provide a single phase release of more than about 80% milnacipran or a pharmaceutically acceptable salt thereof within 30 minutes after entry into a use environment.

IR formulations (such as IR beads) typically have a high dissolution rate such that, e.g., greater than about 80% (e.g., greater than about 85%, greater than about 90%, greater than about 95%, or even greater than about 99%) of the active contained within the IR beads is released from the formulation within about the first 60 minutes following entry of the formulation into a use environment (such as following administration to a patient). In some embodiments, the IR formulation has a dissolution rate of greater than about 80% (e.g., greater than about 85%, greater than about 90%, greater than about 95%, or even greater than about 99%) within 50 minutes, within 40 minutes, within 30 minutes, 20 minutes, or even within 10 minutes.

In some embodiments, the immediate release formulation may be a matrix. In other embodiments, the immediate release formulations may be a bead. For example, the formulations may be provided as immediate release tablets for oral administration (e.g., once daily or twice daily). In other examples, the formulations may be provided as immediate release capsules for oral administration (e.g., once daily or twice daily). The formulations may comprise milnacipran or a pharmaceutically acceptable salt thereof in a therapeutically effective amount. For example, the amount may be from about 12.5 mg to about 300 mg. In some examples, the amount may be about 12.5 mg, about 25 mg, about 50 mg, about 75 mg, about 100 mg or about 200 mg.

In exemplary embodiments, the compositions comprise about 12.5 mg, about 25 mg, about 50 mg or about 100 mg milnacipran or a pharmaceutically acceptable salt thereof (e.g., milnacipran hydrochloride) and about 0.03 to about 0.5% w/w 1-phenyl-3-aza-bicyclo[3.1.0]hexan-2-one such that the compositions provide a single phase of release of more than about 80% milnacipran or a pharmaceutically acceptable salt thereof within about 30 minutes upon entry into a use environment, for example, a dissolution medium of a pH of about 6.8.

In exemplary embodiments, the formulations may further comprise a coating. The amount of coating applied may range from about 0.5 to about 8% weight gain. In exemplary embodiments, the weight gain may be about 2 to about 6%. For example, the weight gain may be about 1%, about 2%, about 3%, about 4%, about 5%, about 6%, about 7% or about 8%.

In some examples, the coating comprises a polymer in combination with a surfactant. The polymer may be a film forming polymer, including, but not limited to, polyvinyl pyrrolidone (PVP), polyvinyl alcohol (PVA), hydroxypropyl cellulose (HPC), hydroxypropyl methylcellulose (HPMC), hydroxyethylmethyl cellulose, sodium carboxymethylcellulose, calcium carboxymethylcellulose, amylase starch, chitosan, hydroxyethyl cellulose, polydextrose, polyethylene oxide, maltodextrin and methyl cellulose. The amount of polymer may depend on the amount of milnacipran or a pharmaceutically acceptable salt thereof. For example, the mass ratio of milnacipran or a pharmaceutically acceptable salt thereof to the coating polymer may range from about 3 to about 80. In exemplary embodiments, the ratio may range from about 4 to about 70. For example, the mass ratio may be about 5, about 10, about 20, about 30, about 40, about 50, about 60 or about 70.

In some embodiments, the surfactant may be a plasticizer, such as polyethylene glycol (PEG), triacetin, glycerol, propylene glycol, acetyltributyl citrate, acetyltriethyl citrate, dibutyl phthalate, dibutyl sebacate, diethyl phthalate, glycerin, glycerin monostearate, tributyl citrate and triethyl citrate. The amount of surfactant may depend on the amount of milnacipran or a pharmaceutically acceptable salt thereof. For example, the mass ratio of milnacipran or a pharmaceutically acceptable salt thereof to the surfactant may range from about 20 to about 1000. In exemplary embodiments, the ratio may be about 40 to about 700. For example, the mass ratio may be about 50, about 100, about 150, about 200, about 250, about 300, about 350, about 400, about 450, about 500, about 550, about 600, about 650 or about 700.

In some embodiments, the ratio of the polymer to surfactant may range from about 1:1 to about 20:1. For example, the ratio may about 2:1 to about 10:1. In exemplary embodiments, the ratio may be about 2:1, about 3:1, about 4:1, about 5:1, about 6:1, about 7:1, about 8:1, about 9:1 or about 10:1. In specific embodiments, the ratio may be about 1, about 1.5, about 2, about 2.5, about 3.5, about 4, about 4.5, about 5, about 5.5, about 6, about 6.5, about 7, about 7.5, about 8, about 8.5, about 9, about 9.5, about 10, about 10.5, about 11, about 11.5 or about 12.

In exemplary embodiments, a polyvinyl alcohol (PVA) coating may be applied to formulations comprising milnacipran hydrochloride in a range of 0.5 to 8% weight gain, preferably in the range of 2 to 6%. For example, the weight gain may be about 1%, about 2%, about 3%, about 4%, about 5%, about 6%, about 7% or about 8%. In other examples, hydroxypropyl methylcellulose (HPMC) based coating may be used.

In some embodiments, the formulations may comprise a coating with an adhesion force of more than 20 N. For example, the adhesion force may be more than about 25 N, 30 N, 35 N or 40N. The adhesion force is a measure of the affinity between the film and the tablet. In some examples, the coating has an adhesion force of ≦31 N. In other examples, the coating has an adhesion force of ≧35 N. In exemplary embodiments, the coating has an adhesion force of between about 25 N and about 30 N; about 30 N and about 35 N; or about 35 N and about 40 N. In exemplary embodiments, the adhesion force may be between about 30 N and 40 N.

In exemplary embodiments, the formulations provide an in vivo plasma profile for the milnacipran or pharmaceutically acceptable salt thereof (e.g., milnacipran hydrochloride) comprising a mean Cₘₐₓ of less than about 1500 ng/ml. For example, the plasma profile comprises a mean Cₘₐₓ of less than about 100 ng/ml, less than about 200 ng/ml, less than about 300 ng/ml, less than about 400 ng/ml, less than about 500 ng/ml, less than about 600 ng/ml, less than about 700 ng/ml, less than about 800 ng/ml, less than about 900 ng/ml or less than about 1000 ng/ml. In exemplary embodiments, the plasma profile comprises a mean Cₘₐₓ of about 20 to about 1000 ng/ml. In other embodiments, the plasma profile comprises a mean Cₘₐₓ of about 20 to about 500 ng/ml.

In exemplary embodiments, the formulations comprise about 12.5 mg to 200 mg milnacipran (e.g., about 12.5 mg, about 25 mg, about 50 mg, or about 100 mg) or a pharmaceutically acceptable salt thereof (e.g., milnacipran hydrochloride) and provide an in vivo plasma profile for milnacipran or pharmaceutically acceptable salt thereof (e.g., milnacipran hydrochloride) comprising a mean Cₘₐₓ of less than about 1500 ng/ml. For example, the plasma profile comprises a mean Cₘₐₓ of less than about 100 ng/ml, less than about 200 ng/ml, less than about 300 ng/ml, less than about 400 ng/ml, less than about 500 ng/ml, less than about 600 ng/ml, less than about 700 ng/ml, less than about 800 ng/ml, less than about 900 ng/ml or less than about 1000 ng/ml. In exemplary embodiments, the plasma profile comprises a mean Cₘₐₓ of about 20 to about 1000 ng/ml. In other embodiments, the plasma profile comprises a mean Cₘₐₓ of about 200 to about 1000 ng/ml.

In exemplary embodiments, the formulations comprise about 12.5 mg to 200 mg milnacipran (e.g., about 12.5 mg, about 25 mg, about 50 mg, or about 100 mg) or a pharmaceutically acceptable salt thereof (e.g., milnacipran hydrochloride) and provide an in vivo plasma profile for the milnacipran or pharmaceutically acceptable salt thereof (e.g., milnacipran hydrochloride) comprising a mean AUC_{0-∞} of more than about 100 ng·h/ml. For example, the plasma profile comprises a mean AUG_{0-∞} of about 100 to 12000 ng·h/ml; about 100 to 10000 ng·h/ml; about 100 to 8000 ng h/ml; about 100 to 5000 ng·h/ml or about 100 to 2000 ng·h/ml. In exemplary embodiments, the plasma profile comprises a mean AUC_{0-∞} of about 200 to 5000 ng·h/ml.

In exemplary embodiments, the formulations comprise about 12.5 mg to 200 mg milnacipran (e.g., about 12.5 mg, about 25 mg, about 50 mg, or about 100 mg) or a pharmaceutically acceptable salt thereof (e.g., milnacipran hydrochloride) and provide an in vivo plasma profile for the milnacipran or pharmaceutically acceptable salt thereof (e.g., milnacipran hydrochloride) comprising a mean Tₘₐₓ of more than about 30 min. For example, the plasma profile comprises a mean Tₘₐₓ of more than about 1 hour, 1.5 hours, about 2 hours, about 3 hour or about 4 hours. In exemplary embodiments, the plasma profile comprises a mean Tₘₐₓ of about 2 hours to about 3 hours.

In another aspect, the present invention provides modified release formulations comprising milnacipran or a pharmaceutically acceptable salt thereof. Optionally, the formulations (e.g., delayed or extended release formulations) can contain one or more carriers, excipients, fillers, stabilizing agents, binders, colorants, glidants, and lubricants (all pharmaceutically acceptable). In exemplary embodiments, the present invention provides delayed release or extended release formulations comprising about 12.5 mg to about 300 mg milnacipran or a pharmaceutically acceptable salt thereof that provide a single phase release of the milnacipran or a pharmaceutically acceptable salt thereof upon entry into a use environment. Such formulations release milnacipran or pharmaceutically acceptable salt thereof in one continuous phase as compared to formulations that provide a multi-phase release of milnacipran or pharmaceutically acceptable salt thereof (e.g., formulations that may be employed to attain one or more combinations of release rates).

In certain embodiments, stable and bioavailable formulations (such as modified release formulations, delayed release formulations, or extended release formulations) comprising milnacipran and/or its salts (e.g., milnacipran hydrochloride) are described.

In some embodiments, delayed release formulations are provided that comprise from about 12.5 mg to about 300 mg milnacipran hydrochloride, wherein administration of the formulation (e.g., a single dose of the formulation) provides, produces, or achieves an in vivo plasma profile comprising (i) a mean Cₘₐₓ of less than about 960 ng/mL, (ii) a mean AUC_{0-∞} of more than about 155 ng·hr/mL and (iii) a mean Tₘₐₓ of about 3 or more hours.

In some embodiments, extended release formulations are provided that comprise from about 12.5 mg to about 300 mg milnacipran hydrochloride, wherein administration of the formulation (e.g., a single dose of the formulation) provides an in vivo plasma profile comprising (i) a mean Cₘₐₓ of less than about 540 ng/mL, (ii) a mean AUC_{0-∞} of more than about 155 ng·hr/mL and (iii) a mean Tₘₐₓ of about 3 or more hours.

In further embodiments, extended release formulations are provided that comprise from about 12.5 mg to about 300 mg milnacipran hydrochloride, wherein administration of the formulation (e.g., a single dose of the formulation) provides an in vivo plasma profile comprising (i) a mean Cₘₐₓ of less than about 625 ng/mL, (ii) a mean AUC_{0-∞} of more than about 110 ng·hr/mL and (iii) a mean Tₘₐₓ of about 4 or more hours.

In some embodiments, the formulations comprise, consist essentially of, or consist of 1-phenyl-3-aza-bicyclo[3.1.0]hexan-2-one in a concentration between about 0.01% and about 5% w/w. In some embodiments, the formulations comprise 1-phenyl-3-aza-bicyclo[3.1.0]hexan-2-one in a concentration between about 0.01 and about 4% w/w. In some embodiments, the formulations comprise 1-phenyl-3-aza-bicyclo[3.1.0]hexan-2-one in a concentration between about 0.01 and about 3% w/w. In some embodiments, the formulations comprise 1-phenyl-3-aza-bicyclo[3.1.0]hexan-2-one in a concentration between about 0.1 and about 3% w/w. In some embodiments, the formulations comprise 1-phenyl-3-aza-bicyclo[3.1.0]hexan-2-one in a concentration between about 0.5 and about 3% w/w. In some embodiments, the formulations comprise 1-phenyl-3-aza-bicyclo[3.1.0]hexan-2-one in a concentration between about 1 and about 3% w/w. In some embodiments, the formulations comprise 1-phenyl-3-aza-bicyclo[3.1.0]hexan-2-one in a concentration between about 0.01 and about 2.5% w/w. In some embodiments, the formulations comprise 1-phenyl-3-aza-bicyclo[3.1.0]hexan-2-one in a concentration between about 0.5 and about 2.5% w/w. In some embodiments, the formulations comprise 1-phenyl-3-aza-bicyclo[3.1.0]hexan-2-one in a concentration between about 1 and about 2.5% w/w. In some embodiments, the formulations comprise 1-phenyl-3-aza-bicyclo[3.1.0]hexan-2-one in a concentration between about 0.01 and about 2% w/w. In some embodiments, the formulations comprise 1-phenyl-3-aza-bicyclo[3.1.0]hexan-2-one in a concentration between about 0.5 and about 2% w/w. In some embodiments, the formulations comprise 1-phenyl-3-aza-bicyclo[3.1.0]hexan-2-one in a concentration between about 1 and about 2% w/w. In some embodiments, these concentrations are assessed after storage of the composition for 3 months at 40° C. and 75% relative humidity (RH).

In some embodiments, the composition comprises 1-phenyl-3-aza-bicyclo[3.1.0]hexan-2-one in a concentration of less than about 5% w/w. In some embodiments, the composition comprises 1-phenyl-3-aza-bicyclo[3.1.0]hexan-2-one in a concentration of less than about 4.5% w/w. In some embodiments, the composition comprises 1-phenyl-3-aza-bicyclo[3.1.0]hexan-2-one in a concentration of less than about 4.0% w/w. In some embodiments, the composition comprises 1-phenyl-3-aza-bicyclo[3.1.0]hexan-2-one in a concentration of less than about 3.5% w/w. In some embodiments, the composition comprises 1-phenyl-3-aza-bicyclo[3.1.0]hexan-2-one in a concentration of less than about 3.0% w/w. In some embodiments, the composition comprises 1-phenyl-3-aza-bicyclo[3.1.0]hexan-2-one in a concentration of less than about 2.5% w/w. In some embodiments, the composition comprises 1-phenyl-3-aza-bicyclo[3.1.0]hexan-2-one in a concentration of less than about 2.0% w/w. In some embodiments, the composition comprises 1-phenyl-3-aza-bicyclo[3.1.0]hexan-2-one in a concentration of less than about 1.5% w/w. In some embodiments, the composition comprises 1-phenyl-3-aza-bicyclo[3.1.0]hexan-2-one in a concentration of less than about 1.0% w/w. In some embodiments, the composition comprises 1-phenyl-3-aza-bicyclo[3.1.0]hexan-2-one in a concentration of less than about 0.5% w/w. In some embodiments, the composition comprises 1-phenyl-3-aza-bicyclo[3.1.0]hexan-2-one in a concentration of less than about 0.1% w/w. In some embodiments, the composition comprises 1-phenyl-3-aza-bicyclo[3.1.0]hexan-2-one in a concentration of less than about 0.05% w/w. In some embodiments, these concentrations are assessed after storage of the composition for 3 months at 40° C. and 75% relative humidity (RH).

In another aspect, the present invention provides modified release formulations, e.g., delayed release formulations or extended release formulations. In some embodiments, the modified release formulations can be prepared in a suitable manner, such as by coating an immediate release (IR) dosage form (e.g., an IR bead dosage form) with a suitable MR coating.

Milnacipran can be easily processed in solvent state with conventional binder such as Povidone. However, in aqueous state the milnacipran has some unusual properties which present difficulties in drug layering approach. Milnacipran is highly soluble in water and a deliquescent substance at 75-80% RH at room temperature. These properties make the immediate release beads of milnacipran prone to wetting and tacking during coating process. Surprisingly and unexpectedly, it has been found that the use of polyethylene glycol (PEG) enables drug layering. In exemplary embodiments, the PEG functions as a co-binder, anti-tacking agent, plasticizer to HPMC and as an eraser to the drug's deliquescent property.

It has now been found that milnacipran and its salts (e.g., milnacipran hydrochloride) can be formulated into dosage forms that have advantageous stability profiles, are bioavailable, and exhibit reduced incidence and intensity of adverse events following administration to patients as compared to conventional formulations of milnacipran. The dosage forms include immediate release tablets, immediate release beads in capsule, modified release tablets, and modified release beads in capsule.

Beads packaged in capsule offer dose flexibility, and thus are needed. Due to its deliquescent property, milnacipran can not be processed into beads in aqueous drug layering approach using conventional binders, which presents a formulation problem. Surprisingly and unexpectedly, it has now been found that excipients such as polyethylene glycol (PEG) can be used as “eraser” to reduce the milnacipran deliquescent properties, and thus enable drug layering.

In some embodiments, the IR bead and/or IR core comprises, e.g., an inert core, active drug, solvent/anti-tacking agent (such as polyethylene glycol, PEG), glidant (such as talc) and/or a binder (such as hydroxypropyl methyl cellulose, HPMC). A suitable IR bead form of milnacipran may simply be particles of milnacipran or a salt thereof (e.g., milnacipran hydrochloride) admixed with soluble components, such as, for example, sugars (e.g., sucrose, mannitol, etc.), polymers (e.g., polyethylene glycol, hydroxypropyl cellulose, hydroxypropyl methyl cellulose, etc.), surfactants (e.g., sodium lauryl sulphate, chremophor, tweens, spans, pluronics, and the like), insoluble glidant components and anti-tacking agents (e.g., PEG, microcrystalline cellulose, calcium phosphate, talc, fumed silica, and the like), coating materials (e.g., polyethylene glycol, hydroxypropyl methyl cellulose, wax, fatty acids, etc.), dispersions in suitable material (e.g., wax, polymers, pharmaceutically acceptable oils, soluble agents, etc.) or combinations or mixtures thereof.

In some embodiments, the inert core comprises, for example, sugar spheres (nonpareil seeds), microcrystalline cellulose, and/or mannitol. For example, the inert core is a sugar sphere, USP (Paulaur Cranbury, N.J.). Depending on the drug loading, the particle size of the inert core can be any suitable size, such as ranging from about 1 μm to about 1000 μm, such as from about 300 μm to about 900 μm, e.g., from about 450 μm to about 825 μm.

In some embodiments, the inert spheres comprise a portion of the drug loaded particles ranging from about 100 mg/g to about 600 mg/g, preferably from about 200 mg/g to about 500 mg/g. The fraction of the inert bead depends on any additional constituents of drug loaded beads.

In some embodiments, the inert core is coated with milnacipran or a pharmaceutically acceptable salt thereof, e.g., milnacipran hydrochloride. In one embodiment, milnacipran hydrochloride is present in an amount ranging from about 100 mg/g to about 600 mg/g, such as from about 200 to 500 mg/g based on the weight of the entire IR bead.

In certain embodiments, the milnacipran hydrochloride is added to a mixture of binder (e.g., HPMC), solvent/anti-tacking agent (e.g., PEG), and a glidant (e.g., talc) prior to coating the inert core. Solvent/anti-tacking agents may be used in amounts ranging from about 20 mg/g to about 200 mg/g, such as from about 50 mg/g to 200 mg/g or from about 100 mg/g to about 200 mg/g. Glidants may be used in amounts ranging from 1.5 mg/g to about 70 mg/g, such as from about 1.5 mg/g to about 30 mg/g or from about 8.0 mg/g to about 20 mg/g. In another embodiment, the amount of glidant used is from about 5 mg/g to about 30 mg/g.

The binder may be selected from, but not limited to, povidone (PVP), hydroxypropyl methylcellulose (HPMC, available, e.g., as Opadry®), hydroxypropyl cellulose (HPC), or combinations thereof. In an embodiment where the binder is HPMC, the binder is present in an amount ranging from about 5 mg/g to about 40 mg/g, such as from about 10 mg/g to about 30 mg/g. In another embodiment, where the binder is povidone, the binder is present in an amount of from about 1.5 mg/g to about 35 mg/g, such as from about 5 mg/g to about 30 mg/g.

Table 1 provides exemplary IR bead formulations for use in the present invention.

TABLE 1
Milnacipran HCl IR Beads

| | | Preferred | | | | | |
|---|---|---|---|---|---|---|---|
| | Range | Range | Example 1 | Example 2 | Example 3 | Example 4 | Example 5 |
| Ingredient | mg/g | mg/g | mg/g | mg/g | mg/g | mg/g | mg/g |
| Sugar Spheres, USP | 55.0-865.0 | 212.5-730.0 | 410.0 | 460.0 | 420.0 | 370.0 | 565.0 |
| Milnacipran HCl | 100-600 | 200-500 | 400.0 | 400.0 | 400.0 | 400.0 | 300.0 |
| Polyethylene Glycol, | 23.0-170.0 | 47.5-142.5 | 95.0 | 95.0 | 133.0 | 133.0 | 99.8 |
| NF (PEG 8000) | | | | | | | |
| Polyethylene Glycol, | 1.0-10.5 | 2.5-8.8 | 5.0 | 5.0 | 7.0 | 7.0 | 5.2 |
| NF (PEG 400) | | | | | | | |
| Hydroxypropyl | 6.0-36.0 | 12.0-30.0 | 24.0 | 24.0 | 24.0 | 24.0 | 18.0 |
| methylcellulose | | | | | | | |
| (Opadry ®) | | | | | | | |
| Talc, USP | 4.0-24.0 | 8.0-20.0 | 16.0 | 16.0 | 16.0 | 16.0 | 12.0 |
| Hydroxypropyl | 0-100.0 | 0-62.5 | 50.0 | — | — | 50.0 | — |
| methylcellulose | | | | | | | |
| (Opadry ®)* | | | | | | | |
| Purified Water, USP** | — | — | — | — | — | — | — |
| Total | 1000 | 1000 | 1000 | 1000 | 1000 | 1000 | 1000 |

*Optional seal coating
**Purified water is removed during the manufacturing process

The mixture of active ingredient and binder/solvent/glidant may be prepared by mixing the components, e.g., with a stirrer, for at least 15 minutes, such as for at least 30 minutes or for at least one hour. The components may also be combined by methods including blending, mixing, dissolution and evaporation, or by using suspensions.

The active ingredient/binder/inactives mixture may be deposited on inert cores, wet massed and extruded, granulated, or spray dried. In one embodiment, the sugar spheres are prewarmed to a temperature ranging from about 35° C. to about 55° C. prior to application of the mixture. The inert beads may be optionally coated with from about 2% w/w to about 10% w/w seal coating prior to applying the active drug layer. The seal coating may be any applicable coating which can separate any active ingredients from the inert core, for example, polymer coatings such as Eudragit®, HPMC, HPC and combinations thereof.

The sugar spheres may be coated using a fluidized bed coater known in the art, for example, a Glatt Powder Coater and Granulator, GPCG3 (Ramsey, N.Y.). Coating conditions such as air velocity, spray rate, and atomization pressure are typically controlled as is appreciated by and known to those skilled in the art. For example, the temperature may range from about 38° C. to about 51° C.; the air velocity may range from about 5 to about 9 m/s; the spray rate may range from about 9 to about 42 gm/min and the atomization pressure may range from about 1.5 to about 2.5 bar. The beads are then dried in the fluidized bed of the coating apparatus at a temperature of about 45° C. to about 50° C. for at least 5 minutes, preferably at least 10 minutes, more preferably at least 15 minutes. One skilled in the art will recognize that many alternate operating conditions and various types of equipment can also be used.

Once the IR beads are formed as inert cores containing coated drug, the beads may be optionally additionally coated with a seal coating. The seal coating may be a polymer or a combination of polymers. Suitable polymers include, e.g., HPMC (Opadry®, Colorcon, PA), HPC, PVP, Ethylcellulose (Aquacoat®, FMC Biopolymer, PA), Ethylcellulose (Ethocel®, Dow Chemical, USA and Germany), Eudragit® EPO (Evonik, formally Degussa, Darmstadt, Germany) and combinations of two or more of the foregoing. In the IR bead formulations, the optional seal coating polymer may be present in an amount ranging from about 2% w/w to about 8% w/w, such as about 4% w/w to about 6% w/w.

In some embodiments, the composition is a modified release (MR) formulations, e.g., delayed release or extended release formulations, such as MR bead formulations. Such modified release dosage forms can be any dosage forms whose drug-release characteristics of time course and/or location are chosen to accomplish therapeutic or convenience objectives not offered by conventional dosage forms such as a solution or an immediate release dosage form. Modified release solid oral dosage forms include both delayed and extended release drug products.

The MR formulations can be prepared in any suitable manner, such as using an initial step as described above for IR beads, with the inert core, layer of active ingredient mixture, and optional seal coating. The IR beads may then be coated with one or more MR component(s) in the form of release modifying polymer dispersion(s) to provide delayed release forms or extended release forms, and optionally an additional topcoat of polymer to increase or enhance aesthetics, handling ease, and/or stability. The final dosage form, such as a tablet or capsule, may contain a different amount of beads depending on the desired dose of the composition.

In some embodiments, the composition is a delayed release formulations, for example, any formulation that release milnacipran at a time other than immediately following oral administration. For example, enteric coated products are delayed release dosage forms intended to delay the release of a drug (or drugs) until the dosage form has passed through the stomach.

Delayed release formulations can be created in any suitable manner, such as by coating a solid dosage form with a polymer film, e.g., a polymer film that is insoluble in the acidic environment of the stomach, and soluble in the neutral environment of the small intestine. As such, the drug is not released in the stomach (pH less than about 5), but is released in the intestinal region (pH greater than about 5.5). In some embodiments, the delayed-release component of the formulation is designed to prevent drug release in the upper part of the gastrointestinal (GI) tract, thereby reducing, minimizing, managing, and/or preventing nausea, vomiting, and/or bleeding, such as due to irritation of the gastric mucosa, relative to IR formulations not comprising a delayed release component.

Suitable coating materials (or delayed release coating materials) include, for example, bioerodible, gradually hydrolyzable, gradually water-soluble, and/or enzymatically degradable polymers, such as enteric polymers. Enteric polymers become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methylcellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit™ (Rohm Pharma; Westerstadt, Germany), vinyl polymers and copolymers such as vinylpyrrolidone/vinyl acetate, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied. In one embodiment, the coating material is a methacrylic acid copolymer type C (Acryl-Eze)™.

The coating composition may also include conventional additives, such as plasticizers, pigments, colorants, stabilizing agents and glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides and the like. A stabilizing agent is typically used to stabilize particles in the dispersion. Typical stabilizing agents include emulsifiers such as sodium lauryl sulfate, sorbitan esters, polysorbates, and polyvinylpyrrolidone and the like. Glidants are typically used to reduce sticking effects during film formation and drying, and will generally represent approximately 25 wt. % to 100 wt. % of the polymer weight in the coating solution. One effective glidant is talc. Other glidants such as collidal anhydrous silica, magnesium stearate, glycerol monostearates and the like may also be used. Pigments such as titanium dioxide may also be used. Small quantities of an anti-foaming agent, such as a silicone (e.g., simethicone), may also be added to the coating composition.

In exemplary embodiments, the present invention provides delayed release oral dose bead formulations comprising a plurality of immediate release beads comprising milnacipran, or a pharmaceutically acceptable salt thereof, wherein the immediate release beads are coated with a methacryclic acid copolymer.

In some embodiments, the delayed release formulations may comprise a coating comprising a polymer such as, polyvinyl alcohol, hydroxypropylmethyl cellulose, acetylributyl citrate, carbomers, cellulose acetate phthalate, hypromellose acetate succinate, hypromellose phthalate, polymethacrylates, polyvinyl acetate phthalate, shellac, tributyl citrate, triethyl citrate and zein. In specific embodiments, the coating may be applied to the formulations (e.g., a core tablet) in a range of about 0.5 to about 8% weight gain. For example, the range may be about 2 to 6%. A delayed release (DR) coating may be applied subsequently in a range of about 5 to 90% weight gain relative to the weight of the formulation (e.g., core tablet with sub-coat). In some examples the range may be about 10% to about 80%.

Examples of polymers that can be used for preparing milnacipran delayed release (DR) tablet coating include polyvinyl alcohol, hydroxypropylmethyl cellulose, acetylributyl citrate, carbomers, cellulose acetate phthalate, hypromellose acetate succinate, hypromellose phthalate, polymethacrylates, polyvinyl acetate phthalate, shellac, tributyl citrate, triethyl citrate, zein, etc. The mass ratio of milnacipran or a pharmaceutically acceptable salt thereof to the delayed release coating polymer may be between of 3 and 100. In exemplary embodiments, the ratio may be between about 4 and 80.

The coating may further comprise a surfactant. In some embodiments, the surfactant may be a plasticizer, such as polyethylene glycol (PEG), triacetin, glycerol, propylene glycol, acetyltributyl citrate, acetyltriethyl citrate, dibutyl phthalate, dibutyl sebacate, diethyl phthalate, glycerin, glycerin monostearate, tributyl citrate and triethyl citrate. The amount of surfactant may depend on the amount of milnacipran or a pharmaceutically acceptable salt thereof. For example, the mass ratio of milnacipran or a pharmaceutically acceptable salt thereof to the surfactant may range from about 20 to about 1000. In exemplary embodiments, the ratio may be about 40 to about 700. For example, the mass ratio may be about 50, about 100, about 150, about 200, about 250, about 300, about 350, about 400, about 450, about 500, about 550, about 600, about 650 or about 700.

In some embodiments, the ratio of the polymer to surfactant may range from about 1:1 to about 20:1. For example, the ratio may about 2:1 to about 10:1. In exemplary embodiments, the ratio may be about 2:1, about 3:1, about 4:1, about 5:1, about 6:1, about 7:1, about 8:1, about 9:1 or about 10:1. In specific embodiments, the ratio may be about 1, about 1.5, about 2, about 2.5, about 3.5, about 4, about 4.5, about 5, about 5.5, about 6, about 6.5, about 7, about 7.5, about 8, about 8.5, about 9, about 9.5, about 10, about 10.5, about 11, about 11.5 or about 12.

Table 2 demonstrates exemplary embodiments of delayed release oral dose bead formulations according to the present invention.

TABLE 2
Milnacipran HCl Delayed Release Beads (DR)

| | | | Preferred | | |
|---|---|---|---|---|---|
| | Range 1 | Range 2 | Range | Example 1 | Example 2 |
| Ingredient | mg/g | mg/g | mg/g | mg/g | mg/g |
| Sugar Spheres, USP | 30.0-500.0 | 31.4-495.6 | 121.4-402.9 | 211.4 | 217.6 |
| Milnacipran HCl | 55.0-355.0 | 57.1-352.9 | 114.3-294.1 | 228.5 | 235.3 |
| Polyethylene Glycol, | 19.0-120.0 | 19.0-117.3 | 38.0-97.8 | 76.0 | 78.2 |
| NF (PEG 8000) | | | | | |
| Polyethylene Glycol, | 1.0-6.5 | 1.0-6.2 | 2.0-5.1 | 4.0 | 4.1 |
| NF (PEG 400) | | | | | |
| Hydroxypropyl | 3.0-21.0 | 3.4-21.2 | 6.9-17.6 | 13.8 | 14.1 |
| methylcellulose | | | | | |
| (Opadry ®) | | | | | |
| Talc, USP | 2.0-15.0 | 2.3-14.1 | 4.6-11.8 | 9.1 | 9.4 |
| Hydroxypropyl | 7.0-45.0 | 7.1-44.1 | 14.3-36.8 | 28.6 | 29.4 |
| methylcellulose | | | | | |
| (Opadry ®)* | | | | | |
| Methacrylic acid | 110.0-560.0 | 114.3-558.8 | 342.8-500.0 | 428.6 | 411.8 |
| copolymer type C | | | | | |
| dispersion (Acryl-EZE ®) | | | | | |
| Purified Water, USP** | | — | — | — | — |
| Total mg | 1000 | 1000 | 1000 | 1000 | 1000 |

*Optional seal coating
**Purified Water is removed during the manufacturing process.

The delayed release oral formulations can have a time of maximum plasma concentration (Tₘₐₓ) in human patients ranging from between about 3 to about 6 hours, such as, for example, between about 3.5 hours and about 5.5 hours, between about 4 to about 5 hours, or even between about 4.5 and about 5 hours.

The delay in the release of milnacipran delays or postpones the rise of milnacipran in the blood plasma to a time point that is not immediate after administration, such as, for example, greater than 1 hour, greater than 2 hours, greater than 3 hours, greater than 4 hours, between about 2 and 5 hours, between about 3 and 5 hours, or even between about 4.5 and 5 hours after oral administration, hence allowing for bed time (PM) administration.

In some embodiments, delayed release formulations are provided which comprise from about 12.5 mg to about 300 mg milnacipran, or a pharmaceutically acceptable salt thereof, wherein the single dose administration of formulation provides an in vivo plasma profile comprising (i) a mean Cₘₐₓ of less than about 960 ng/mL, (ii) a mean AUC_{0-∞} of more than about 155 ng·hr/mL and (iii) a mean Tₘₐₓ of about 3 or more hours. For example, the formulation provides an in vivo plasma profile comprising (i) a mean Cₘₐₓ of less than about 820 ng/mL, (ii) a mean AUC_{0-∞} of more than about 210 ng·hr/mL and (iii) a mean Tₘₐₓ of about 3 or more hours.

In some embodiments, delayed release formulations are provided which comprise about 12.5 mg milnacipran, or a pharmaceutically acceptable salt thereof, wherein the single dose administration of formulation provides an in vivo plasma profile comprising (i) a mean Cₘₐₓ of less than about 40 ng/mL, (ii) a mean AUG_{0-∞} of more than about 155 ng·hr/mL and (iii) a mean Tₘₐₓ of about 3 or more hours. For example, the formulation provides an in vivo plasma profile comprising (i) a mean Cₘₐₓ of less than about 35 ng/mL, (ii) a mean AUC_{0-∞} of more than about 210 ng·hr/mL and (iii) a mean Tₘₐₓ of about 3 or more hours.

In some embodiments, delayed release formulations are provided which comprise about 14 mg milnacipran, or a pharmaceutically acceptable salt thereof, wherein the single dose administration of formulation provides an in vivo plasma profile comprising (i) a mean Cₘₐₓ of less than about 45 ng/mL, (ii) a mean AUG_{0-∞} of more than about 175 ng·hr/mL and (iii) a mean Tₘₐₓ of about 3 or more hours. For example, the formulation provides an in vivo plasma profile comprising (i) a mean Cₘₐₓ of less than about 40 ng/mL, (ii) a mean AUC_{0-∞} of more than about 235 ng·hr/mL and (iii) a mean Tₘₐₓ of about 3 or more hours.

In some embodiments, delayed release formulations are provided which comprise about 15 mg milnacipran, or a pharmaceutically acceptable salt thereof, wherein the single dose administration of formulation provides an in vivo plasma profile comprising (i) a mean Cₘₐₓ of less than about 50 ng/mL, (ii) a mean AUG_{0-∞} of more than about 190 ng·hr/mL and (iii) a mean Tₘₐₓ of about 3 or more hours. For example, the formulation provides an in vivo plasma profile comprising (i) a mean Cₘₐₓ of less than about 40 ng/mL, (ii) a mean AUC_{0-∞} of more than about 250 ng·hr/mL and (iii) a mean Tₘₐₓ of about 3 or more hours.

In some embodiments, delayed release formulations are provided which comprise about 25 mg milnacipran, or a pharmaceutically acceptable salt thereof, wherein the single dose administration of formulation provides an in vivo plasma profile comprising (i) a mean Cₘₐₓ of less than about 80 ng/mL, (ii) a mean AUG_{0-∞} of more than about 315 ng·hr/mL and (iii) a mean Tₘₐₓ of about 3 or more hours. For example, the formulation provides an in vivo plasma profile comprising (i) a mean Cₘₐₓ of less than about 70 ng/mL, (ii) a mean AUC_{0-∞} of more than about 420 ng·hr/mL and (iii) a mean Tₘₐₓ of about 3 or more hours.

In some embodiments, delayed release formulations are provided which comprise about 28 mg milnacipran, or a pharmaceutically acceptable salt thereof, wherein the single dose administration of formulation provides an in vivo plasma profile comprising (i) a mean Cₘₐₓ of less than about 90 ng/mL, (ii) a mean AUG_{0-∞} of more than about 355 ng·hr/mL and (iii) a mean Tₘₐₓ of about 3 or more hours. For example, the formulation provides an in vivo plasma profile comprising (i) a mean Cₘₐₓ of less than about 75 ng/mL, (ii) a mean AUG_{0-∞} of more than about 475 ng·hr/mL and (iii) a mean Tₘₐₓ of about 3 or more hours.

In some embodiments, delayed release formulations are provided which comprise about 30 mg milnacipran, or a pharmaceutically acceptable salt thereof, wherein the single dose administration of formulation provides an in vivo plasma profile comprising (i) a mean Cₘₐₓ of less than about 95 ng/mL, (ii) a mean AUG_{0-∞} of more than about 380 ng·hr/mL and (iii) a mean Tₘₐₓ of about 3 or more hours. For example, the formulation provides an in vivo plasma profile comprising (i) a mean Cₘₐₓ of less than about 80 ng/mL, (ii) a mean AUC_{0-∞} of more than about 505 ng·hr/mL and (iii) a mean Tₘₐₓ of about 3 or more hours.

In some embodiments, delayed release formulations are provided which comprise about 45 mg milnacipran, or a pharmaceutically acceptable salt thereof, wherein the single dose administration of formulation provides an in vivo plasma profile comprising (i) a mean Cₘₐₓ of less than about 180 ng/mL, (ii) a mean AUG_{0-∞} of more than about 850 ng·hr/mL and (iii) a mean Tₘₐₓ of about 3 or more hours. For example, the formulation provides an in vivo plasma profile comprising (i) a mean Cₘₐₓ of less than about 155 ng/mL, (ii) a mean AUC_{0-∞} of more than about 1125 ng·hr/mL and (iii) a mean Tₘₐₓ of about 3 or more hours.

In some embodiments, delayed release formulations are provided which comprise about 50 mg milnacipran, or a pharmaceutically acceptable salt thereof, wherein the single dose administration of formulation provides an in vivo plasma profile comprising (i) a mean Cₘₐₓ of less than about 185 ng/mL, (ii) a mean AUG_{0-∞} of more than about 860 ng·hr/mL and (iii) a mean Tₘₐₓ of about 3 or more hours. For example, the formulation provides an in vivo plasma profile comprising (i) a mean Cₘₐₓ of less than about 155 ng/mL, (ii) a mean AUC_{0-∞} of more than about 1155 ng·hr/mL and (iii) a mean Tₘₐₓ of about 3 or more hours.

In some embodiments, delayed release formulations are provided which comprise about 56 mg milnacipran, or a pharmaceutically acceptable salt thereof, wherein the single dose administration of formulation provides an in vivo plasma profile comprising (i) a mean Cₘₐₓ of less than about 210 ng/mL, (ii) a mean AUG_{0-∞} of more than about 990 ng·hr/mL and (iii) a mean Tₘₐₓ of about 3 or more hours. For example, the formulation provides an in vivo plasma profile comprising (i) a mean Cₘₐₓ of less than about 185 ng/mL, (ii) a mean AUC_{0-∞} of more than about 1315 ng·hr/mL and (iii) a mean Tₘₐₓ of about 3 or more hours.

In some embodiments, delayed release formulations are provided which comprise about 60 mg milnacipran, or a pharmaceutically acceptable salt thereof, wherein the single dose administration of formulation provides an in vivo plasma profile comprising (i) a mean Cₘₐₓ of less than about 225 ng/mL, (ii) a mean AUG_{0-∞} of more than about 1040 ng·hr/mL and (iii) a mean Tₘₐₓ of about 3 or more hours. For example, the formulation provides an in vivo plasma profile comprising (i) a mean Cₘₐₓ of less than about 195 ng/mL, (ii) a mean AUC_{0-∞} of more than about 1385 ng·hr/mL and (iii) a mean Tₘₐₓ of about 3 or more hours.

In some embodiments, delayed release formulations are provided which comprise about 75 mg milnacipran, or a pharmaceutically acceptable salt thereof, wherein the single dose administration of formulation provides an in vivo plasma profile comprising (i) a mean Cₘₐₓ of less than about 270 ng/mL, (ii) a mean AUG_{0-∞} of more than about 1230 ng·hr/mL and (iii) a mean Tₘₐₓ of about 3 or more hours. For example, the formulation provides an in vivo plasma profile comprising (i) a mean Cₘₐₓ of less than about 235 ng/mL, (ii) a mean AUC_{0-∞} of more than about 1640 ng·hr/mL and (iii) a mean Tₘₐₓ of about 3 or more hours.

In some embodiments, delayed release formulations are provided which comprise about 100 mg milnacipran, or a pharmaceutically acceptable salt thereof, wherein the single dose administration of formulation provides an in vivo plasma profile comprising (i) a mean Cₘₐₓ of less than about 350 ng/mL, (ii) a mean AUG_{0-∞} of more than about 1525 ng·hr/mL and (iii) a mean Tₘₐₓ of about 3 or more hours. For example, the formulation provides an in vivo plasma profile comprising (i) a mean Cₘₐₓ of less than about 300 ng/mL, (ii) a mean AUG_{0-∞} of more than about 2050 ng·hr/mL and (iii) a mean Tₘₐₓ of about 3 or more hours.

In some embodiments, delayed release formulations are provided which comprise about 110 mg milnacipran, or a pharmaceutically acceptable salt thereof, wherein the single dose administration of formulation provides an in vivo plasma profile comprising (i) a mean Cₘₐₓ of less than about 380 ng/mL, (ii) a mean AUG_{0-∞} of more than about 1675 ng·hr/mL and (iii) a mean Tₘₐₓ of about 3 or more hours. For example, the formulation provides an in vivo plasma profile comprising (i) a mean Cₘₐₓ of less than about 325 ng/mL, (ii) a mean AUC_{0-∞} of more than about 2230 ng·hr/mL and (iii) a mean Tₘₐₓ of about 3 or more hours.

In some embodiments, delayed release formulations are provided which comprise about 112 mg milnacipran, or a pharmaceutically acceptable salt thereof, wherein the single dose administration of formulation provides an in vivo plasma profile comprising (i) a mean Cₘₐₓ of less than about 390 ng/mL, (ii) a mean AUG_{0-∞} of more than about 1700 ng·hr/mL and (iii) a mean Tₘₐₓ of about 3 or more hours. For example, the formulation provides an in vivo plasma profile comprising (i) a mean Cₘₐₓ of less than about 330 ng/mL, (ii) a mean AUG_{0-∞} of more than about 2270 ng·hr/mL and (iii) a mean Tₘₐₓ of about 3 or more hours.

In some embodiments, delayed release formulations are provided which comprise about 120 mg milnacipran, or a pharmaceutically acceptable salt thereof, wherein the single dose administration of formulation provides an in vivo plasma profile comprising (i) a mean Cₘₐₓ of less than about 410 ng/mL, (ii) a mean AUG_{0-∞} of more than about 1800 ng·hr/mL and (iii) a mean Tₘₐₓ of about 3 or more hours. For example, the formulation provides an in vivo plasma profile comprising (i) a mean Cₘₐₓ of less than about 350 ng/mL, (ii) a mean AUG_{0-∞} of more than about 2400 ng·hr/mL and (iii) a mean Tₘₐₓ of about 3 or more hours.

In some embodiments, delayed release formulations are provided which comprise about 150 mg milnacipran, or a pharmaceutically acceptable salt thereof, wherein the single dose administration of formulation provides an in vivo plasma profile comprising (i) a mean Cₘₐₓ of less than about 500 ng/mL, (ii) a mean AUG_{0-∞} of more than about 2175 ng·hr/mL and (iii) a mean Tₘₐₓ of about 3 or more hours. For example, the formulation provides an in vivo plasma profile comprising (i) a mean Cₘₐₓ of less than about 430 ng/mL, (ii) a mean AUC_{0-∞} of more than about 2900 ng·hr/mL and (iii) a mean Tₘₐₓ of about 3 or more hours.

In some embodiments, delayed release formulations are provided which comprise about 180 mg milnacipran, or a pharmaceutically acceptable salt thereof, wherein the single dose administration of formulation provides an in vivo plasma profile comprising (i) a mean Cₘₐₓ of less than about 600 ng/mL, (ii) a mean AUG_{0-∞} of more than about 2550 ng·hr/mL and (iii) a mean Tₘₐₓ of about 3 or more hours. For example, the formulation provides an in vivo plasma profile comprising (i) a mean Cₘₐₓ of less than about 510 ng/mL, (ii) a mean AUC_{0-∞} of more than about 3425 ng·hr/mL and (iii) a mean Tₘₐₓ of about 3 or more hours.

In some embodiments, delayed release formulations are provided which comprise about 200 mg milnacipran, or a pharmaceutically acceptable salt thereof, wherein the single dose administration of formulation provides an in vivo plasma profile comprising (i) a mean Cₘₐₓ of less than about 655 ng/mL, (ii) a mean AUG_{0-∞} of more than about 2800 ng·hr/mL and (iii) a mean Tₘₐₓ of about 3 or more hours. For example, the formulation provides an in vivo plasma profile comprising (i) a mean Cₘₐₓ of less than about 560 ng/mL, (ii) a mean AUG_{0-∞} of more than about 3750 ng·hr/mL and (iii) a mean Tₘₐₓ of about 3 or more hours.

In some embodiments, delayed release formulations are provided which comprise about 240 mg milnacipran, or a pharmaceutically acceptable salt thereof, wherein the single dose administration of formulation provides an in vivo plasma profile comprising (i) a mean Cₘₐₓ of less than about 775 ng/mL, (ii) a mean AUG_{0-∞} of more than about 3330 ng·hr/mL and (iii) a mean Tₘₐₓ of about 3 or more hours. For example, the formulation provides an in vivo plasma profile comprising (i) a mean Cₘₐₓ of less than about 665 ng/mL, (ii) a mean AUG_{0-∞} of more than about 4400 ng·hr/mL and (iii) a mean Tₘₐₓ of about 3 or more hours.

In some embodiments, delayed release formulations are provided which comprise about 300 mg milnacipran, or a pharmaceutically acceptable salt thereof, wherein the single dose administration of formulation provides an in vivo plasma profile comprising (i) a mean Cₘₐₓ of less than about 960 ng/mL, (ii) a mean AUG_{0-∞} of more than about 4075 ng·hr/mL and (iii) a mean Tₘₐₓ of about 3 or more hours. For example, the formulation provides an in vivo plasma profile comprising (i) a mean Cₘₐₓ of less than about 820 ng/mL, (ii) a mean AUC_{0-∞} of more than about 5450 ng·hr/mL and (iii) a mean Tₘₐₓ of about 3 or more hours.

In some embodiments, an extended release (ER) formulation (e.g., an extended release oral dose bead formulation) is provided that releases milnacipran over an extended period of time leading to lower peak plasma concentrations and/or to a prolonged Tₘₐₓ, as compared to an immediate release formulation of milnacipran. An extended release dosage form can be any dosage form that allows a reduction in dosing frequency as compared to that presented by a conventional dosage form, e.g., a solution or an immediate release dosage form.

In some embodiments, the ER formulation can be prepared in any suitable manner. In some embodiments, the ER formulation is prepared using an initial step of IR bead formation, such as described above, wherein the IR bead comprises an inert core, a layer of active ingredient mixture, and optionally a seal coating. The IR beads may then be coated with one or more ER component(s) in the form of release modifying polymer dispersion(s) to provide ER forms, and optionally an additional topcoat of polymer for aesthetic, handling, and/or stability purposes. The final dosage form, such as a capsule, may contain a different amount of beads depending on the desired dose of the composition.

Suitable coatings for ER formulations include, e.g., plastics, hydrophilic polymers, and fatty compounds. Suitable plastic matrices include, but are not limited to, polyvinyl chloride, polyethylene, acrylic polymers (Eudragit™), including e.g., acrylic acid and methacrylic acid copolymers, methyl methacrylate, methyl methacrylate copolymers, methyl acrylate-methyl methacrylate, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers. Suitable hydrophilic polymers include, but are not limited to, cellulosic polymers such as methyl and ethyl cellulose, hydroxyalkylcelluloses such as hydroxypropyl-cellulose, hydroxypropylmethylcellulose, hydroxypropyl methyl cellulose acetate succinate, sodium carboxymethylcellulose, polyethylene oxides and mixtures thereof. Suitable fatty compounds include, but are not limited to, various waxes such as carnauba wax and glyceryl tristearate and wax-type substances including hydrogenated castor oil or hydrogenated vegetable oil, or mixtures thereof.

In certain embodiments, the coating for the extended release formulations provides a controlled micro pH of between about 4 and about 7, e.g., about 6.5, or about 6. Any coating that maintains the micro pH between about 4 and about 7 (e.g., between about 4.5 and 6.5, between about 5 and about 6) may be used. In some embodiments, for example, the coating is ethyl cellulose having a dispersion pH of about 4-7 (e.g., Aquacoat®, FMC Biopolymer, PA).

In some embodiments, the present invention relates to an extended release pharmaceutical formulation that comprises a plurality of immediate release beads comprising milnacipran, or a pharmaceutically acceptable salt thereof, wherein the immediate release beads are coated with a an ethylcellulose dispersion (e.g., Surelease® having a pH ˜10). In some embodiments, the extended release formulations may comprise a coating. The coating may comprise a polymer, including, but not limited to, poly vinyl acetate (PVA), polyvinyl chloride, polyethylene, acrylic polymers, including e.g., acrylic acid and methacrylic acid copolymers, methyl methacrylate, methyl methacrylate copolymers, methyl acrylate-methyl methacrylate, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers, cellulosic polymers such as methyl and ethyl cellulose, hydroxyalkylcelluloses such as hydroxypropyl-cellulose, hydroxypropylmethyl cellulose, hydroxypropyl methyl cellulose acetate succinate, sodium carboxymethylcellulose, polyethylene oxides various waxes such as carnauba wax and glyceryl tristearate and wax-type substances including hydrogenated castor oil or hydrogenated vegetable oil, or mixtures thereof. The coating may be applied in the range of about 0.5 to about 8% weight gain. In exemplary embodiments, the range may be between about 2 and about 6%. An extended release coating may be subsequently applied in the range of about 1 to about 90% weight gain relative to the weight of the formulation (e.g., core tablet with sub-coat). In some examples, the range may be between about 3 and about 40%.

In exemplary embodiments, a polyvinyl alcohol (PVA) sub-coat may be applied to the formulation (e.g., a core tablet) in a range of about 0.5 to about 8% weight gain. In exemplary embodiments, the PVA coating may be applied in the range of about 2 to about 6% weight gain. The subsequent extended release coating can be applied in a range of about 1 to about 90% weight gain relative to the weight of core tablet with sub-coat, preferably in the range of about 3 to about 40%.

The coating may further comprise a surfactant. In some embodiments, the surfactant may be a plasticizer, such as polyethylene glycol (PEG), triacetin, glycerol, propylene glycol, acetyltributyl citrate, acetyltriethyl citrate, dibutyl phthalate, dibutyl sebacate, diethyl phthalate, glycerin, glycerin monostearate, tributyl citrate and triethyl citrate. The amount of surfactant may depend on the amount of milnacipran or a pharmaceutically acceptable salt thereof. For example, the mass ratio of milnacipran or a pharmaceutically acceptable salt thereof to the surfactant may range from about 20 to about 1000. In exemplary embodiments, the ratio may be about 40 to about 700. For example, the mass ratio may be about 50, about 100, about 150, about 200, about 250, about 300, about 350, about 400, about 450, about 500, about 550, about 600, about 650 or about 700.

In some embodiments, the ratio of the polymer to surfactant may range from about 1:1 to about 20:1. For example, the ratio may about 2:1 to about 10:1. In exemplary embodiments, the ratio may be about 2:1, about 3:1, about 4:1, about 5:1, about 6:1, about 7:1, about 8:1, about 9:1 or about 10:1. In specific embodiments, the ratio may be about 1, about 1.5, about 2, about 2.5, about 3.5, about 4, about 4.5, about 5, about 5.5, about 6, about 6.5, about 7, about 7.5, about 8, about 8.5, about 9, about 9.5, about 10, about 10.5, about 11, about 11.5 or about 12.

Table 3 demonstrates exemplary embodiments of an extended release oral dose bead formulation according to the present invention.

TABLE 3
Milnacipran HCl Extended Release Beads

| | | | Preferred | |
|---|---|---|---|---|
| | Range 1 | Range 2 | Range | Example 1 |
| Ingredient | mg/g | mg/g | mg/g | mg/g |
| Sugar Spheres, USP | 150.0-700.0 | 153.2-697.6 | 262.1-588.7 | 371.0 |
| Milnacipran HCl | 80.0-500.0 | 80.7-483.9 | 161.3-403.3 | 322.6 |
| Polyethylene Glycol, NF (PEG | 15.0-115.0 | 19.2-114.9 | 38.3-95.8 | 76.6 |
| 8000) | | | | |
| Polyethylene Glycol, NF (PEG | 1.0-10.0 | 1.0-6.0 | 2.0-5.0 | 4.0 |
| 400) | | | | |
| Hydroxypropyl methylcellulose | 4.0-30.0 | 4.8-29.0 | 9.7-24.2 | 19.4 |
| (Opadry ®) | | | | |
| Talc, USP | 3.0-20.0 | 3.2-19.4 | 6.5-16.1 | 12.9 |
| Ethylcellulose aqueous | 40.0-300.0 | 40.3-282.3 | 121.0-201.6 | 164.5 |
| dispersion (Surelease ®) pH | | | | |
| ~10* | | | | |
| Hydroxypropyl methylcellulose | 0-65.0 | 0-64.5 | 20.2-36.3 | 29.0 |
| (Opadry ®)** | | | | |
| Purified Water, USP*** | | — | — | — |
| Total | 1000 | 1000 | 1000 | 1000 |

*Amount indicated is as dry polymer
**Optional top coating
***Purified Water is removed during the manufacturing process.

The plasma concentration of the extended release formulation described in Table 3 have a time of maximum plasma concentration (Tₘₐₓ) in human patients ranging between about 3 and 7.5 hours, for example, between about 4 and about 6 hours, such between about 5.0 and about 5.5 hours.

In some embodiments, an extended release formulation is provided that comprises from about 12.5 mg to about 300 mg milnacipran, or a pharmaceutically acceptable salt thereof, wherein the single dose administration of formulation provides an in vivo plasma profile comprising (i) a mean Cₘₐₓ of less than about 540 ng/mL, (ii) a mean AUC_{0-∞} of more than about 155 ng·hr/mL and (iii) a mean Tₘₐₓ of about 3 or more hours. For example, the formulation provides an in vivo plasma profile comprising (i) a mean Cₘₐₓ of less than about 460 ng/mL, (ii) a mean AUC_{0-∞} of more than about 210 ng·hr/mL and (iii) a mean Tₘₐₓ of about 3 or more hours.

In some embodiments, an extended release formulation is provided that comprises about 12.5 mg milnacipran, or a pharmaceutically acceptable salt thereof, wherein the single dose administration of formulation provides an in vivo plasma profile comprising (i) a mean Cₘₐₓ of less than about 25 ng/mL, (ii) a mean AUG_{0-∞} of more than about 155 ng·hr/mL and (iii) a mean Tₘₐₓ of about 3 or more hours. For example, the formulation provides an in vivo plasma profile comprising (i) a mean Cₘₐₓ of less than about 21 ng/mL, (ii) a mean AUC_{0-∞} of more than about 210 ng·hr/mL and (iii) a mean Tₘₐₓ of about 3 or more hours.

In some embodiments, an extended release formulation is provided that comprises about 14 mg milnacipran, or a pharmaceutically acceptable salt thereof, wherein the single dose administration of formulation provides an in vivo plasma profile comprising (i) a mean Cₘₐₓ of less than about 27 ng/mL, (ii) a mean AUG_{0-∞} of more than about 175 ng·hr/mL and (iii) a mean Tₘₐₓ of about 3 or more hours. For example, the formulation provides an in vivo plasma profile comprising (i) a mean Cₘₐₓ of less than about 25 ng/mL, (ii) a mean AUC_{0-∞} of more than about 235 ng·hr/mL and (iii) a mean Tₘₐₓ of about 3 or more hours.

In some embodiments, an extended release formulation is provided that comprises about 15 mg milnacipran, or a pharmaceutically acceptable salt thereof, wherein the single dose administration of formulation provides an in vivo plasma profile comprising (i) a mean Cₘₐₓ of less than about 30 ng/mL, (ii) a mean AUG_{0-∞} of more than about 190 ng·hr/mL and (iii) a mean Tₘₐₓ of about 3 or more hours. For example, the formulation provides an in vivo plasma profile comprising (i) a mean Cₘₐₓ of less than about 25 ng/mL, (ii) a mean AUC_{0-∞} of more than about 250 ng·hr/mL and (iii) a mean Tₘₐₓ of about 3 or more hours.

In some embodiments, an extended release formulation is provided that comprises about 25 mg milnacipran, or a pharmaceutically acceptable salt thereof, wherein the single dose administration of formulation provides an in vivo plasma profile comprising (i) a mean Cₘₐₓ of less than about 65 ng/mL, (ii) a mean AUG_{0-∞} of more than about 315 ng·hr/mL and (iii) a mean Tₘₐₓ of about 3 or more hours. For example, the formulation provides an in vivo plasma profile comprising (i) a mean Cₘₐₓ of less than about 55 ng/mL, (ii) a mean AUG_{0-∞} of more than about 420 ng·hr/mL and (iii) a mean Tₘₐₓ of about 3 or more hours.

In some embodiments, an extended release formulation is provided that comprises about 28 mg milnacipran, or a pharmaceutically acceptable salt thereof, wherein the single dose administration of formulation provides an in vivo plasma profile comprising (i) a mean Cₘₐₓ of less than about 70 ng/mL, (ii) a mean AUG_{0-∞} of more than about 355 ng·hr/mL and (iii) a mean Tₘₐₓ of about 3 or more hours. For example, the formulation provides an in vivo plasma profile comprising (i) a mean Cₘₐₓ of less than about 60 ng/mL, (ii) a mean AUC_{0-∞} of more than about 470 ng·hr/mL and (iii) a mean Tₘₐₓ of about 3 or more hours.

In some embodiments, an extended release formulation is provided that comprises about 30 mg milnacipran, or a pharmaceutically acceptable salt thereof, wherein the single dose administration of formulation provides an in vivo plasma profile comprising (i) a mean Cₘₐₓ of less than about 75 ng/mL, (ii) a mean AUG_{0-∞} of more than about 380 ng·hr/mL and (iii) a mean Tₘₐₓ of about 3 or more hours. For example, the formulation provides an in vivo plasma profile comprising (i) a mean Cₘₐₓ of less than about 65 ng/mL, (ii) a mean AUC_{0-∞} of more than about 505 ng·hr/mL and (iii) a mean Tₘₐₓ of about 3 or more hours.

In some embodiments, an extended release formulation is provided that comprises about 45 mg milnacipran, or a pharmaceutically acceptable salt thereof, wherein the single dose administration of formulation provides an in vivo plasma profile comprising (i) a mean Cₘₐₓ of less than about 95 ng/mL, (ii) a mean AUG_{0-∞} of more than about 845 ng·hr/mL and (iii) a mean Tₘₐₓ of about 3 or more hours. For example, the formulation provides an in vivo plasma profile comprising (i) a mean Cₘₐₓ of less than about 85 ng/mL, (ii) a mean AUC_{0-∞} of more than about 1125 ng·hr/mL and (iii) a mean Tₘₐₓ of about 3 or more hours.

In some embodiments, an extended release formulation is provided that comprises about 50 mg milnacipran, or a pharmaceutically acceptable salt thereof, wherein the single dose administration of formulation provides an in vivo plasma profile comprising (i) a mean Cₘₐₓ of less than about 125 ng/mL, (ii) a mean AUG_{0-∞} of more than about 900 ng·hr/mL and (iii) a mean Tₘₐₓ of about 3 or more hours. For example, the formulation provides an in vivo plasma profile comprising (i) a mean Cₘₐₓ of less than about 110 ng/mL, (ii) a mean AUC_{0-∞} of more than about 1200 ng·hr/mL and (iii) a mean Tₘₐₓ of about 3 or more hours.

In some embodiments, an extended release formulation is provided that comprises about 56 mg milnacipran, or a pharmaceutically acceptable salt thereof, wherein the single dose administration of formulation provides an in vivo plasma profile comprising (i) a mean Cₘₐₓ of less than about 150 ng/mL, (ii) a mean AUG_{0-∞} of more than about 925 ng·hr/mL and (iii) a mean Tₘₐₓ of about 3 or more hours. For example, the formulation provides an in vivo plasma profile comprising (i) a mean Cₘₐₓ of less than about 125 ng/mL, (ii) a mean AUC_{0-∞} of more than about 1200 ng·hr/mL and (iii) a mean Tₘₐₓ of about 3 or more hours.

In some embodiments, an extended release formulation is provided that comprises about 60 mg milnacipran, or a pharmaceutically acceptable salt thereof, wherein the single dose administration of formulation provides an in vivo plasma profile comprising (i) a mean Cₘₐₓ of less than about 145 ng/mL, (ii) a mean AUG_{0-∞} of more than about 1030 ng·hr/mL and (iii) a mean Tₘₐₓ of about 3 or more hours. For example, the formulation provides an in vivo plasma profile comprising (i) a mean Cₘₐₓ of less than about 125 ng/mL, (ii) a mean AUC_{0-∞} of more than about 1380 ng·hr/mL and (iii) a mean Tₘₐₓ of about 3 or more hours.

In some embodiments, an extended release formulation is provided that comprises about 75 mg milnacipran, or a pharmaceutically acceptable salt thereof, wherein the single dose administration of formulation provides an in vivo plasma profile comprising (i) a mean Cₘₐₓ of less than about 170 ng/mL, (ii) a mean AUG_{0-∞} of more than about 1230 ng·hr/mL and (iii) a mean Tₘₐₓ of about 3 or more hours. For example, the formulation provides an in vivo plasma profile comprising (i) a mean Cₘₐₓ of less than about 145 ng/mL, (ii) a mean AUC_{0-∞} of more than about 1640 ng·hr/mL and (iii) a mean Tₘₐₓ of about 3 or more hours.

In some embodiments, an extended release formulation is provided that comprises about 100 mg milnacipran, or a pharmaceutically acceptable salt thereof, wherein the single dose administration of formulation provides an in vivo plasma profile comprising (i) a mean Cₘₐₓ of less than about 210 ng/mL, (ii) a mean AUG_{0-∞} of more than about 1525 ng·hr/mL and (iii) a mean Tₘₐₓ of about 3 or more hours. For example, the formulation provides an in vivo plasma profile comprising (i) a mean Cₘₐₓ of less than about 180 ng/mL, (ii) a mean AUC_{0-∞} of more than about 2050 ng·hr/mL and (iii) a mean Tₘₐₓ of about 3 or more hours.

In some embodiments, an extended release formulation is provided that comprises about 110 mg milnacipran, or a pharmaceutically acceptable salt thereof, wherein the single dose administration of formulation provides an in vivo plasma profile comprising (i) a mean Cₘₐₓ of less than about 225 ng/mL, (ii) a mean AUG_{0-∞} of more than about 1675 ng·hr/mL and (iii) a mean Tₘₐₓ of about 3 or more hours. For example, the formulation provides an in vivo plasma profile comprising (i) a mean Cₘₐₓ of less than about 195 ng/mL, (ii) a mean AUC_{0-∞} of more than about 2230 ng·hr/mL and (iii) a mean Tₘₐₓ of about 3 or more hours.

In some embodiments, an extended release formulation is provided that comprises about 112 mg milnacipran, or a pharmaceutically acceptable salt thereof, wherein the single dose administration of formulation provides an in vivo plasma profile comprising (i) a mean Cₘₐₓ of less than about 230 ng/mL, (ii) a mean AUG_{0-∞} of more than about 1700 ng·hr/mL and (iii) a mean Tₘₐₓ of about 3 or more hours. For example, the formulation provides an in vivo plasma profile comprising (i) a mean Cₘₐₓ of less than about 200 ng/mL, (ii) a mean AUC_{0-∞} of more than about 2270 ng·hr/mL and (iii) a mean Tₘₐₓ of about 3 or more hours.

In some embodiments, an extended release formulation is provided that comprises about 120 mg milnacipran, or a pharmaceutically acceptable salt thereof, wherein the single dose administration of formulation provides an in vivo plasma profile comprising (i) a mean Cₘₐₓ of less than about 245 ng/mL, (ii) a mean AUG_{0-∞} of more than about 1800 ng·hr/mL and (iii) a mean Tₘₐₓ of about 3 or more hours. For example, the formulation provides an in vivo plasma profile comprising (i) a mean Cₘₐₓ of less than about 210 ng/mL, (ii) a mean AUC_{0-∞} of more than about 2400 ng·hr/mL and (iii) a mean Tₘₐₓ of about 3 or more hours.

In some embodiments, an extended release formulation is provided that comprises about 150 mg milnacipran, or a pharmaceutically acceptable salt thereof, wherein the single dose administration of formulation provides an in vivo plasma profile comprising (i) a mean Cₘₐₓ of less than about 300 ng/mL, (ii) a mean AUG_{0-∞} of more than about 2175 ng·hr/mL and (iii) a mean Tₘₐₓ of about 3 or more hours. For example, the formulation provides an in vivo plasma profile comprising (i) a mean Cₘₐₓ of less than about 260 ng/mL, (ii) a mean AUC_{0-∞} of more than about 2900 ng·hr/mL and (iii) a mean Tₘₐₓ of about 3 or more hours.

In some embodiments, an extended release formulation is provided that comprises about 180 mg milnacipran, or a pharmaceutically acceptable salt thereof, wherein the single dose administration of formulation provides an in vivo plasma profile comprising (i) a mean Cₘₐₓ of less than about 340 ng/mL, (ii) a mean AUG_{0-∞} of more than about 2550 ng·hr/mL and (iii) a mean Tₘₐₓ of about 3 or more hours. For example, the formulation provides an in vivo plasma profile comprising (i) a mean Cₘₐₓ of less than about 300 ng/mL, (ii) a mean AUG_{0-∞} of more than about 3420 ng·hr/mL and (iii) a mean Tₘₐₓ of about 3 or more hours.

In some embodiments, an extended release formulation is provided that comprises about 200 mg milnacipran, or a pharmaceutically acceptable salt thereof, wherein the single dose administration of formulation provides an in vivo plasma profile comprising (i) a mean Cₘₐₓ of less than about 380 ng/mL, (ii) a mean AUG_{0-∞} of more than about 2800 ng·hr/mL and (iii) a mean Tₘₐₓ of about 3 or more hours. For example, the formulation provides an in vivo plasma profile comprising (i) a mean Cₘₐₓ of less than about 325 ng/mL, (ii) a mean AUC_{0-∞} of more than about 3750 ng·hr/mL and (iii) a mean Tₘₐₓ of about 3 or more hours.

In some embodiments, an extended release formulation is provided that comprises about 240 mg milnacipran, or a pharmaceutically acceptable salt thereof, wherein the single dose administration of formulation provides an in vivo plasma profile comprising (i) a mean Cₘₐₓ of less than about 450 ng/mL, (ii) a mean AUG_{0-∞} of more than about 3330 ng·hr/mL and (iii) a mean Tₘₐₓ of about 3 or more hours. For example, the formulation provides an in vivo plasma profile comprising (i) a mean Cₘₐₓ of less than about 400 ng/mL, (ii) a mean AUC_{0-∞} of more than about 4400 ng·hr/mL and (iii) a mean Tₘₐₓ of about 3 or more hours.

In some embodiments, an extended release formulation is provided that comprises about 300 mg milnacipran, or a pharmaceutically acceptable salt thereof, wherein the single dose administration of formulation provides an in vivo plasma profile comprising (i) a mean Cₘₐₓ of less than about 550 ng/mL, (ii) a mean AUG_{0-∞} of more than about 4075 ng·hr/mL and (iii) a mean Tₘₐₓ of about 3 or more hours. For example, the formulation provides an in vivo plasma profile comprising (i) a mean Cₘₐₓ of less than about 475 ng/mL, (ii) a mean AUC_{0-∞} of more than about 5450 ng·hr/mL and (iii) a mean Tₘₐₓ of about 3 or more hours.

In another exemplary embodiment, an extended release formulation is provided that comprises a plurality of immediate release beads comprising milnacipran, or a pharmaceutically acceptable salt thereof, wherein the immediate release beads are coated with an ethylcellulose dispersion (e.g., Aquacoat or Surelease™ having a pH ˜10) and a polyacrylate dispersion (e.g., Eudragit™ NE30D).

Table 4 demonstrates exemplary embodiments of additional extended release formulations according to the present invention.

TABLE 4
Milnacipran HCl Extended Release Beads

| | | | Preferred | | |
|---|---|---|---|---|---|
| | Range 1 | Range 2 | Range | Example 1 | Example 2 |
| Ingredient | mg/g | mg/g | mg/g | mg/g | mg/g |
| Sugar Spheres, USP | 115-545 | 118.8-540.6 | 203.1-456.3 | 287.5 | 268.8 |
| Milnacipran HCl | 60-380 | 62.5-375.0 | 125.0-312.5 | 250.0 | 256.0 |
| Polyethylene Glycol, | 14-90 | 14.8-89.1 | 29.7-74.2 | 59.4 | 85.1 |
| NF (PEG 8000) | | | | | |
| Polyethylene Glycol, | 0.5-5 | 0.8-4.7 | 1.6-3.9 | 3.1 | 4.5 |
| NF (PEG 400) | | | | | |
| Hydroxypropyl | 3-23 | 3.8-22.5 | 7.5-18.8 | 15.0 | 15.4 |
| methylcellulose | | | | | |
| Talc, USP | 2-20 | 2.5-15.0 | 5.0-12.5 | 10.0 | 10.2 |
| Ethylcellulose aqueous | 0-440 | 0-437.5 | 187.5-343.8 | 220.8 | 320.0 |
| dispersion¹ (e.g. Surelease ® | | | | | |
| or Aquacoat²) | | | | | |
| ^{#}Polyacrylate dispersion | 0-400 | 0-380.6 | 84.6-135.3 | 108.4 | 0 |
| (e.g., Eudragit ®, NE30D)³ | | | | | |
| Hydroxypropyl | 0-100 | 0-95.4 | 23.9-66.8 | 45.8 | 40.0 |
| methylcellulose (e.g., | | | | | |
| Opadry ®)⁴ | | | | | |
| Purified Water, USP⁵ | — | — | — | — | — |
| Total | 1000 | 1000 | 1000 | 1000 | 1000 |

¹The combined amount of ethylcellulose aquesous dispersion/polyacrylate dispersion is not zero
²Surelease is shown as an example as dry polymer
³Amount indicated is as dry polymer
⁴Optional top coating
⁵Purified water is removed during the process

In some embodiments, the extended release formulations have a time of maximum plasma concentration (Tₘₐₓ) in human patients ranging between about 4 and about 10 hours, for example, between about 5 and about 8 hours, such between about 6 and about 7.5 hours.

In some embodiments, the extended release formulations comprise from about 12.5 mg to about 300 mg milnacipran, or a pharmaceutically acceptable salt thereof, wherein the single dose administration of formulation provides an in vivo plasma profile comprising (i) a mean Cₘₐₓ of less than about 625 ng/mL, (ii) a mean AUG_{0-∞} of more than about 140 ng·hr/mL and (iii) a mean Tₘₐₓ of about 4 or more hours. For example, the formulation provides an in vivo plasma profile comprising (i) a mean Cₘₐₓ of less than about 535 ng/mL, (ii) a mean AUG_{0-∞} of more than about 180 ng·hr/mL and (iii) a mean Tₘₐₓ of about 4 or more hours.

In some embodiments, the extended release formulations comprise about 12.5 mg milnacipran, or a pharmaceutically acceptable salt thereof, wherein the single dose administration of formulation provides an in vivo plasma profile comprising (i) a mean Cₘₐₓ of less than about 20 ng/mL, (ii) a mean AUG_{0-∞} of more than about 140 ng·hr/mL and (iii) a mean Tₘₐₓ of about 4 or more hours. For example, the formulation provides an in vivo plasma profile comprising (i) a mean Cₘₐₓ of less than about 15 ng/mL, (ii) a mean AUC_{0-∞} of more than about 180 ng·hr/mL and (iii) a mean Tₘₐₓ of about 4 or more hours.

In some embodiments, the present invention relates to an extended release formulation comprising about 14 mg milnacipran, or a pharmaceutically acceptable salt thereof, wherein the single dose administration of formulation provides an in vivo plasma profile comprising (i) a mean Cₘₐₓ of less than about 20 ng/mL, (ii) a mean AUG_{0-∞} of more than about 155 ng·hr/mL and (iii) a mean Tₘₐₓ of about 4 or more hours. For example, the formulation provides an in vivo plasma profile comprising (i) a mean Cₘₐₓ of less than about 18 ng/mL, (ii) a mean AUG_{0-∞} of more than about 200 ng·hr/mL and (iii) a mean Tₘₐₓ of about 4 or more hours.

In some embodiments, the present invention relates to an extended release formulation comprising about 15 mg milnacipran, or a pharmaceutically acceptable salt thereof, wherein the single dose administration of formulation provides an in vivo plasma profile comprising (i) a mean Cₘₐₓ of less than about 25 ng/mL, (ii) a mean AUG_{0-∞} of more than about 170 ng·hr/mL and (iii) a mean Tₘₐₓ of about 4 or more hours. For example, the formulation provides an in vivo plasma profile comprising (i) a mean Cₘₐₓ of less than about 18 ng/mL, (ii) a mean AUG_{0-∞} of more than about 225 ng·hr/mL and (iii) a mean Tₘₐₓ of about 4 or more hours.

In some embodiments, the present invention relates to an extended release formulation comprising about 25 mg milnacipran, or a pharmaceutically acceptable salt thereof, wherein the single dose administration of formulation provides an in vivo plasma profile comprising (i) a mean Cₘₐₓ of less than about 40 ng/mL, (ii) a mean AUG_{0-∞} of more than about 375 ng·hr/mL and (iii) a mean Tₘₐₓ of about 4 or more hours. For example, the formulation provides an in vivo plasma profile comprising (i) a mean Cₘₐₓ of less than about 35 ng/mL, (ii) a mean AUG_{0-∞} of more than about 500 ng·hr/mL and (iii) a mean Tₘₐₓ of about 4 or more hours.

In some embodiments, the present invention relates to an extended release formulation comprising about 28 mg milnacipran, or a pharmaceutically acceptable salt thereof, wherein the single dose administration of formulation provides an in vivo plasma profile comprising (i) a mean Cₘₐₓ of less than about 40 ng/mL, (ii) a mean AUG_{0-∞} of more than about 450 ng·hr/mL and (iii) a mean Tₘₐₓ of about 4 or more hours. For example, the formulation provides an in vivo plasma profile comprising (i) a mean Cₘₐₓ of less than about 35 ng/mL, (ii) a mean AUG_{0-∞} of more than about 600 ng·hr/mL and (iii) a mean Tₘₐₓ of about 4 or more hours.

In some embodiments, the present invention relates to an extended release formulation comprising about 30 mg milnacipran, or a pharmaceutically acceptable salt thereof, wherein the single dose administration of formulation provides an in vivo plasma profile comprising (i) a mean Cₘₐₓ of less than about 40 ng/mL, (ii) a mean AUC_{0-∞} of more than about 450 ng·hr/mL and (iii) a mean Tₘₐₓ of about 4 or more hours. For example, the formulation provides an in vivo plasma profile comprising (i) a mean Cₘₐₓ of less than about 35 ng/mL, (ii) a mean AUG_{0-∞} of more than about 600 ng·hr/mL and (iii) a mean Tₘₐₓ of about 4 or more hours.

In some embodiments, the present invention relates to an extended release formulation comprising about 45 mg milnacipran, or a pharmaceutically acceptable salt thereof, wherein the single dose administration of formulation provides an in vivo plasma profile comprising (i) a mean Cₘₐₓ of less than about 80 ng/mL, (ii) a mean AUG_{0-∞} of more than about 750 ng·hr/mL and (iii) a mean Tₘₐₓ of about 4 or more hours. For example, the formulation provides an in vivo plasma profile comprising (i) a mean Cₘₐₓ of less than about 70 ng/mL, (ii) a mean AUG_{0-∞} of more than about 1000 ng·hr/mL and (iii) a mean Tₘₐₓ of about 4 or more hours.

In some embodiments, the present invention relates to an extended release formulation comprising about 50 mg milnacipran, or a pharmaceutically acceptable salt thereof, wherein the single dose administration of formulation provides an in vivo plasma profile comprising (i) a mean Cₘₐₓ of less than about 80 ng/mL, (ii) a mean AUG_{0-∞} of more than about 800 ng·hr/mL and (iii) a mean Tₘₐₓ of about 4 or more hours. For example, the formulation provides an in vivo plasma profile comprising (i) a mean Cₘₐₓ of less than about 70 ng/mL, (ii) a mean AUG_{0-∞} of more than about 1000 ng·hr/mL and (iii) a mean Tₘₐₓ of about 4 or more hours.

In some embodiments, the present invention relates to an extended release formulation comprising about 56 mg milnacipran, or a pharmaceutically acceptable salt thereof, wherein the single dose administration of formulation provides an in vivo plasma profile comprising (i) a mean Cₘₐₓ of less than about 90 ng/mL, (ii) a mean AUG_{0-∞} of more than about 875 ng·hr/mL and (iii) a mean Tₘₐₓ of about 4 or more hours. For example, the formulation provides an in vivo plasma profile comprising (i) a mean Cₘₐₓ of less than about 80 ng/mL, (ii) a mean AUG_{0-∞} of more than about 1150 ng·hr/mL and (iii) a mean Tₘₐₓ of about 4 or more hours.

In some embodiments, the present invention relates to an extended release formulation comprising about 60 mg milnacipran, or a pharmaceutically acceptable salt thereof, wherein the single dose administration of formulation provides an in vivo plasma profile comprising (i) a mean Cₘₐₓ of less than about 100 ng/mL, (ii) a mean AUG_{0-∞} of more than about 925 ng·hr/mL and (iii) a mean Tₘₐₓ of about 4 or more hours. For example, the formulation provides an in vivo plasma profile comprising (i) a mean Cₘₐₓ of less than about 90 ng/mL, (ii) a mean AUG_{0-∞} of more than about 1225 ng·hr/mL and (iii) a mean Tₘₐₓ of about 4 or more hours.

In some embodiments, the present invention relates to an extended release formulation comprising about 75 mg milnacipran, or a pharmaceutically acceptable salt thereof, wherein the single dose administration of formulation provides an in vivo plasma profile comprising (i) a mean Cₘₐₓ of less than about 115 ng/mL, (ii) a mean AUG_{0-∞} of more than about 1100 ng·hr/mL and (iii) a mean Tₘₐₓ of about 4 or more hours. For example, the formulation provides an in vivo plasma profile comprising (i) a mean Cₘₐₓ of less than about 100 ng/mL, (ii) a mean AUG_{0-∞} of more than about 1475 ng·hr/mL and (iii) a mean Tₘₐₓ of about 4 or more hours.

In some embodiments, the present invention relates to an extended release formulation comprising about 100 mg milnacipran, or a pharmaceutically acceptable salt thereof, wherein the single dose administration of formulation provides an in vivo plasma profile comprising (i) a mean Cₘₐₓ of less than about 180 ng/mL, (ii) a mean AUG_{0-∞} of more than about 1700 ng·hr/mL and (iii) a mean Tₘₐₓ of about 4 or more hours. For example, the formulation provides an in vivo plasma profile comprising (i) a mean Cₘₐₓ of less than about 155 ng/mL, (ii) a mean AUG_{0-∞} of more than about 2275 ng·hr/mL and (iii) a mean Tₘₐₓ of about 4 or more hours.

In some embodiments, the present invention relates to an extended release formulation comprising about 110 mg milnacipran, or a pharmaceutically acceptable salt thereof, wherein the single dose administration of formulation provides an in vivo plasma profile comprising (i) a mean Cₘₐₓ of less than about 160 ng/mL, (ii) a mean AUG_{0-∞} of more than about 1500 ng·hr/mL and (iii) a mean Tₘₐₓ of about 4 or more hours. For example, the formulation provides an in vivo plasma profile comprising (i) a mean Cₘₐₓ of less than about 140 ng/mL, (ii) a mean AUC_{0-∞} of more than about 2025 ng·hr/mL and (iii) a mean Tₘₐₓ of about 4 or more hours.

In some embodiments, the present invention relates to an extended release formulation comprising about 112 mg milnacipran, or a pharmaceutically acceptable salt thereof, wherein the single dose administration of formulation provides an in vivo plasma profile comprising (i) a mean Cₘₐₓ of less than about 165 ng/mL, (ii) a mean AUG_{0-∞} of more than about 1500 ng·hr/mL and (iii) a mean Tₘₐₓ of about 4 or more hours. For example, the formulation provides an in vivo plasma profile comprising (i) a mean Cₘₐₓ of less than about 140 ng/mL, (ii) a mean AUG_{0-∞} of more than about 2050 ng·hr/mL and (iii) a mean Tₘₐₓ of about 4 or more hours.

In some embodiments, the present invention relates to an extended release formulation comprising about 120 mg milnacipran, or a pharmaceutically acceptable salt thereof, wherein the single dose administration of formulation provides an in vivo plasma profile comprising (i) a mean Cₘₐₓ of less than about 175 ng/mL, (ii) a mean AUG_{0-∞} of more than about 1600 ng·hr/mL and (iii) a mean Tₘₐₓ of about 4 or more hours. For example, the formulation provides an in vivo plasma profile comprising (i) a mean Cₘₐₓ of less than about 150 ng/mL, (ii) a mean AUG_{0-∞} of more than about 2175 ng·hr/mL and (iii) a mean Tₘₐₓ of about 4 or more hours.

In some embodiments, the present invention relates to an extended release formulation comprising about 150 mg milnacipran, or a pharmaceutically acceptable salt thereof, wherein the single dose administration of formulation provides an in vivo plasma profile comprising (i) a mean Cₘₐₓ of less than about 215 ng/mL, (ii) a mean AUG_{0-∞} of more than about 1975 ng·hr/mL and (iii) a mean Tₘₐₓ of about 4 or more hours. For example, the formulation provides an in vivo plasma profile comprising (i) a mean Cₘₐₓ of less than about 185 ng/mL, (ii) a mean AUG_{0-∞} of more than about 2650 ng·hr/mL and (iii) a mean Tₘₐₓ of about 4 or more hours.

In some embodiments, the present invention relates to an extended release formulation comprising about 180 mg milnacipran, or a pharmaceutically acceptable salt thereof, wherein the single dose administration of formulation provides an in vivo plasma profile comprising (i) a mean Cₘₐₓ of less than about 250 ng/mL, (ii) a mean AUG_{0-∞} of more than about 2325 ng·hr/mL and (iii) a mean Tₘₐₓ of about 4 or more hours. For example, the formulation provides an in vivo plasma profile comprising (i) a mean Cₘₐₓ of less than about 215 ng/mL, (ii) a mean AUG_{0-∞} of more than about 3100 ng·hr/mL and (iii) a mean Tₘₐₓ of about 4 or more hours.

In some embodiments, the present invention relates to an extended release formulation comprising about 200 mg milnacipran, or a pharmaceutically acceptable salt thereof, wherein the single dose administration of formulation provides an in vivo plasma profile comprising (i) a mean Cₘₐₓ of less than about 265 ng/mL, (ii) a mean AUG_{0-∞} of more than about 2425 ng·hr/mL and (iii) a mean Tₘₐₓ of about 4 or more hours. For example, the formulation provides an in vivo plasma profile comprising (i) a mean Cₘₐₓ of less than about 225 ng/mL, (ii) a mean AUG_{0-∞} of more than about 3250 ng·hr/mL and (iii) a mean Tₘₐₓ of about 4 or more hours.

In some embodiments, the present invention relates to an extended release formulation comprising about 240 mg milnacipran, or a pharmaceutically acceptable salt thereof, wherein the single dose administration of formulation provides an in vivo plasma profile comprising (i) a mean Cₘₐₓ of less than about 330 ng/mL, (ii) a mean AUG_{0-∞} of more than about 3025 ng·hr/mL and (iii) a mean Tₘₐₓ of about 4 or more hours. For example, the formulation provides an in vivo plasma profile comprising (i) a mean Cₘₐₓ of less than about 280 ng/mL, (ii) a mean AUG_{0-∞} of more than about 4025 ng·hr/mL and (iii) a mean Tₘₐₓ of about 4 or more hours.

In some embodiments, the present invention relates to an extended release formulation comprising about 300 mg milnacipran, or a pharmaceutically acceptable salt thereof, wherein the single dose administration of formulation provides an in vivo plasma profile comprising (i) a mean Cₘₐₓ of less than about 625 ng/mL, (ii) a mean AUG_{0-∞} of more than about 4500 ng·hr/mL and (iii) a mean Tₘₐₓ of about 4 or more hours. For example, the formulation provides an in vivo plasma profile comprising (i) a mean Cₘₐₓ of less than about 535 ng/mL, (ii) a mean AUG_{0-∞} of more than about 6000 ng·hr/mL and (iii) a mean Tₘₐₓ of about 4 or more hours.

Dosage Forms

Any two or more formulations (e.g., any two or more MR formulations) described herein may be combined into a single dosage form having a uni-phase or multi-phase profile.

The formulations can comprise any desired amount or milnacipran, or pharmaceutically acceptable salt(s) thereof. In certain embodiments, the amount of the active ingredient, e.g., milnacipran hydrochloride, in the formulation is about 10 mg, about 12.5 mg, about 14 mg, about 15 mg, about 20 mg, about 25 mg, about 28 mg, about 30 mg, about 45 mg, about 50 mg, about 56 mg, about 60 mg, about 75 mg, about 100 mg, about 110 mg, about 112 mg, about 120 mg, about 150 mg, about 180 mg, about 200 mg, about 240 mg, about 250 mg or about 300 mg. In some embodiments, the formulation comprises a concentration of milnacipran that is selected from the group consisting of about 12.5 mg, about 25 mg, about 50 mg, about 100 mg or about 200 mg. In some embodiments, the formulation comprises between about 10 mg and about 300 mg, between about 12.5 mg and about 250 mg, between about 25 mg and about 200 mg, between about 50 mg and about 200 mg, between about 50 and 150 mg, or between about 100 mg and about 200 mg of milnacipran.

In some embodiments, the formulations may further comprise one or more additional carriers, excipients, fillers, stabilizing agents, binders, colorants, glidants and lubricants.

In some embodiments, the formulation comprises beads. In this regard, beads can offer advantages over conventional solid oral modified release dosage forms, such as tablets. For example, beads can be dose proportional, i.e., the same proportions of beads of different types can be used for different doses without significantly altering the percent drug released over time. For example, a 200 mg dose will deliver twice the drug as a 100 mg dose, with the same bioavailability. Different doses are obtained by using different amounts of beads. Beads also enable a variety of dissolution profiles by mixing one or more types of beads with different dissolution properties or using multi-layer coatings, as additional drug layering over a polymer layer and subsequent coatings to prepare unitary beads, as familiar to one skilled in the art. Such dissolution profiles may or may not be possible using modified release tablet formulations. Beads also enable a wide range of drug loading. One skilled in the art will recognize that higher drug loading allows for smaller capsule size.

The prolongation in the time to maximum plasma concentration (Tₘₐₓ) for the formulations described herein as compared to an immediate release formulation, is related to the release rate of the drug in the use environment. The release rate of the drug depends on many factors, including the composition of the solid dosage forms and the dissolution properties. By using different formulations containing either unitary beads or a combination of a plurality of bead types, their individual release rates can be combined to achieve desired plasma release profiles. Beads with different release characteristics can be achieved by selection of the release-modifying polymer, as well as the combination of the release-modifying polymer and the binder to impart different release characteristics to the resulting beds.

The beads or bead mixtures may be used, for example, in suspensions, filled into capsules, compressed into tablets, or filled into sachets. One or more types of extended or delayed release beads can be mixed together and encapsulated, or used as a sprinkle on the subject's food. According to the invention, the oral solid dosage form may be any of these forms. Preferably, the dosage form is a capsule.

In one embodiment of the invention, the beads are formulated into capsules with the use of an encapsulation machine. Various capsule sizes may be required to accommodate the strength and fill weight of the target formulations. Capsule size range from 00EL to 5 for fill weights ranging from about 765 mg (size 00EL) to about 10 mg (size 5).

The particle sizes of the delayed release and extended release bead components in the dosage forms depends on the technology used to prepare them. The particle sizes may range from submicron to about 500 μm for powder technologies (mixtures, spray drying, dispersions etc), from about 5 to about 1700 μm for coating technologies (Wurster®, top spray, bottom spray, spray drying, extrusion, layering, etc.), and from about 1 to about 40 μm for tabletting technologies.

Methods of Treatment

The formulations described herein can be administered for the treatment of, for example, depression, fibromyalgia syndrome (hereinafter “fibromyalgia”), chronic fatigue syndrome, pain (e.g., chronic pain, neuropathic pain such as post-herpetic neuralgia, diabetic peripheral neuropathy), attention deficit/hyperactivity disorder, visceral pain syndromes (such as irritable bowel syndrome, noncardiac chest pain, functional dyspepsia, interstitial cystitis, essential vulvodynia, urethral syndrome, orchialgia, affective disorders including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, chronic lower back pain, migraine headache, and tension headache.

In exemplary embodiments, methods are provided for treating fibromyalgia, chronic fatigue syndrome, chronic pain, neuropathic pain (e.g., post-herpetic neuralgia, diabetic peripheral neuropathy), osteoarthritis, or chronic back pain comprising administering to a patient in need thereof a formulation of the present invention.

A subject or patient in whom administration of the therapeutic compound is an effective therapeutic regimen for a disease or disorder is preferably a human, but can be any animal, including a laboratory animal in the context of a trial or screening or activity experiment. Thus, as can be readily appreciated by one of ordinary skill in the art, the methods, compounds and formulations of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., e.g., for veterinary medical use.

Milnacipran and its salts can be administered adjunctively with other active compounds such as, for example, analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

Specific examples of compounds that can be adjunctively administered with milnacipran include, but are not limited to, aceclofenac, acetaminophen, adomexetine, almotriptan, alprazolam, amantadine, amcinonide, aminocyclopropane, amitriptyline, amolodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko biloba, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In exemplary embodiments, milnacipran, or a pharmaceutically acceptable salt thereof, is administered in combination with gabapentin, pregabalin, pramipexole, 1-DOPA, amphetamine, tizanidine, clonidine, tramadol, morphine, a tricyclic antidepressant, codeine, carbamazepine, sibutramine, valium, carbamazepine or trazadone.

By adjunctive administration is meant simultaneous administration of the compounds, in the same dosage form, simultaneous administration in separate dosage forms, and separate administration of the compounds.

In some embodiments, one or more low pH polymer release modifiers (e.g., Aquacoat™, an ethylcellulose that may be obtained from FMC Biopolymer, Philadelphia, Pa., whose aqueous dispersion has a pH of 4-7) are used during the formulating process for the composition, such as to maintain a pH less than about 9. Alternatively, or in addition, organic solvents (rather than aqueous media) (e.g., ethanol, isopropanol) are used during the formulating process. In some embodiments, for example, process for preparing the formulations are provided, wherein the method comprises using an organic solvent.

### DEFINITIONS

The term “milnacipran” is used herein to refer to milnacipran, as well as any pharmaceutically acceptable salt thereof, such as milnacipran hydrochloride.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride salt, a hydrobromide salt or a mesylate salt. In one embodiment, the pharmaceutically acceptable salt is a hydrochloride salt.

The term “treating” means to relieve, alleviate, delay, reduce, reverse, improve, manage and/or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired activity upon administration to a patient in need thereof. As used herein with respect to the pharmaceutical formulations comprising milnacipran, or a pharmaceutically acceptable salt thereof, e.g., milnacipran hydrochloride, the term “therapeutically effective amount/dose” refers to the amount/dose of the compound that, when combined, is sufficient to produce an effective response upon administration to a patient.

The term “entry into a use environment” means contact of a formulation of the invention with the gastric or enteric fluids of the patient to whom it is administered, or with a fluid intended to simulate gastric fluid. As used herein, “use environment” refers to the stomach or other portion of the gastrointestinal tract intended as the site of major absorption locus for the drug.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per practice in the art. Alternatively, “about” with respect to the formulations can mean plus or minus a range of up to 20%, preferably up to 10%, more preferably up to 5%.

The pharmacokinetic parameters described herein include area under the plasma concentration-time curve (AUC₀₋₁ and AUC_{0-∞}), maximum plasma concentration (Cₘₐₓ), time of maximum plasma concentration (Tₘₐₓ) and terminal elimination half-life (T_{1/2}). The time of maximum concentration, Tₘₐₓ, is determined as the time corresponding to Cₘₐₓ Area under the plasma concentration-time curve up to the time corresponding to the last measurable concentration (AUC₀₋₁) is calculated by numerical integration using the linear trapezoidal rule as follows:

$${AUC}_{0 - t} = {\sum }_{i = 2}^{n} 0.5 \cdot ( {C}_{i} + {C}_{i - 1} ) \cdot ( {t}_{i} - {t}_{i - 1} ) \qquad Eq . 1$$

where Cᵢ is the plasma milnacipran concentrations at the corresponding sampling time point t, and n is the number of time points up to and including the last quantifiable concentration.

The terminal half-life (T_{1/2}) is calculated using the following equation:

$${T}_{1 / 2} = \frac{0.693}{{λ}_{z}} \qquad Eq . 2$$

where λ_{z} is the terminal elimination rate constant.

The area under the plasma concentration-time curve from time zero to infinity is calculated according to the following equation:

$${AUC}_{0 - ∞} = {AUC}_{0 - t} + \frac{{C}_{last}}{{λ}_{z}} \qquad Eq . 3$$

where Cₗₐₛₜ is the last measurable concentration.

The terms “consists essentially of”, “consisting essentially of”, and variants thereof, when used to refer to the composition, are used herein to mean that the composition includes milnacipran and other desired additives and pharmaceutically inactive components, but no other active pharmaceutical ingredient(s) or therapeutic agents.

All weight percentages (i.e., “% by weight” and “wt. %” and w/w) referenced herein, unless otherwise indicated, are measured relative to the total weight of the pharmaceutical composition.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

The term “F1612” as used in the examples refers to 1-phenyl-3-aza-bicyclo[3.1.0]hexan-2-one.

The amount of milnacipran is determined using reverse-phase HPLC with UV detection at 220 nm.

The amount of degradation product F1612 is determined using reverse-phase HPLC with UV detection at 220 nm.

Dissolution rates were measured using various USP Apparatus. Apparatus II (paddle) with 900 ml of 0.1N HCl was used for testing immediate release tablets/capsules. Apparatus I (basket) with 900 ml of pH 6.8 buffer was used for testing extended release beads/capsules. Apparatus I (basket) was used for testing the delayed release beads/capsules, with 900 ml of 0.1N HCl in the acid stage and 900 ml of pH 6.8 buffer in the buffer stage.

### Example 1

### Immediate Release (IR) Capsule Formulations Containing Milnacipran Hydrochloride

Immediate release capsule formulations comprising milnacipran hydrochloride were prepared according to Tables 5 and 6.

TABLE 5
Milnacipran HCl Master Blend (238 mg/g)

| Ingredient | mg/capsule | |
|---|---|---|
| Milnacipran HCl | 50.0 | |
| Dicalcium phosphate dihydrate, USP | 101.8 | |
| Calcium carboxymethyl cellulose, NF | 44.32 | |
| Povidone, USP | 4.2 | |
| Colloidal silica dioxide, NF | 1.28 | |
| Talc, USP | 4.2 | |
| Magnesium stearate, NF | 4.2 | |
| Purified Water, USP* | — | |
| Ethyl alcohol anhydrous* | — | |
| TOTAL | 210 | mg |

*Purified Water and Ethyl alcohol anhydrous is removed during the manufacturing process.

TABLE 6
Milnacipran HCl IR Capsules, 50 mg

| | Theoretical Weight | |
|---|---|---|
| Ingredient | (mg/capsule) | |
| Weight of Gelatin Capsules | 48 | mg |
| | | (Size 3) |
| Weight of Milnacipran HCl Master | 210 | mg |
| Blend, 238 mg/g | | |
| Total Gross Weight | 258 | mg/capsule |

Purified water, anhydrous ethyl alcohol and Povidone (K30) were added to a stainless steel mixing tank and mixed until the Povidone K30 had been dissolved to produce the granulating solution. Milnacipran hydrochloride, dicalcium phosphate dihydrate and calcium carboxymethyl cellulose were screened using an oscillating bar mill. The screened material was added to the high shear wet granulator and premixed for three minutes. The granulation solution was then added under an atmosphere of nitrogen. Following liquid addition, wet massing was conducted under the nitrogen blanket. Nitrogen was removed from the granulator and the granulation was wet milled using an oscillating bar mill. The milled material was added to a fluid bed and dried. The dried material was again passed through an oscillating bar mill.

The extra-granulation components were screened using an oscillating bar mill. The milnacipran hydrochloride granulation, calcium carboxymethyl cellulose, Aerosil 200 VV and talc were added to a v-blender and mixed. Magnesium sterate was then added and mixing was continued to produce the milnacipran hydrochloride master blend described in Table 5.

Using an automatic encapsulation machine, the desired amount of milnacipran hydrochloride master blend was dosed into gelatin capsules (size 3) to produce the milnacipran hydrochloride capsules shown in Table 6, which were then polished.

The dissolution rate for the IR capsules is shown in Table 7.

TABLE 7
IR Capsule Dissolution Rate

| | % Dissolved |
|---|---|
| Time (mins) | IR Capsule |
| 10 | 58 |
| 15 | 99 |
| 30 | 104 |
| 45 | 104 |

### Example 2

### IR Tablet Formulations Containing Milnacipran Hydrochloride

Immediate release (IR) tablet formulations comprising milnacipran hydrochloride were prepared. Polyvinyl pyrrolidone (PVP) was dissolved in purified water with stirring until a clear solution was obtained. Milnacipran HCl, dicalcium phosphate powder and calcium carboxymethyl cellulose were added to the bowl of a high shear granulator and mixed till uniform. The PVP solution was then added to the granulator under mixing and chopping until fine and uniform granules were formed. The granules were dried in a fluid bed followed by milling with a Quadro Comil to a desired granule particle size. The granules were then blended with calcium carboxymethyl cellulose, colloidal silica dioxide and talc. The blend was compressed dose-proportionally into tablets of four strengths: 12.5, 25, 50 and 100 mg. The IR tablets obtained were then coated with Hydroxypropyl methylcellulose (HPMC) based coating system or Polyvinyl alcohol (PVA) based coating system in a coating pan with weight gain of 3%. Table 8 provides the compositions of HPMC-coated 12.5 mg, 25 mg, 50 mg and 100 mg tablets.

TABLE 8
HPMC-coated Milnacipran HCl IR Tablets, 12.5 mg, 25 mg,
50 mg and 100 mg

| | Tablet Potency | | | |
|---|---|---|---|---|
| Ingredient | 12.5 mg | 25 mg | 50 mg | 100 mg |
| Milnacipran HCl | 12.50 | 25.00 | 50.00 | 100.00 |
| Dicalcium phosphate dihydrate, | 5.78 | 11.56 | 23.12 | 46.24 |
| USP (milled, Calipharm ® D) | | | | |
| Dicalcium phosphate dihydrate, | 22.30 | 44.60 | 89.20 | 178.40 |
| USP (unmilled, DiTab ®) | | | | |
| Calcium carboxymethyl cellulose, | 11.62 | 23.24 | 46.48 | 92.96 |
| NF | | | | |
| Povidone, USP | 1.10 | 2.20 | 4.40 | 8.80 |
| Colloidal silica dioxide, NF, EP | 0.32 | 0.64 | 1.28 | 2.56 |
| Talc, USP | 1.10 | 2.20 | 4.40 | 8.80 |
| Magnesium stearate, NF, EP, BP | 0.28 | 0.56 | 1.12 | 2.24 |
| Core Tablet Weight | 55.0 | 110.0 | 220.0 | 440.0 |
| HPMC-based film coating system | 1.65 | 3.30 | 6.60 | 13.20 |
| (Opadry ®) | | | | |
| TOTAL | 56.65 | 113.30 | 226.60 | 453.20 |

Table 9 provides the dissolution data for the HPMC-coated tablets in 0.1N HCl dissolution medium maintained at 37±0.5° C.

TABLE 9
Dissolution data for HPMC-coated milnacipran HCl IR Tablets

% Dissolved

| Time (mins) | 12.5 mg | 25 mg | 50 mg | 100 mg |
|---|---|---|---|---|
| 15 | 96 | 97 | 97 | 94 |
| 30 | 96 | 101 | 97 | 99 |
| 45 | 96 | 101 | 97 | 100 |

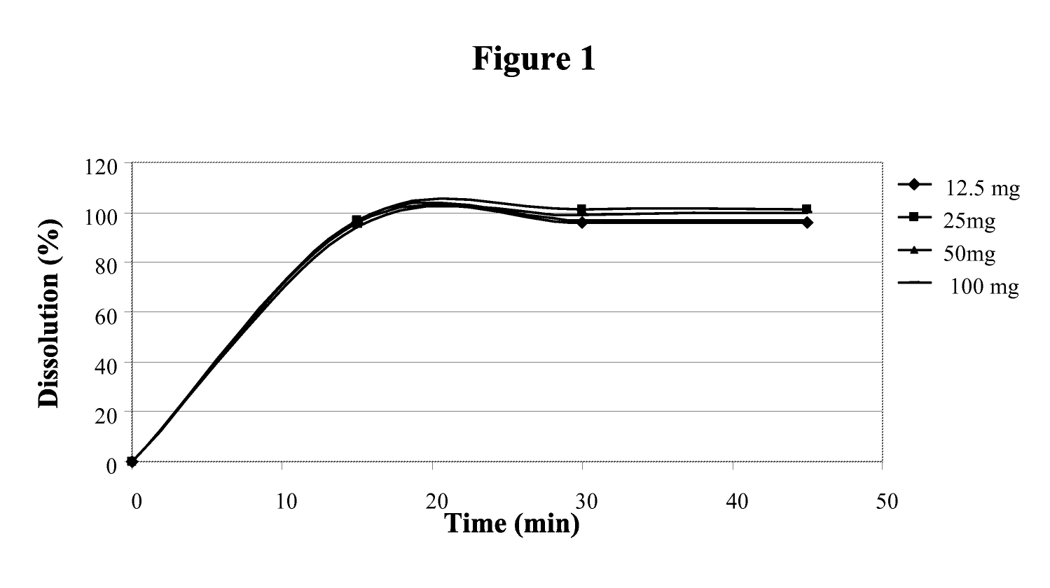

FIG. 1 shows the dissolution profile for HPMC-coated milnacipran hydrochloride immediate release (IR) tablets.

The concentration of milnacipran (illustrated in “assay” column) as well as the degradation product F1612 in the 100 mg IR tablet formulation were tested over a 6 month period (at 40° C./75% RH), as illustrated in Table 10. Table 10 shows the stability data for tablets in bottle package (with desiccant and induction sealed) and for tablets in blister packs.

TABLE 10
100 mg IR Tablet Formulation

| | 60 count (100 cc bottles) | | Blister Packs | |
|---|---|---|---|---|
| | Amount of F1612 | | Amount of F1612 | |
| Time | (% w/w) | Assay % | (% w/w) | Assay % |
| Initial | <0.05 | 97.4 | <0.05 | 97.4 |
| 1 Month | <0.05 | 97.8 | <0.05 | 97.2 |
| 3 Months | 0.08 | 95.8 | 0.15 | 95.0 |
| 6 Months | 0.12 | 95.1 | 0.42 | 93.1 |

Table 11 shows the stability data for HPMC-coated milnacipran HCl IR tablets in a bi-laminate blister.

TABLE 11
HPMC-coated Milnacipran HCl IR tablets in blister at 40° C./75% RH

| | | Amount of |
|---|---|---|
| Strength | Time | F1612 (% w/w) |
| 12.5 mg | initial | <0.05 |
| | 3 months | 0.23 |
| 25 mg | initial | <0.05 |
| | 3 months | 0.22 |
| 50 mg | initial | <0.05 |
| | 3 months | 0.16 |
| 100 mg | initial | <0.05 |
| | 3 months | 0.15 |

Table 12 shows the compositions of PVA coated 12.5 mg, 25 mg, 50 mg and 100 mg tablets.

TABLE 12
PVA-coated Milnacipran HCl IR tablets

| | Tablet Potency | | | |
|---|---|---|---|---|
| Ingredient | 12.5 mg | 25 mg | 50 mg | 100 mg |
| Milnacipran HCl | 12.50 | 25.00 | 50.00 | 100.00 |
| Dicalcium phosphate dihydrate, | 5.78 | 11.56 | 23.12 | 46.24 |
| USP (milled, Calipharm ® D) | | | | |
| Dicalcium phosphate dihydrate, | 22.30 | 44.60 | 89.20 | 178.40 |
| USP (unmilled, DiTab ®) | | | | |
| Calcium carboxymethyl cellulose, | 11.62 | 23.24 | 46.48 | 92.96 |
| NF | | | | |
| Povidone, USP | 1.10 | 2.20 | 4.40 | 8.80 |
| Colloidal silica dioxide, NF, EP | 0.32 | 0.64 | 1.28 | 2.56 |
| Talc, USP | 1.10 | 2.20 | 4.40 | 8.80 |
| Magnesium stearate, NF, EP, BP | 0.28 | 0.56 | 1.12 | 2.24 |
| Core Tablet Weight | 55.0 | 110.0 | 220.0 | 440.0 |
| PVA-based film coating system | 1.65 | 3.30 | 6.60 | 13.20 |
| (Opadry ® II) | | | | |
| TOTAL | 56.65 | 113.30 | 226.60 | 453.20 |

Table 13 provides the dissolution data for PVA-coated milnacipran HCl IR Tablets in pH 6.8 dissolution medium maintained at 37±0.5° C.

TABLE 13
Dissolution of PVA-coated milnacipran HCl IR Tablets

% Dissolved

| Time (mins) | 12.5 mg | 25 mg | 50 mg | 100 mg |
|---|---|---|---|---|
| 15 | 103 | 101 | 92 | 89 |
| 30 | 103 | 102 | 97 | 96 |
| 45 | 103 | 102 | 96 | 98 |

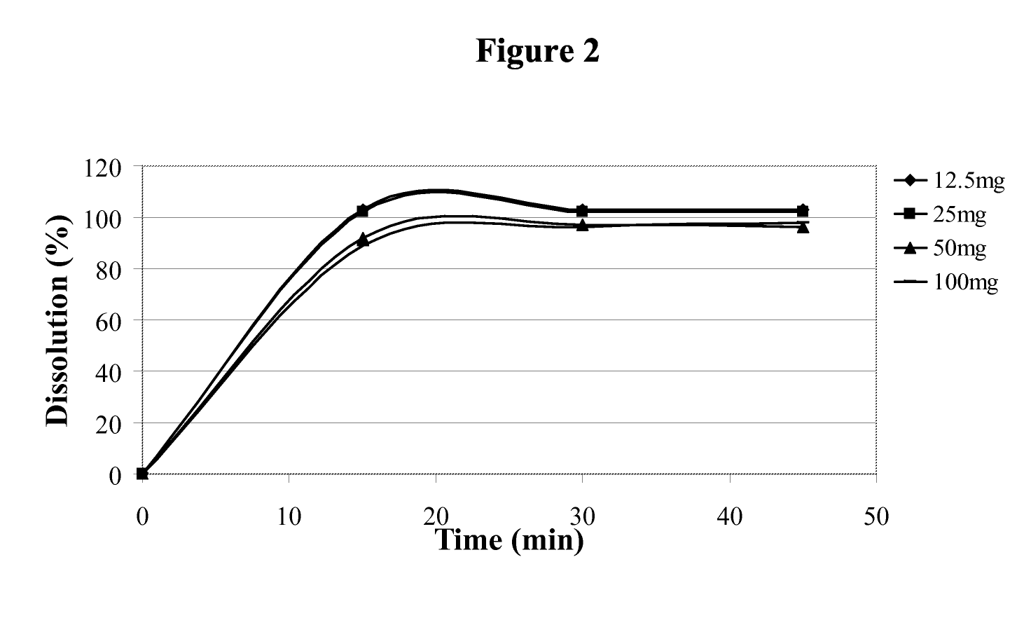

FIG. 2 shows the dissolution profile for PVA-coated milnacipran hydrochloride immediate release (IR) tablets.

Logo-bridging was observed during the tablet coating process for HPMC coated tablets, which was more pronounced in small tablets (12.5 mg and 25 mg) with weight gain of up to 3%. The adhesion of the film to the core tablet (the tendency of the film to cling to the tablet surface) was determined as an indicator for the affinity between the film and the tablet. To compare the adhesion of HPMC and PVA film to the core tablet, flat face compacts were prepared by compressing the milnacipran blend using an automatic single station hydraulic press fitted with 10 mm tooling. A theoretical weight gain of 3% film coat (HPMC or PVA) was applied to the flat face compact under standard coating conditions. Adhesion of the applied film to the compacts was determined using an Instron 5542 material tester.

TABLE 14
Film Adhesion Measurement for Milnacipran HCl IR Tablets, 25 mg

Results (N = 10)

| Film | Maximum load (N) | Work to break (mJ) |
|---|---|---|
| HPMC film | 31.41 | 0.588 |
| PVA 18442 film | >36.59 | >0.642 |

Surprisingly and unexpectedly, the maximum adhesion force of PVA film to milnacipran core tablet was found to be >36.59 N, relative to 31.4 N for HPMC film.

Surprisingly and unexpectedly, it was found that bridging of film is related to poor film adhesion to the substrate and PVA based coating system was found to demonstrate superior adhesion qualities compared to the HPMC formulation. Logo bridging was not observed at coating of 2.5% weight gain but became evident at a weight gain of 3%. Studies demonstrate that no-logo bridging occurred during the PVA coating of Milnacipran HCl tablet at up to 5% weight gain. Furthermore, it was found that other quality attributes of a tablet were not changed when PVA coating was used compared to HPMC coating.

Table 15 shows the stability data for PVA-coated Milnacipran HCl IR tablets in bi-laminate blister which is comparable to the stability data for HPMC-coated tablets as shown in Table 11.

TABLE 15
PVA-coated Milnacipran HCl IR Tablets 40° C./75% RH

| | | Amount of |
|---|---|---|
| Strength | Time | F1612 (% w/w) |
| 12.5 mg | initial | <0.05 |
| | 3 months | 0.23 |
| 25 mg | initial | <0.05 |
| | 3 months | ND |
| 50 mg | initial | <0.05 |
| | 3 months | 0.18 |
| 100 mg | initial | 0.06 |
| | 3 months | 0.22 |

ND: Not Determined

### Example 3

### Delayed Release Bead Formulations Containing Milnacipran Hydrochloride

Delayed release bead formulations containing milnacipran hydrochloride were prepared according to Tables 16-21.

TABLE 16
Milnacipran HCl IR Beads, 400 mg/g

| | Theoretical | Theoretical |
|---|---|---|
| | Weight | Weight |
| Ingredient | mg/g | mg/g |
| Sugar Spheres, USP (30-35 Mesh) | 410.0 | 370.0 |
| Milnacipran HCl | 400.0 | 400.0 |
| Polyethylene Glycol, NF (PEG 8000) | 95.0 | 133.0 |
| Polyethylene Glycol, NF (PEG 400) | 5.0 | 7.0 |
| HPMC-based Film Coating System | 24.0 | 24.0 |
| (Opadry ®) | | |
| Talc, USP | 16.0 | 16.0 |
| HPMC-based Film Coating System | 50.0 | 50.0 |
| (Opadry ®)* | | |
| Purified Water, USP** | — | — |
| Total | 1000 | 1000 |

*Optional seal coating
**Purified Water is removed during the manufacturing process

TABLE 17
Milnacipran HCl Delayed Release Beads (DR)

| | DR Bead Strength | | | |
|---|---|---|---|---|
| | | | | 224 |
| | 228 mg/g | 217 mg/g | 235 mg/g | mg/g |
| Milnacipran HCl IR Beads, | 571.5 | 542.9 | 588.2 | 558.8 |
| 400 mg/g | | | | |
| Methacrylic acid copolymer | 428.5 | 407.1 | 411.8 | 391.2 |
| (Acryl-EZE ®) | | | | |
| Purified Water, USP* | — | — | — | — |
| PVA-based coating system | — | 50.0 | — | 50.0 |
| (Opadry ® II)* | | | | |
| Purified Water, USP* | — | — | — | — |
| TOTAL | 1000 | 1000 | 1000 | 1000 |

*Purified Water is removed during the manufacturing process.

TABLE 18
Milnacipran HCl Delayed Release Capsules (DR),
12.5 mg, 25 mg, 50 mg and 100 mg

| | DR Capsule Strength | | | | | | | |
|---|---|---|---|---|---|---|---|---|
| | 12.5 mg | | 25 mg | | 50 mg | | 100 mg | |
| Weight of Gelatin Capsule | 38 | mg | 48 | mg | 76 | mg | 96 | mg |
| Shell | | (size 4) | | (size 3) | | (size 1) | | (size 0) |
| Weight of Milnacipran HCl DR | 55 | mg | 110 | mg | 219 | mg | 439 | mg |
| Beads, 228 mg/g | | | | | | | | |
| Total Gross Weight | 93 | mg | 158 | mg | 295 | mg | 535 | mg |
| | | mg/capsule | | mg/capsule | | mg/capsule | | mg/capsule |

TABLE 19
Milnacipran HCl Delayed Release Capsules (DR)

| | DR Capsule Strength | | | | | | | |
|---|---|---|---|---|---|---|---|---|
| | 12.5 mg | | 25 mg | | 50 mg | | 100 mg | |
| Weight of Gelatin Capsule | 38 | mg | 48 | mg | 76 | mg | 96 | mg |
| Shell | | (size 4) | | (size 3) | | (size 1) | | (size 0) |
| Weight of Milnacipran | 58 | mg | 115 | mg | 230 | mg | 461 | mg |
| HCl DR Beads, 217 mg/g | | | | | | | | |
| Total Gross Weight | 96 | mg/ | 163 | mg/ | 306 | mg/ | 557 | mg/ |
| | | capsule | | capsule | | capsule | | capsule |

TABLE 20
Milnacipran HCl Delayed Release Capsules (DR), 50 mg and 100 mg

| | DR Capsule Strength | | | |
|---|---|---|---|---|
| | 50 mg | | 100 mg | |
| Weight of HPMC Capsule Shell | 75 | mg | 95 | mg |
| | (size 1) | | (size 0) | |
| Weight of Milnacipran HCl DR Beads, | 213 | mg | 426 | mg |
| 235 mg/g | | | | |
| Total Gross Weight | 288 | mg/ | 521 | mg/ |
| | | capsule | | capsule |

TABLE 21
Milnacipran HCl Delayed Release Capsules (DR), 50 mg and 100 mg

| | DR Capsule Strength | | | |
|---|---|---|---|---|
| | 50 mg | | 100 mg | |
| Weight of HPMC Capsule Shell | 75 | mg | 95 | mg |
| | | (size 1) | | (size 0) |
| Weight of Milnacipran HCl DR Beads, | 223 | mg | 446 | mg |
| 224 mg/g | | | | |
| Total Gross Weight | 298 | mg/ | 541 | mg/ |
| | | capsule | | capsule |

Preparation of Milnacipran HCl IR Beads, 400 mg/g (Composition in Table 16)

Sugar spheres, USP (30-35 mesh) were coated in a fluid bed with a pre-prepared drug layering solution of milnacipran hydrochloride, polyethylene glycol, NF (PEG 8000), polyethylene glycol, NF (PEG 400), hydroxypropyl methylcellulose (Opadry®) and talc, USP dispersed in purified water, USP. After coating with the drug layering solution, a seal coating of hydroxypropyl methylcellulose (Opadry®) dispersion was applied. The beads were dried and then discharged from the fluid bed. The discharged beads were then sieved to produce the milnacipran hydrochloride IR Beads, 400 mg/g shown in Table 16.

### Preparation of Milnacipran HCl DR Beads, (Composition in Table 17)

The milnacipran hydrochloride IR beads described in Table 16 were coated with a methacrylic acid copolymer type C dispersion (Acryl-EZE®) in a fluid bed and then dried. Such Methacrylic acid copolymer coated beads may be continued to coat with a layer of PVA based film coating system (Opadry® II 85F white) and then dried. After being discharged from the fluid bed, the beads were sieved to obtain the final delayed release bead product shown in Table 17.

### Preparation of Milnacipran HCl DR Capsules, 50 mg and 100 mg (Composition in Tables 18-21)

Using an encapsulation machine, size 0, size 1, size 3 or size 4 capsules were filled with the milnacipran hydrochloride DR Beads described in Table 17 to the appropriate fill weight to afford the milnacipran HCl DR Capsules, 12.5 mg, 25 mg, 50 mg or 100 mg shown in Table 18-21.

The dissolution rate for the delayed release capsules of Table 18 is shown in Table 22.

TABLE 22
Delayed Release Capsule Dissolution Rate

| Dissolution | | |
|---|---|---|
| Stage | Time (mins) | % Dissolved |
| Acidic Stage | 60 | 0 |
| pH 1.2 | 120 | 1 |
| Buffer Stage | 135 | 100 |
| pH 6.8 | 150 | 105 |
| | 165 | 105 |

The concentration of milnacipran (illustrated in “assay” column) as well as the concentration of F1612 in the delayed release beads formulations was assayed over a 6 month period (at 40° C./75% RH), and is illustrated in Table 23.

TABLE 23
Delayed Release Beads Formulation at 40° C./75% RH

| | Amount of F1612 | % |
|---|---|---|
| Time | (% w/w) | Assay |
| Initial | <0.05 | 104.3 |
| 1 Month | <0.05 | 106.4 |
| 2 Months | <0.05 | 102.5 |
| 3 Months | 0.06 | 105.7 |
| 6 Months | 0.09 | 105.0 |

The concentration of milnacipran (illustrated in “assay” column) as well as the concentration of F1612 in the delayed release beads formulations was assayed over a 6 month period (at 30° C./65% RH), and is illustrated in Table 24.

TABLE 24
Delayed Release Beads Formulation at 30° C./65% RH

| | Amount of F1612 | % |
|---|---|---|
| Time | (% w/w) | Assay |
| Initial | <0.05 | 104.3 |
| 3 Month | <0.05 | 106.2 |
| 6 Months | <0.05 | 104.6 |
| 9 Months | 0.05 | 104.4 |
| 12 months | 0.05 | ND |

ND: Not Determined

The concentration of milnacipran (illustrated in “assay” column) as well as the concentration of F1612 in the delayed release beads formulations was assayed over a 6 month period (at 25° C./60% RH), and is illustrated in Table 25.

TABLE 25
Delayed Release Beads Formulation at 25° C./60% RH

| | Amount of F1612 | % |
|---|---|---|
| Time | (% w/w) | Assay |
| Initial | <0.05 | 104.3 |
| 6 Months | <0.05 | 104.8 |

The stability data for delayed release capsules (in gelatin capsule shell, composition in Table 18) under different packaging configurations is shown in Tables 26-28. Bottle configuration A is 100 capsules in a 120 cc HDPE bottle, induction sealed, without desiccant. Bottle configuration B is 30 capsules in a 60 cc HDPE bottle, induction sealed, without desiccant.

TABLE 26
Delayed Release Capsules at 40° C./75% RH

| | Bottle configuration A | | Bottle configuration B | |
|---|---|---|---|---|
| | Amount of F1612 | | Amount of F1612 | |
| Time | (% w/w) | % Assay | (% w/w) | % Assay |
| Initial | <0.05 | 100.0 | <0.05 | 100.0 |
| 1 Month | 0.06 | 101.5 | 0.06 | 102.5 |
| 2 Months | 0.08 | 103.3 | 0.08 | 102.5 |
| 3 Months | 0.11 | 102.1 | 0.13 | 102.1 |
| 6 Months | 0.38 | 101.2 | 0.50 | ND |

ND: Not Determined

TABLE 27
Delayed Release Capsules at 30° C./65% RH

| | Bottle configuration A | | Bottle configuration B | |
|---|---|---|---|---|
| | Amount of F1612 | | Amount of F1612 | |
| Time | (% w/w) | % Assay | (% w/w) | % Assay |
| Initial | <0.05 | 100.0 | <0.05 | 100.0 |
| 3 Months | <0.05 | 102.7 | <0.05 | 102.6 |
| 6 Months | <0.05 | 103.3 | ND | ND |

ND: Not Determined

TABLE 28
Delayed Release Capsules at 25° C./60% RH

Bottle configuration A

| | Amount of F1612 | % |
|---|---|---|
| Time | (% w/w) | Assay |
| Initial | <0.05 | 100.0 |
| 6 Months | <0.05 | 103.2 |
| 24 Months | <0.05 | 104.3 |

### Example 4

### A Single-Center, Randomized, Open-Label, Single-Dose, Parallel-Group Study for Delayed-Release Capsules, Compared to IR Capsules

A single-center, randomized, open-label, single-dose, parallel-group study in 30 male and 30 female healthy subjects (ages 18-45 years, ˜1:1 ratio male:female) was conducted to evaluate the oral bioavailability and tolerability of a delayed release formulation of milnacipran hydrochloride relative to an immediate release formulation of milnacipran hydrochloride.

The subjects were randomized with a 1:1 ratio to receive one of two treatments:

Treatment A: Single dose of 1×50 mg milnacipran hydrochloride IR capsule (see Table 6)

Treatment B: Single dose of 1×50 mg milnacipran hydrochloride DR capsule (see Table 18).

The study procedure was as follows:

Day −14 to Day −2: Potential subjects underwent screening evaluation;

Day −2: Subjects were institutionalized in a non-smoking environment;

Day −1: Subjects remained institutionalized and underwent a 10 hour fast prior to dosing on Day 1:

Day 1: Subjects received study medication at 0800 with 240 mL water. Blood samples were collected at 0 (pre-dose), 1, 2, 3, 4, 5, 6, 7, 8, 10, 12 and 14 hours post dose;

Day 2: Blood samples were collected at 24 and 36 hours post dose;

Day 3: Blood samples were collected at 48 and 60 hours post dose;

Day 4: A blood sample was collected at 72 hours post dose.

The total duration of each subjects participation was 6 days (Day −2 through the last pharmacokinetic sample collection on Day 4).

The mean pharmacokinetic parameters after administration of the single dose of 50 mg IR formulation (Treatment A) and the 50 mg DR formulation (Treatment B) are shown below in Table 29.

TABLE 29
Mean PK Parameters and Statistical Comparisons
(Subjects Without Vomiting)

| | Treatment A | Treatment B | | |
|---|---|---|---|---|
| | 50 mg IR | 50 mg DR | DR/IR | |
| | Formulation | Formulation | Ratio | |
| Parameter | (n = 22) | (n = 25) | (%) | 90% CI |
| Cₘₐₓ (ng/mL) | 134.3 ± 27.5 | 129.8 ± 31.7 | 95.5 | 85.8-107.3 |
| AUC₀₋ₜ (ng · h/mL) | 1234 ± 256 | 1360 ± 263 | 110.6 | 99.1-123.6 |
| AUC_{0-∞} (ng · h/mL) | 1345 ± 264 | 1449 ± 256 | 108.2 | 97.6-120.0 |
| T_{1/2} (h) | 7.7 ± 1.9 | 8.1 ± 1.5 | 1.04 | — |
| Tₘₐₓ (h) | 2.9 ± 1.3 | 4.7 ± 1.1 | 1.6 | — |

The incidence of adverse events (nausea, vomiting) observed during this study is shown in Table 30.

TABLE 30
Adverse Events

| | Treatment A | Treatment B |
|---|---|---|
| | (N = 30) | (N = 30) |
| Adverse Event | n (%) | n (%) |
| Nausea | 13 (43.3) | 8 (26.7) |
| Vomiting | 8 (26.7) | 5 (16.7) |

As can be seen from Table 30, nausea and vomiting rates were substantially reduced (by 38% and 39%, respectively) for the delayed release formulation relative to the immediate release formulation.

### Example 5

### Extended Release (ER) Bead Formulations Containing Milnacipran Hydrochloride

An extended release bead formulation containing milnacipran hydrochloride was prepared according to Tables 31 and 34-35.

TABLE 31
Milnacipran HCl IR Beads, 400 mg/g

| | Theoretical |
|---|---|
| | Weight |
| Ingredient | mg/g |
| Sugar Spheres, USP (30-35 Mesh) | 460.0 |
| Milnacipran HCl | 400.0 |
| Polyethylene Glycol, NF (PEG 8000) | 95.0 |
| Polyethylene Glycol, NF (PEG 400) | 5.0 |
| Hydroxypropyl methylcellulose | 24.0 |
| (Opadry ®) | |
| Talc, USP | 16.0 |
| Purified Water, USP* | — |
| Total | 1000.0 |

*Purified Water is removed during the manufacturing process

Table 32 provides the dissolution data for IR bead formulation (Table 31). USP Apparatus I (basket) was used, with 900 mL of pH 6.8 buffer dissolution medium.

TABLE 32
IR Beads Dissolution Rate

| | % Dissolved |
|---|---|
| Time (mins) | IR Beads |
| 15 | 96 |
| 30 | 98 |
| 45 | 98 |

Table 33 provides the stability data for the IR bead formulation (Table 31) at 40° C./75% RH. IR beads are packed in 60 cc HDPE bottles without desiccant, and with induction seal.

TABLE 33
IR beads Formulation 400 mg/g at 40° C./75% RH

| | Amount of F1612 | |
|---|---|---|
| Time | (% w/w) | Assay |
| Initial | <0.05 | 100.3 |
| 1 Month | <0.05 | 101.3 |
| 3 Months | <0.05 | 99.4 |
| 6 Months | 0.06 | 100.7 |

TABLE 34
Milnacipran HCl Extended Release Beads (ER1), 323 mg/g

| | Theoretical | |
|---|---|---|
| Ingredient | Weight, mg/g | |
| Milnacipran HCl IR Beads, 400 mg/g | 806.5 | |
| Ethylcellulose aqueous dispersion | 164.5 | |
| (Surelease ®) pH ~10* | | |
| HPMC-based film coating system | 29.0 | |
| (Opadry ®) | | |
| Purified Water, USP** | — | |
| TOTAL | 1000 | mg |

*Amount indicated is as a dry polymer
**Purified Water is removed during the manufacturing process.

TABLE 35
Milnacipran HCl Extended Release Capsules (ER1), 60 mg

| | Theoretical Weight |
|---|---|
| Ingredient | (mg/capsule) |
| Weight of Gelatin Capsule Shell | 76 mg (Size 1) |
| Weight of Milnacipran HCl ER Beads, | 186 mg |
| 323 mg/g | |
| Total Gross Weight | 262 mg/capsule |

Preparation of Milnacipran HCl IR Beads, 400 mg/g (Composition in Table 31)

Sugar spheres, USP (30-35 mesh) were coated in a fluid bed with a pre-prepared drug layering dispersion of milnacipran hydrochloride, polyethylene glycol, NF (PEG 8000), polyethylene glycol, NF (PEG 400), hydroxypropyl methylcellulose (Opadry®) and talc, USP in purified water, USP. The beads were dried and then discharged from the fluid bed. The discharged beads were then sieved to produce the milnacipran hydrochloride IR Beads, 400 mg/g shown in Table 31.

Preparation of Milnacipran HCl ER beads, 323 mg/g (Composition in Table 34)

The milnacipran hydrochloride IR beads described in Table 31 were coated with an ethylcellulose aquesous dispersion (Surelease®) in a fluid bed and then dried. The beads were then coated with an aquesous solution of hydroxypropyl methylcellulose (Opadry®) in the fluid bed and then dried. After being discharged from the fluid bed, the thus formed extended release beads were sieved to obtain the final extended release bead product shown in Table 34.

### Preparation of Milnacipran HCl ER Capsules, 60 mg (Composition in Table 35, ER1)

Using an encapsulation machine, size 1 capsules were filled with the milnacipran hydrochloride ER Beads described in Table 34 to the appropriate fill weight to afford the milnacipran HCl ER capsules, 60 mg shown in Table 35.

The dissolution rate for the extended release capsules described in Table 35 (ER) is shown in Table 36.

TABLE 36
Dissolution Rate for Extended Release Capsules

| | % Dissolved |
|---|---|
| Time (hr) | ER Capsules, 60 mg |
| 1 | 36 |
| 2 | 58 |
| 4 | 85 |
| 6 | 95 |
| 8 | 99 |
| 10 | 101 |
| 12 | 102 |

The concentration of milnacipran (illustrated in “assay” column) as well as the degradation product F1612 in the extended release beads described in Table 34 were tested over a 6 month period (at 40° C./75% RH), and is illustrated in Table 37.

TABLE 37
ER Beads 323 mg/g Formulation at 40° C./75% RH

| | Amount of F1612 | % |
|---|---|---|
| Time | (% w/w) | Assay |
| Initial | <0.05 | 98.0 |
| 1 Month | 0.10 | 99.4 |
| 2 Months | 0.16 | 98.3 |
| 3 Months | 0.21 | 98.0 |
| 6 Months | 0.31 | 98.9 |

The concentration of milnacipran (illustrated in “assay” column) as well as the concentration of F1612 in the extended release beads described in Table 34 was assayed over a 9 month period (at 30° C./55% RH), and is illustrated in Table 38.

TABLE 38
ER Beads at 30° C./65% RH

| | Amount of F1612 | % |
|---|---|---|
| Time | (% w/w) | Assay |
| Initial | <0.05 | 98.0 |
| 3 Month | 0.07 | 98.8 |
| 6 Months | 0.10 | 99.5 |
| 9 Months | 0.12 | ND |

ND: Not Determined

Stability data of the extended release capsules described in Table 35 under different packaging conditions on is shown in Tables 39-41. Bottle configurations A is 100 capsules in a 120 cc HDPE bottle, induction sealed, without desiccant. Bottle configurations B is 30 capsules in a 45 cc HDPE bottle, induction sealed, without desiccant.

TABLE 39
60 mg ER Capsules at 40° C./75% RH

| | Bottle configuration A | | Bottle configuration B | |
|---|---|---|---|---|
| | Amount of F1612 | % | Amount of F1612 | % |
| Time | (% w/w) | Assay | (% w/w) | Assay |
| Initial | <0.05 | 100.8 | <0.05 | 100.8 |
| 1 Month | 0.16 | 100.0 | 0.16 | 99.0 |
| 2 Months | 0.43 | 97.3 | 0.44 | 97.5 |
| 3 Months | 0.47 | 97.9 | 0.47 | 97.2 |

TABLE 40
60 mg ER Capsules at 30° C./65% RH

| | Bottle configuration A | | Bottle configuration B | |
|---|---|---|---|---|
| | Amount of F1612 | % | Amount of F1612 | % |
| Time | (% w/w) | Assay | (% w/w) | Assay |
| Initial | <0.05 | 100.8 | <0.05 | 100.8 |
| 3 Months | 0.11 | 98.2 | 0.11 | 98.7 |

ND: Not Determined

TABLE 41
60 mg ER Capsules at 25° C./60% RH

| | Bottle configuration A | | Bottle configuration B | |
|---|---|---|---|---|
| | Amount of F1612 | % | Amount of F1612 | % |
| Time | (% w/w) | Assay | (% w/w) | Assay |
| Initial | <0.05 | 100.8 | <0.05 | 100.8 |
| 3 Months | 0.07 | 101.0 | 0.09 | 101.0 |
| 6 Months | 0.08 | 98.9 | 0.05 | 98.9 |

### Example 6

### Extended Release Bead Formulation Containing Milnacipran Hydrochloride

An extended release bead formulation containing milnacipran hydrochloride was prepared according to Tables 42-44.

TABLE 42
Milnacipran HCl IR Beads, 400 mg/g

| | Theoretical |
|---|---|
| | Weight |
| Ingredient | mg/g |
| Sugar Spheres, USP (30-35 Mesh) | 460.0 |
| Milnacipran HCl | 400.0 |
| Polyethylene Glycol, NF (PEG 8000) | 95.0 |
| Polyethylene Glycol, NF (PEG 400) | 5.0 |
| Hydroxypropyl methylcellulose | 24.0 |
| (Opadry ®) | |
| Talc, USP | 16.0 |
| Purified Water, USP* | — |
| Total | 1000.0 |

*Purified Water is removed during the manufacturing process

TABLE 43
Milnacipran HCl Extended Release Beads, 250 mg/g (ER2)

| | Theoretical |
|---|---|
| | Weight |
| Ingredients | mg/g |
| Milnacipran HCl Beads, 400 mg/g | 625.0 |
| Ethylcellulose aqueous dispersion (e.g. Surelease ®, or | 220.8 |
| Aquacoat¹) | |
| Polyacrylate dispersion (Eudragit ®, NE30D)² | 108.4 |
| HPMC-based film coating system (Opadry ®) | 45.8 |
| Purified Water, USP³ | — |
| TOTAL | 1000 |

¹Surelease is shown as quantity of dry polymer,
²Amount indicated is as dry polymer, which is mixture of NE30D and Talc.
³Purified Water is removed during manufacturing process

TABLE 44
Milnacipran HCl ER Capsules, 60 mg (ER2)

| | Theoretical Weight | |
|---|---|---|
| Ingredient | (mg/capsule) | |
| Weight of Gelatin Capsule Shell | 76 | mg (size 1) |
| Weight of Milnacipran HCl ER Beads, | 240 | mg |
| 250 mg/g | | |
| Total | 316 | mg/capsule |

Preparation of Milnacipran HCl IR Beads, 400 mg/g (Composition in Table 42)

Sugar spheres, USP (30-35 mesh) were coated in a fluid bed with a pre-prepared drug layering dispersion of milnacipran hydrochloride, polyethylene glycol, NF (PEG 8000), polyethylene glycol, NF (PEG 400), hydroxypropyl methylcellulose (Opadry®) and talc, USP in purified water, USP. The beads were dried and then discharged from the fluid bed. The discharged beads were then sieved to produce the milnacipran hydrochloride IR Beads, 400 mg/g shown in Table 42.

Preparation of Milnacipran HCl ER beads, 250 mg/g (Composition in Table 43)

The milnacipran hydrochloride IR beads described in Table 42 were coated with an ethylcellulose aqueous dispersion (Surelease®) in a fluid bed and then dried. The beads were then coated with an aqueous polyacrylate dispersion (Eudragit® NE 30D) followed by a layer of HPMC-based coating system (Opadry®) aqueous solution in the fluid bed and then dried. After being discharged from the fluid bed, the thus formed extended release beads were sieved to obtain the final extended release bead product shown in Table 43.

### Preparation of Milnacipran HCl ER Capsules, 60 mg (Composition in Table 44)

Using an encapsulation machine, size 1 capsules were filled with the milnacipran hydrochloride ER beads 250 mg/g described in Table 43 to the appropriate fill weight to afford the milnacipran HCl ER capsules, 60 mg shown in Table 44.

The dissolution rate for the ER capsules of Table 44 is shown in Table 45.

TABLE 45
Dissolution Rate for Extended Release (ER2) Capsules, 60 mg

| Time (hr) | % Dissolved |
|---|---|
| 1 | 12 |
| 2 | 32 |
| 4 | 55 |
| 6 | 68 |
| 8 | 79 |
| 10 | 87 |
| 12 | 92 |

The concentration of milnacipran (illustrated in “assay” column) as well as the degradation product F1612 in the extended release beads of Table 43 were tested over a 6 month period (at 40° C./75% RH), and is illustrated in Table 46.

TABLE 46
Extended Release (ER2) Bead formulation, 250 mg/g,
at 40° C./75% RH

| | Amount of F1612 | % |
|---|---|---|
| Time | (% w/w) | Assay |
| Initial | <0.05 | 101.0 |
| 1 Month | 0.17 | 99.8 |
| 2 Months | 0.28 | 97.8 |
| 3 Months | 0.37 | 95.8 |
| 6 Months | 0.60 | 95.9 |

The concentration of milnacipran (illustrated in “assay” column) as well as the degradation product F1612 in the extended release beads of Table 43 was assayed over a 9 month period (at 30° C./65% RH), and is illustrated in Table 47.

TABLE 47
ER2 Beads at 30° C./65% RH

| | Amount of F1612 | % |
|---|---|---|
| Time | (% w/w) | Assay |
| Initial | <0.05 | 101.0 |
| 3 Month | 0.10 | 97.3 |
| 6 Months | 0.16 | 98.0 |
| 9 Months | 0.22 | 98.3 |

Stability data of the extended release capsules (ER2) described in Table 44 under different packaging configurations is shown in Tables 48-50. The packaging configuration A is 100 capsules in a 120 cc HDPE bottle, induction sealed, without desiccant. Packaging configuration B is 30 capsules in a 45 cc HDPE bottle, induction sealed, without desiccant.

TABLE 48
60 mg ER Capsules at 40° C./75% RH

| | Bottle configuration A | | Bottle configuration B | |
|---|---|---|---|---|
| | Amount of F1612 | % | Amount of F1612 | % |
| Time | (% w/w) | Assay | (% w/w) | Assay |
| Initial | <0.05 | 98.1 | <0.05 | 98.1 |
| 1 Month | 0.24 | 96.0 | 0.23 | 95.9 |
| 2 Months | 0.48 | 96.2 | 0.48 | 96.7 |
| 3 Months | 0.61 | 95.6 | 0.62 | 96.6 |
| 6 Months | 1.24 | 93.3 | 1.26 | 94.1 |

TABLE 49
60 mg ER Capsules at 30° C./65% RH

Bottle configuration B

| | Amount of F1612 | % |
|---|---|---|
| Time | (% w/w) | Assay |
| Initial | <0.05 | 98.1 |
| 3 Months | 0.14 | 96.3 |
| 6 Months | 0.25 | 97.0 |
| 9 Months | 0.37 | ND |

ND: Not Determined

TABLE 50
60 mg ER Capsules at 25° C./60% RH

Bottle configuration B

| | Amount of F1612 | % |
|---|---|---|
| Time | (% w/w) | Assay |
| Initial | <0.05 | 98.1 |
| 3 Months | 0.07 | 95.5 |
| 6 Months | 0.13 | 97.7 |
| 9 Months | 0.17 | ND |

ND: Not Determined

### Example 7

### A Single-Center, Randomized, Open-Label, Single-Dose, 3-Way Crossover Study for Extended-Release Capsules, Compared to IR Capsules

A single-center, randomized, open-label, single-dose, 3-way crossover study with a 7-day washout period in 12 male and 12 female healthy subjects (ages 18-45 years) was conducted to evaluate the oral bioavailability and tolerability of extended release formulations of milnacipran relative to an immediate release formulation of milnacipran (IR).

The subjects were randomized to receive the following three treatments separated by a 7-day washout period:

Treatment A: Single dose of 1×50 mg milnacipran hydrochloride IR capsule (Composition in Table 6 in Example 1)

Treatment B: Single dose of 1×60 mg milnacipran hydrochloride extended release capsule (Composition in Table 35 in Example 5) (“ER1”).

Treatment C: Single dose of 1×60 mg milnacipran hydrochloride extended release capsule (Composition in Table 44 in Example 6) (“ER2”).

The study procedure was as follows:

Day −14 to Day −2: Potential subjects underwent screening evaluation;

Day −2: Subjects were institutionalized in a non-smoking environment;

Day −1: Subjects remained institutionalized until ˜48 hours after the Day 1 dose, and underwent a 10 hour fast prior to dosing on Day 1:

Day 1: Subjects were randomized to receive Treatment A, B or C. The subjects received the study medication at 0800 with 240 mL water. Blood samples were collected at 0.0 (pre-dose), 0.5, 1, 2, 3, 4, 5, 6, 7, 8, 10, 12 and 14 hours post Day 1 dose;

Day 2: Blood samples were collected at 24 and 36 hours post Day 1 dose;

Day 3: A blood sample was collected at 48 hours post Day 1 dose then the patient was discharged;

Days 4-6: The washout period continued on an out-patient basis;

Day 7: Subjects were institutionalized in a non-smoking environment underwent a 10 hour fast prior to dosing on Day 8:

Day 8: Subjects received the study medication at 0800 with 240 mL water. Blood samples were collected at 0.0 (pre-dose), 0.5, 1, 2, 3, 4, 5, 6, 7, 8, 10, 12 and 14 hours post Day 8 dose;

Day 9: Blood samples were collected at 24 and 36 hours post Day 8 dose;

Day 10: A blood sample was collected at 48 hours post Day 8 dose then the patient was discharged;

Days 11-13: The washout period continued on an out-patient basis;

Day 14: Subjects were institutionalized in a non-smoking environment underwent a 10 hour fast prior to dosing on Day 15:

Day 15: Subjects received the study medication at 0800 with 240 mL water. Blood samples were collected at 0.0 (pre-dose), 0.5, 1, 2, 3, 4, 5, 6, 7, 8, 10, 12 and 14 hours post Day 8 dose;

Day 16: Blood samples were collected at 24 and 36 hours post Day 15 dose;

Day 17: A blood sample was collected at 48 hours post Day 15 dose then the patient was discharged.

The total duration of each subjects participation was 19 days (Day −2 through the last pharmacokinetic sample collection on Day 17).

The mean pharmacokinetic parameters after administration of a single dose of 50 mg IR capsule (“Treatment A”), a single dose of 60 mg extended release capsules produced according to Table 35 “ER1” (“Treatment B”) and a single dose of 60 mg extended release capsules produced according to Table 44 “ER2” (“Treatment C”) are shown in Table 51.

TABLE 51
Mean PK Parameters and Statistical Comparisons (Subjects Without Vomiting)

| | Treatment A | Treatment B | Treatment C | B vs A | C vs A |
|---|---|---|---|---|---|
| | (50 mg IR) | (60 mg ER1) | (60 mg ER2) | Ratio % | Ratio % |
| Parameter | (n = 16) | (n = 16) | (n = 16) | (90% CI) | (90% CI) |
| Cₘₐₓ | 118.8 ± 24.9 | 100.1 ± 16.7 | 66.9 ± 11.8 | 87.2 | 61.8 |
| (ng/mL) | | | | (80.3-94.6) | (56.9-67.1) |
| AUC₀₋ₜ | 1379 ± 337 | 1610 ± 292 | 1391 ± 282 | 123.3 | 108.4 |
| (ng · h/mL) | | | | (116.0-131.0) | (102.0-115.3) |
| AUC_{0-∞} | 1493 ± 342 | 1728 ± 330 | 1574 ± 343 | 121.2 | 111.2 |
| (ng · h/mL) | | | | (114.8-128.0) | (105.7-118.0) |
| T_{1/2} (h) | 9.9 ± 1.8 | 11.0 ± 2.2 | 13.0 ± 3.4 | — | — |
| Tₘₐₓ (h) | 2.1 ± 1.2 | 5.1 ± 0.7 | 6.6 ± 1.3 | — | — |

As can be seen from Table 51, the Treatment B extended release bead formulation (60 mg dose) has approximately 21% greater bioavailability as compared to the 50 mg IR formulation, and a reduced (˜16%) Cₘₐₓ relative to the IR formulation. The Treatment C extended release formulation has a similar bioavailability (AUC_{0-∞}) when compared to the IR formulation, but has greatly reduced (˜50%) peak plasma concentration (Cₘₐₓ) relative to the IR formulation.

The incidence of adverse events (nausea, vomiting) observed during this study is shown in Table 52.

TABLE 52
Observed Adverse Events

| | Treatment A | | |
|---|---|---|---|
| | IR | Treatment B | Treatment C |
| | Formulation | ER1 Formulation | ER2 Formulation |
| | (N = 23) | (N = 22) | (N = 24) |
| Adverse Event | N (%) | N (%) | N (%) |
| Nausea | 12 (52.2) | 3 (13.6) | 4 (16.7) |
| Vomiting | 7 (30.4) | 2 (9.1) | 1 (4.2) |

The nausea and vomiting incidence by treatment and period is shown in Table 53.

TABLE 53
Nausea and Vomiting Incidence by Treatment and Period

| | | | |
|---|---|---|---|
| | Treatment A | Treatment B | Treatment C |
| Period | (IR) | (ER1) | (ER2) |

Nausea

| | | | |
|---|---|---|---|
| 1 | 5 | 2 | 2 |
| 2 | 3 | 1 | 2 |
| 3 | 4 | 0 | 0 |

Vomiting

| | | | |
|---|---|---|---|
| 1 | 3 | 1 | 1 |
| 2 | 2 | 1 | 0 |
| 3 | 2 | 0 | 0 |

As can be seen from Tables 52 and 53, both the extended release formulations

(Treatments B and C) provided substantially improved GI tolerability (e.g., reduced incidence nausea and vomiting) when compared to the immediate release formulation (Treatment A).

### Example 8

### Linear Calculated Pharmacokinetic Parameters

### IR Formulation

The pharmacokinetics of the IR milnacipran hydrochloride formulation (prepared as described in Example 1, Table 6) over the single-dose range of 25 mg to 300 mg suggest approximate dose proportionality of exposure relative to mean AUC. Linear calculated pharmacokinetic parameters for the IR formulation are shown in Table 54.

TABLE 54
Linear Calculated Pharmacokinetic Parameters for IR Formulation

| | Mean Cₘₐₓ | Mean AUC_{0-∞} | Mean Tₘₐₓ |
|---|---|---|---|
| Dose (mg) | (ng/mL) | (ng/mL * h) | (hrs) |
| 12.5 | 27.2 | 265.4 | 2.2 |
| 14 | 30.5 | 297.3 | 2.2 |
| 15 | 32.7 | 318.5 | 2.2 |
| 25 | 60.7 | 697.6 | 2.2 |
| 28 | 61.0 | 876.0 | 2.2 |
| 30 | 65.3 | 837.1 | 2.2 |
| 45 | 127.3 | 1416.5 | 2.2 |
| 50 | 129.2 | 1492.1 | 3.0 |
| 56 | 151.2 | 1650.1 | 3.0 |
| 60 | 160.0 | 1735.0 | 3.0 |
| 75 | 192.6 | 2053.6 | 3.0 |
| 100 | 300.8 | 3147.3 | 4.0 |
| 110 | 268.8 | 2796.7 | 4.0 |
| 112 | 273.2 | 2839.2 | 4.0 |
| 120 | 290.6 | 3009.1 | 4.0 |
| 150 | 355.9 | 3646.1 | 4.0 |
| 180 | 421.2 | 4283.1 | 3.5 |
| 200 | 442.9 | 4471.7 | 3.5 |
| 240 | 551.8 | 5557.2 | 3.5 |
| 300 | 1054.0 | 8271.0 | 3.5 |

### DR Formulation

Linear calculated pharmacokinetic parameters for the delayed release (DR) formulation (prepared as described in Example 3, Table 18) are shown in Table 55.

TABLE 55
Linear Calculated Pharmacokinetic Parameters for DR Formulation

| | Mean Cₘₐₓ | Mean AUC_{0-∞} | Mean Tₘₐₓ |
|---|---|---|---|
| Dose (mg) | (ng/mL) | (ng/mL * h) | (hrs) |
| 12.5 | 27.2 | 265.4 | 4.7 |
| 14 | 30.5 | 297.2 | 4.7 |
| 15 | 32.7 | 318.5 | 4.7 |
| 25 | 54.4 | 530.8 | 4.7 |
| 28 | 61.0 | 594.4 | 4.7 |
| 30 | 65.3 | 636.9 | 4.7 |
| 45 | 127.3 | 1416.8 | 4.7 |
| 50 | 129.8 | 1449.0 | 4.7 |
| 56 | 151.3 | 1650.3 | 4.7 |
| 60 | 160.0 | 1735.2 | 4.7 |
| 75 | 192.6 | 2053.7 | 4.7 |
| 100 | 247.0 | 2584.4 | 4.7 |
| 110 | 268.8 | 2796.7 | 4.7 |
| 112 | 273.2 | 2839.2 | 4.7 |
| 120 | 290.6 | 3009.0 | 4.7 |
| 150 | 355.9 | 3645.9 | 4.7 |
| 180 | 421.2 | 4282.8 | 4.7 |
| 200 | 464.7 | 4707.4 | 4.7 |
| 240 | 551.8 | 5556.6 | 4.7 |
| 300 | 682.4 | 6830.4 | 4.7 |

### ER1Formulation

Linear calculated pharmacokinetic parameters for the extended release formulation prepared as described in Example 5, Table 35 (“ER1 formulation”), are shown in Table 56.

TABLE 56
Linear Calculated Pharmacokinetic Parameters for ER1 Formulation

| | Mean Cₘₐₓ | Mean AUC_{0-∞} | Mean Tₘₐₓ |
|---|---|---|---|
| Dose (mg) | (ng/mL) | (ng/mL * h) | (hrs) |
| 12.5 | 17.0 | 265.4 | 5.2 |
| 14 | 19.0 | 297.2 | 5.2 |
| 15 | 20.4 | 318.5 | 5.2 |
| 25 | 44.4 | 530.8 | 5.2 |
| 28 | 47.7 | 594.4 | 5.2 |
| 30 | 50.0 | 636.9 | 5.2 |
| 45 | 67.7 | 1416.4 | 5.2 |
| 50 | 88.3 | 1522.5 | 5.2 |
| 56 | 103.8 | 1550.0 | 5.3 |
| 60 | 100.1 | 1728.0 | 5.1 |
| 75 | 118.0 | 2053.3 | 5.2 |
| 100 | 147.0 | 2584.0 | 5.2 |
| 110 | 159.0 | 2796.3 | 5.2 |
| 112 | 162.0 | 2838.8 | 5.2 |
| 120 | 172.0 | 3008.6 | 5.2 |
| 150 | 207.0 | 3645.5 | 5.2 |
| 180 | 242.0 | 4282.4 | 5.2 |
| 200 | 265.0 | 4707.0 | 5.2 |
| 240 | 312.0 | 5556.2 | 5.2 |
| 300 | 383.0 | 6830.0 | 5.2 |

### ER2Formulation

Linear calculated pharmacokinetic parameters for the extended release formulation prepared as described in Example 6, Table 44 (“ER2Formulation”) are shown in Table 57.

TABLE 57
Linear Calculated Pharmacokinetic Parameters for ER2 Formulation

| | Mean Cₘₐₓ | Mean AUC_{0-∞} | Mean Tₘₐₓ |
|---|---|---|---|
| Dose (mg) | (ng/mL) | (ng/mL * h) | (hrs) |
| 12.5 | 11.4 | 241.5 | 6.6 |
| 14 | 12.8 | 270.5 | 6.6 |
| 15 | 13.7 | 289.8 | 6.6 |
| 25 | 25.5 | 634.8 | 6.6 |
| 28 | 25.6 | 797.2 | 6.6 |
| 30 | 27.4 | 761.7 | 6.6 |
| 45 | 53.5 | 1289.0 | 6.6 |
| 50 | 54.3 | 1357.8 | 6.6 |
| 56 | 63.5 | 1501.6 | 6.6 |
| 60 | 66.9 | 1574.0 | 6.6 |
| 75 | 80.9 | 1868.7 | 6.6 |
| 100 | 126.3 | 2864.0 | 6.6 |
| 110 | 112.9 | 2545.0 | 6.6 |
| 112 | 114.7 | 2583.7 | 6.6 |
| 120 | 122.0 | 2738.3 | 6.6 |
| 150 | 149.5 | 3318.0 | 6.6 |
| 180 | 176.9 | 3897.6 | 6.6 |
| 200 | 186.0 | 4069.2 | 6.6 |
| 240 | 231.8 | 5057.0 | 6.6 |
| 300 | 442.7 | 7526.6 | 6.6 |

One skilled in the art with the benefit of this disclosure may readily determine pharmacokinetic parameters for any specific dosage of milnacipran hydrochloride used in a formulation described herein.

### Example 9

### Conversion to Cyclic Degradation Product

Milnacipran hydrochloride was exposed to 0.1 M pH 9.0 buffer (glycine-NaOH) at 50° C. in a closed glass flask for several days. The amount of cyclic degradation product formed in the milnacipran hydrochloride is set forth in Table 58.

TABLE 58
Formation of Cyclic Degradation Product at 50° C.

| | Amount of | | |
|---|---|---|---|
| | Cyclic Degradation | | |
| | Product F1612 (% w/w) | | |
| Active Ingredient | 1 Day | 2 Days | 8 Days |
| Milnacipran | 0.11 | 0.23 | 0.90 |

As can be seen from Table 58, rapid formation of the cyclic degradation product occurs in milnacipran hydrochloride at 50° C. and pH 9.0.

### Example 10

### Milnacipran Formulations with Reduced Levels of Cyclic Degradation Product

Use of Low pH (pH 4-7) Polymer Coating System

Extended release formulations containing milnacipran hydrochloride was prepared according to Tables 59-61.

TABLE 59
Milnacipran HCl Extended Release Beads, 300 mg/g and 310 mg/g

| Ingredients | 300 mg/g | 310 mg/g |
|---|---|---|
| Sugar Spheres, USP (30-35 Mesh) | 315.0 | 326.7 |
| Milnacipran HCl | 300.0 | 310.0 |
| Polyethylene Glycol, NF (PEG 8000) | 99.8 | 103.3 |
| Polyethylene Glycol, NF (PEG 400) | 5.2 | 5.0 |
| HPMC-based film coating system (Opadry ®) | 18.0 | 18.3 |
| Talc, USP | 12.0 | 11.7 |
| Ethylcellulose aqueous dispersion | 180.0 | 155.0 |
| (Aquacoat ®, pH 4-7)¹ | | |
| Triethyl Citrate, PG/NF¹ | 45.0 | 38.3 |
| HPMC-based film coating system (Opadry ®)² | 25.0 | 31.7 |
| Purified Water, USP³ | — | — |
| TOTAL | 1000 | 1000 |

¹Amount indicated is as dry polymer.
²Optional top coating
³Purified water is removed during manufacturing process.

TABLE 60
Milnacipran HCl Extended Release Capsules, 56 mg

| | Theoretical Weight |
|---|---|
| Ingredients | (mg/capsule) |
| Weight of HPMC (hypromellose) | 75 mg (Size 1) |
| Capsule Shell | |
| Weight of Milnacipran HCl ER Beads, | 187 mg |
| 300 mg/g | |
| TOTALS Gross Weight | 262 mg/capsule |

TABLE 61
Milnacipran HCl Extended Release Capsules, 14 mg, 28 mg, 56 mg and 112 mg

| | Theoretical | Theoretical | Theoretical | Theoretical |
|---|---|---|---|---|
| | Weight | Weight | Weight | Weight |
| Ingredients | (mg/capsule) | (mg/capsule) | (mg/capsule) | (mg/capsule) |
| ER Capsule Strength | 14 | 28 | 56 | 112 |
| Weight of HPMC | 46 mg | 46 mg | 75 mg | 110 mg |
| (hypromellose) Capsule Shell | (size 3) | (size 3) | (size 1) | (size 0E) |
| Weight of Milnacipran HCl | 45 mg | 90 mg | 181 mg | 361 mg |
| Beads, 310 mg/g | | | | |
| Total Gross Weight | 91 mg/capsule | 136 mg/capsule | 256 mg/capsule | 471 mg/capsule |

Preparation of Milnacipran HCl ER Beads, 300 mg/g and 310 mg/g (Composition in Table 59)

Sugar spheres, USP (30-35 mesh) were coated in a fluid bed with a pre-prepared drug layering dispersion of milnacipran hydrochloride, polyethylene glycol, NF (PEG 8000), polyethylene glycol, NF (PEG 400), hydroxypropyl methylcellulose (Opadry®) and talc, USP in purified water, USP. The beads were dried, discharged from the fluid bed, and sieved to produce the milnacipran hydrochloride immediate release beads. The IR beads were coated with a layer of ethylcellulose (Aquacoat™, pH 4-7) and triethyl citrate in purified water, USP, followed by a layer of hydroxypropyl methylcellulose (Opadry®) in purified water, USP. The beads were then dried and cured in the fluid bed. After being discharged from the fluid bed, the thus formed extended release beads were sieved to obtain the final extended release bead product shown in Table 59.

Preparation of Milnacipran HCl ER Capsules, 14 mg, 28 mg, 56 mg and 112 mg/g (Composition in Tables 60 and 61)

Using an encapsulation machine fitted with one pellet dosing station, size 3, size 1 or size 0E capsules were filled with the milnacipran hydrochloride extended release beads (300 mg/g or 310 mg/g) described in Table 59 to the appropriate fill weight to afford the milnacipran HCl extended release capsules shown in Tables 60 and 61.

The dissolution rate for the 56 mg extended release capsules of Table 60 is shown in Table 62.

TABLE 62
Dissolution Rate for Extended Release Capsules

| Time (hr) | % Dissolved |
|---|---|
| 1 | 29 |
| 2 | 54 |
| 4 | 82 |
| 6 | 95 |
| 8 | 101 |
| 10 | 104 |
| 12 | 105 |

Stability data of extended release capsules described in Table 60 under different conditions is shown in Table 63. Condition A is 60 capsules in a 75 cc HDPE bottle, induction sealed, without desiccant. Condition B is the same as Condition A, but with desiccant.

TABLE 63
56 mg extended Release Capsules at 40° C./75% RH

| | Condition A | | Condition B | |
|---|---|---|---|---|
| | (without desiccant) | | (with desiccant) | |
| | Amount of F1612 | % | Amount of F1612 | % |
| Time | (% w/w) | Assay | (% w/w) | Assay |
| Initial | <0.05 | 103.2 | <0.05 | 103.2 |
| 1 Month | 0.06 | 102.4 | 0.06 | ND |
| 2 Months | 0.09 | 100.8 | 0.09 | ND |
| 3 Months | 0.12 | 102.2 | 0.09 | ND |
| 6 Months | 0.22 | 101.1 | 0.22 | ND |

ND: Not Determined

A comparison study for ethylcellulose dispersion system, pH 4-7 vs. pH ˜10, was performed. Extended release beads and capsules were prepared as per Table 46 and as described above procedures for ER beads 300 mg/g and 310 mg/g (Table 59).

TABLE 64
Milnacipran HCl Extended Release Beads, 289 mg/g

| | Dispersion | Dispersion |
|---|---|---|
| | pH 4-7 | pH ~10 |
| Ingredients | (Aquacoat ®) | (Surelease ®) |
| Sugar Spheres, USP (30-35 Mesh) | 350.0 | 350.0 |
| Milnacipran HCl | 289.0 | 289.0 |
| Polyethylene Glycol, NF (PEG 8000) | 68.6 | 68.6 |
| Polyethylene Glycol, NF (PEG 400) | 3.6 | 3.6 |
| Hydroxypropyl methylcellulose | 6.9 | 6.9 |
| (Opadry ®) | | |
| Talc, USP | 4.6 | 4.6 |
| Ethylcellulose aqueous dispersion | 202.4 | — |
| (Aquacoat ®, pH 4-7)¹ | | |
| Triethyl Citrate, PG/NF¹ | 50.6 | — |
| Ethylcellulose aqueous dispersion | — | 253.0 |
| (Surelease ®, pH ~10)¹ | | |
| Hydroxypropyl methylcellulose | 24.3 | 24.3 |
| (Opadry ®)² | | |
| Purified Water, USP³ | — | — |
| TOTAL | 1000 | 1000 |

¹Amount indicated is as dry polymer.
²Optional top coating
³Purified water is removed during the process.

TABLE 65
Milnacipran HCl Extended Release Capsules, 60 mg

| | Theoretical Weight |
|---|---|
| Ingredients | (mg/capsule) |
| Weight of Gelatin Capsule Shell | 76 mg |
| | (Size 1) |
| Weight of Milnacipran HCl ER Beads, | 208 mg |
| 289 mg/g | |
| TOTAL | 284 mg/capsule |

The effect of different pH conditions of ethylcellulose dispersion on the stability of the extended release capsules described in Table 65 is shown in Table 66. The bottle package contained 30 gelatin capsules in a 45 cc HDPE bottle, induction sealed, and without desiccant.

Surprisingly and unexpectedly, the formulation using pH 4-7 ethylcellulose dispersion greatly reduced the cyclic degradation product F1612, compared to the formulation using pH ˜10 ethylcellulose dispersion (Table 66).

TABLE 66
Milnacipran HCl Extended Release Capsules, 60 mg, prepared with
Ethylcellulose Dispersions under two pH Conditions (pH 4-7 and pH ~10)

| | Capsule filled with ER beads 289 mg/g | | | |
|---|---|---|---|---|
| | | | EC Dispersion | |
| | | | pH ~10 | |
| | EC Dispersion pH 4-7 | | (Surelease ®) | |
| | (Aquacoat ®) | | Amount of | |
| | Amount of F1612 | % | F1612 | % |
| Time | (% w/w) | Assay | (% w/w) | Assay |
| Initial | <0.05 | 106.6 | <0.05 | 97.4 |
| 40° C./75% RH 1 mo | 0.11 | 104.9 | 0.24 | 96.2 |
| 40° C./75% RH 2 mo | 0.19 | ND | 0.45 | ND |
| 30° C./65% RH 12 mo | 0.20 | 101.5 | 0.54 | 92.8 |

### Use of HPMC (Hypromellose) Capsules

A study using different capsule shell materials was performed. The water content in gelatin capsule shell was known to be 13-16% and in HPMC capsule shell 4-6%. In the study, the previously prepared ER beads of 289 mg/g (pH 4-7 ethylcellulose dispersion) and 323 mg/g (pH ˜10 ethylcellulose dispersion), per compositions in Tables 34 and 64, were used. Each ER bead batch was encapsulated into 60 mg capsules with two types of capsule shells, and put on bottle stability study. ER bead formulation of 289 mg/g is shown in Table 64. ER beads of 323 mg/g are listed in Table 34. 60 counts of ER capsules are placed 75 cc bottle without desiccant.

Surprisingly and unexpectedly, compared to gelatin capsules the HPMC capsules which contains lower water level was found to play a significant role in reducing moisture-induced cyclic formation of F1612 (Table 67). Extended release beads and capsules were prepared as per formulation for described above.

TABLE 67
Milnacipran HCl Extended Release Capsules,
comparing capsule shell material
(HPMC Capsule vs. Gelatin Capsule)

| | Capsule material | | | |
|---|---|---|---|---|
| | | | Capsules filled | |
| | | | with ER beads | |
| | Capsules filled with | | 323 mg/g | |
| | ER beads | | (EC Dispersion | |
| | 289 mg/g (EC | | pH ~10, | |
| | Dispersion pH 4-7, | | Surelease ®) | |
| | Aquacoat ®) | | Gelatin | HPMC |
| | Gelatin shell | HPMC shell | shell | shell |
| Initial | <0.05 | <0.05 | <0.05 | <0.05 |
| 40° C./75% RH × 2 M | 0.19 | 0.13 | 0.28 | 0.13 |

### Use of Organic Solvent

To evaluate the effect of aqueous coating and solvent coating on degradation level, extended release formulations were prepared using ethanol as the coating solvent, as described below in Tables 68-75.

TABLE 68
Milnacipran HCl IR Beads, 400 mg/g and 600 mg/g
(Organic Solvent Process)

| | 400 mg/g | | 600 mg/g | |
|---|---|---|---|---|
| Sugar Spheres, USP/NF (30-35 Mesh) | 550.0 | | 325.0 | |
| Milnacipran HCl | 400.0 | | 600.0 | |
| Povidone, USP | 20.0 | | 30.0 | |
| Talc, USP | 30.0 | | 45.0 | |
| Ethanol denatured* | — | | — | |
| TOTAL (IR Beads) | 1000 | mg | 1000 | mg |

*Ethanol (denatured) is removed during the process.

TABLE 69
Milnacipran HCl ER Beads, 370 mg/g (Organic Solvent Process)

| | Theoretical | |
|---|---|---|
| Ingredients | Weight, mg/g | |
| Milnacipran HCl IR Beads, 400 mg/g | 926.0 | |
| (solvent process) | | |
| Ethylcellulose, NF (20 cp)¹ | 60.7 | |
| Triethyl Citrate, NF¹ | 12.1 | |
| Talc, USP¹ | 1.2 | |
| Ethanol (denatured)^{1, 2} | — | |
| TOTAL | 1000 | mg |

¹These component were added in the form of a polymer dispersion consisting of Ethocel, Triethyl Citrate, Talc, and Ethanol.
²Ethanol (denatured) is removed during the process.

TABLE 70
Milnacipran HCl ER Beads, 372 mg/g (Organic Solvent Process)

| | Theoretical | |
|---|---|---|
| Ingredients | Weight, mg/g | |
| Milnacipran HCl IR Beads, 400 mg/g | 930.2 | |
| Ethylcellulose, NF (45 cp)¹ | 55.4 | |
| Triethyl Citrate, NF¹ | 11.1 | |
| Talc, USP¹ | 3.3 | |
| Ethanol (denatured)^{1, 2} | — | |
| TOTAL | 1000 | mg |

¹These component were added in the form of a polymer dispersion consisting of Ethocel, Triethyl Citrate, Talc, and Ethanol.
²Ethanol (denatured) is removed during the process.

TABLE 71
Milnacipran HCl ER Beads, 333 mg/g (Organic Solvent Process)

| | Theoretical |
|---|---|
| Ingredients | Weight, mg/g |
| Milnacipran HCl IR Beads, 400 mg/g | 833.3 |
| Ethylcellulose, NF (10 cp)¹ | 138.9 |
| Triethyl Citrate, NF¹ | 27.8 |
| Purified Water, USP^{1, 2} | — |
| Isopropyl alcohol^{1, 2} | — |
| TOTAL | 1000.0 |

¹These component were added in the form of a polymer dispersion consisting of Ethocel, Triethyl Citrate, Talc, and Ethanol.
²The solvent, isopropyl alcohol mixed with water in 9:1 w/w ratio, is removed during the process.

TABLE 72
Milnacipran HCl ER Beads, 504 mg/g and 509 mg/g
(Organic Solvent Process)

| ER bead strength | 504 mg/g | | 509 mg/g | |
|---|---|---|---|---|
| Milnacipran HCl Beads, 600 mg/g | 840 | | 848 | |
| (Solvent) | | | | |
| Ethylcellulose, NF (45 cp) | 114 | | 108 | |
| Triethyl Citrate, PG/NF | 23 | | 22 | |
| Talc, USP | 23 | | 22 | |
| Ethanol (unsaturated) | — | | — | |
| TOTAL | 1000 | mg | 1000 | mg |

TABLE 73
Milnacipran HCl ER Capsules, 60 mg (Organic Solvent Process)

| | Theoretical |
|---|---|
| | Weight, |
| Ingredients | mg/capsule |
| Weight of Milnacipran HCl ER | 162 mg |
| Beads, 370 mg/g (composition in | |
| Table 69) | |
| Weight of HPMC Capsules | 75 mg (Size 1) |
| TOTAL Gross Weights | 237 mg/capsule |

TABLE 74
Milnacipran HCl ER Capsules, 60 mg (Organic Solvent Process)

| | Theoretical Weight, |
|---|---|
| Ingredients | mg/capsule |
| Weight of Milnacipran HCl ER | 161 mg |
| Beads, 372 mg/g (composition in | |
| Table 70) | |
| Weight of HPMC Capsules | 75 mg (Size 1) |
| TOTAL Gross Weights | 236 mg/capsule |

TABLE 75
Milnacipran HCl ER Capsules, 60 mg (Organic Solvent Process)

| | Theoretical Weight, |
|---|---|
| Ingredients | mg/capsule |
| Weight of Milnacipran HCl ER | 180 mg |
| Beads, 333 mg/g (composition in | |
| Table 71) | |
| Weight of Gelatin Capsules | 76 mg (Size 1) |
| TOTAL Gross Weights | 256 mg/capsule |

TABLE 76
Milnacipran HCl ER Capsules, 112 mg (Organic solvent process)

| | Theoretical |
|---|---|
| | Weight, |
| Ingredients | mg/capsule |
| Weight of Milnacipran HCl ER | 220 mg |
| Beads, 509 mg/g (mg) | |
| Weight of HPMC capsule, size 0E | 110 mg |
| (mg) | |
| TOTAL Gross Weights | 330 mg/capsule |

Preparation of Milnacipran HCl IR Beads, 400 mg/g and 600 mg/g (Organic Solvent Process, Composition in Table 68)

Sugar spheres, USP (30-35 mesh) were coated in a fluid bed with a pre-prepared drug layering dispersion of milnacipran hydrochloride, povidone USP, and talc, USP in denatured ethanol. The beads were dried and then discharged from the fluid bed. The discharged beads were then sieved to produce the milnacipran hydrochloride IR beads, 370 mg/g or 600 mg/g.

Preparation of Milnacipran HCl ER Beads, 370 mg/g, 372 mg/g, 333 mg/g, 504 mg/g or 509 mg/g (Organic Solvent Process, Composition in Table 69, 70, 71 or 72)

The milnacipran hydrochloride IR beads described above were coated with a solvent dispersion consisting of ethylcellulose, triethyl citrate, talc, and ethanol. The beads were then dried and cured in the fluid bed. After being discharged from the fluid bed, the thus formed extended release beads were sieved to obtain the final extended release bead product shown in Tables 69, 70, 71 and 72.

### Preparation of Milnacipran HCl ER Capsules, 60 mg or 112 mg (Composition in Tables 73-76)

Using an encapsulation machine fitted with one pellet dosing station, size 1 or size 0E capsules were filled with the milnacipran hydrochloride extended release beads (370 mg/g, 372 mg/g, 333 mg/g, 504 mg/g or 509 mg/g) described in Tables 69-73 to the appropriate fill weight to afford the milnacipran HCl extended release capsules shown in Tables 73-76.

The dissolution rate for the extended release beads of Table 69 is shown in Table 77.

TABLE 77
Dissolution Rate for Extended Release Beads, 370 mg/g

| Time (hr) | % Dissolved |
|---|---|
| 1 | 48 |
| 2 | 67 |
| 4 | 83 |
| 6 | 90 |
| 8 | 96 |
| 10 | 98 |
| 12 | 101 |

The dissolution rate for the extended release beads of Table 71 is shown in Table 78.

TABLE 78
Dissolution Rate for Extended Release Beads, 333 mg/g

| Time (hr) | % Dissolved |
|---|---|
| 1 | 28 |
| 2 | 48 |
| 4 | 68 |
| 6 | 80 |
| 8 | 88 |
| 10 | 93 |
| 12 | 96 |

The dissolution rate for the extended release capsules of Table 74 is shown in Table 79.

TABLE 79
Dissolution Rate for Extended Release Capsules

| Time (hr) | % Dissolved |
|---|---|
| 1 | 28 |
| 2 | 56 |
| 4 | 76 |
| 6 | 84 |
| 8 | 90 |
| 10 | 93 |
| 12 | 96 |

The concentration of milnacipran (illustrated in “assay” column) as well as the cyclic degradation product F1612 in the extended release capsules of Table 73 was assayed over a 6 month period (at 40° C./75% RH), and is illustrated in Table 80. Condition A is 50 capsules in a 75 cc HDPE bottle, induction sealed, without desiccant. Condition B is 50 capsules in a 75 cc HDPE bottle, induction sealed, with desiccant.

TABLE 80
Extended Release Capsules at 40° C./75% RH

| | Condition A | | Condition B | |
|---|---|---|---|---|
| | Amount of F1612 | % | Amount of F1612 | % |
| Time | (% w/w) | Assay | (% w/w) | Assay |
| Initial | <0.05 | 101.2 | <0.05 | 101.2 |
| 1 Month | <0.05 | 102.3 | <0.05 | 102.4 |
| 3 Months | <0.05 | 102.7 | 0.06 | 101.9 |
| 6 Months | 0.08 | 101.8 | 0.08 | 102.1 |

The concentration of milnacipran (illustrated in “assay” column) as well as the degradation product F1612 in the extended release capsules of Table 74 were tested over a 6 month period (at 40° C./75% RH), and is illustrated in Table 81. Condition A is 50 capsules in a 75 cc HDPE bottle, induction sealed, without desiccant. Condition B is 50 capsules in a 75 cc HDPE bottle, induction sealed, with desiccant.

TABLE 81
Extended Release Capsules at 40° C./75% RH

| | Condition A | | Condition B | |
|---|---|---|---|---|
| | Amount of F1612 | % | Amount of F1612 | % |
| Time | (% w/w) | Assay | (% w/w) | Assay |
| Initial | <0.05 | 100.1 | <0.05 | 100.1 |
| 1 Month | <0.05 | 100.4 | <0.05 | 100.6 |
| 3 Months | <0.05 | 101.4 | <0.05 | 101.0 |
| 6 Months | 0.06 | 100.5 | 0.06 | 101.0 |

The concentration of milnacipran (illustrated in “assay” column) as well as the cyclic degradation product F1612 in the extended release capsules of Table 75 was assayed over a 6 month period (at 40° C./75% RH), and is illustrated in Table 82.

TABLE 82
Extended Release Capsules at 40° C./75% RH

| | Amount of F1612 | % |
|---|---|---|
| Time | (% w/w) | Assay |
| Initial | <0.05 | 98.7 |
| 1 Month | 0.06 | 97.0 |
| 2 Months | 0.11 | ND |
| 3 Months | 0.17 | ND |

Under solvent coating, moisture level is kept very low. Thus cyclic formulation of degradation product F1612 is also significantly reduced.

### Example 11

### A Single-Center, Randomized, Open-Label, Two-Way Crossover, Two Sequence, Multiple Escalating Dose Study in Healthy Male and Female Subjects

A single-center, randomized, open-label, two-way crossover, two sequence, multiple escalating dose Study in healthy male and female subjects was conducted. The primary objective was to evaluate the bioequivalence between a 112 mg extended release bead formulation of the present invention and a 100 mg immediate release (IR) tablet of milnacipran hydrochloride.

Forty healthy male and female subjects (18-45 years of age) were randomized to receive each of the following treatments, in one of two sequences, separated by a 7-day washout period.

Treatment A

Day 1: One 12.5 mg milnacipran hydrochloride IR tablet at 2000 hours;

Days 2-3: One 12.5 mg milnacipran hydrochloride IR tablet at 0800 and 2000 hours;

Days 4-7: One 25 mg milnacipran hydrochloride IR tablet at 0800 and 2000 hours;

Days 8-11: One 50 mg milnacipran hydrochloride IR tablet at 0800 and 2000 hours;

Days 12-14: One 100 mg milnacipran hydrochloride IR tablet at 0800 and 2000 hours;

Day 15: One 100 mg milnacipran hydrochloride IR tablet at 0800 hours;

The IR tablet formulations were prepared as described in Table 8 of Example 2.

Treatment B

Day 1: One 14 mg milnacipran hydrochloride capsule at 2000 hours;

Days 2-3: One 14 mg milnacipran hydrochloride capsule at 0800 and 2000 hours;

Days 4-7: One 28 mg milnacipran hydrochloride capsule at 0800 and 2000 hours;

Days 8-11: One 56 mg milnacipran hydrochloride capsule at 0800 and 2000 hours;

Days 12-14: One 112 mg milnacipran hydrochloride capsule at 0800 and 2000 hours;

Day 15: One 112 mg milnacipran hydrochloride capsule at 0800 hours.

The extended release capsule formulations were prepared as described in Table 61.

Sequence I: Treatment A followed by Treatment B

Sequence II: Treatment B followed by Treatment A.

The total duration of the study was 38 days (Day 1 through last PK blood sample collection on Day 38), not including the screening visit. 30 patients completed the study.

The mean pharmacokinetic parameters observed during the study are shown in Table 83.

TABLE 83
Mean PK Parameters

| | IR Tablet | Bead | Ratio | |
|---|---|---|---|---|
| | Formulation | Formulation* | (56 mg beads/50 mg IR | |
| Parameter | (n = 30) | (n = 30) | tablets) % | 90% CI (%) |
| Cₘₐₓ | 453.6 (29.8) | 382.5 (29.0) | 84.3 | 75.0-94.9 |
| (ng/mL) | | | | |
| AUCₜₐᵤ | 3031.1 (26.9) | 3271.2 (32.3) | 107.9 | 96.4-120.8 |
| (ng · h/mL) | | | | |
| Cₘᵢₙ | 112.0 (28.7) | 168.6 (38.9) | 150.5 | 134.5-168.4 |
| (ng/mL) | | | | |
| Tₘₐₓ | 2.0 (0.0, 3.0) | 3.0 (9.0, 5.0) | — | — |
| (h) | | | | |
| T_{1/2} | 8.3 (1.7) | 9.7 (1.7) | — | — |
| (h) | | | | |

*Bead formulation prepared as described in tables 59-61.

### Example 12

### Milnacipran Formulations with High Levels of Drug Layering, Prepared Through Use of Powder Layering Technique

Preparation of Milnacipran HCl IR Beads, 750 mg/g

Sugar spheres, USP were charged into a rotor processor Granurex GX-35 (Vector Corp.). Finely milled milnacipran hydrochloride and a binder solution of hydroxypropyl methylcellulose (Opadry®) in purified water, USP were added simultaneously into the bed of sugar spheres, each at a controlled rate. The beads were dried in the rotor processor. The discharged beads were then sieved to produce the milnacipran hydrochloride immediate release beads, 750 mg/g. The formulations of high drug layered IR beads are shown in Table 84.

### Preparation of Milnacipran HCl ER Beads

The milnacipran hydrochloride IR beads described above may be coated with a dispersion containing ethylcellulose aqueous dispersion (Aquacoat®, pH 4-7) and triethyle citrate in purified water, USP, to a target weight gain followed by a layer of hydroxypropyl methylcellulose (Opadry®) in purified water, USP. The beads are then dried, cured, and sieved to obtain the final extended release bead product.

TABLE 84
Milnacipran HCl Immediate Release Beads

| | | | Preferred | | |
|---|---|---|---|---|---|
| | Range 1 | Range 2 | Range | Example 1 | Example 2 |
| Ingredient | mg/g | mg/g | mg/g | mg/g | mg/g |
| Sugar Spheres, | 100-900 | 160-800 | 275-500 | 334.4 | 227.5 |
| USP | | | | | |
| Milnacipran HCl | 100-850 | 200-800 | 500-700 | 650 | 750 |
| Hydroxypropyl | 2-40 | 4-38 | 9-34 | 15.6 | 22.5 |
| methylcellulose | | | | | |
| (Opadry ®) | | | | | |
| Talc, USP | 0-17 | 0-16 | 0-14 | 0 | 0 |
| Purified Water, | | — | — | — | — |
| USP* | | | | | |
| Total mg | 1000 | 1000 | 1000 | 1000 | 1000 |

*Purified Water is removed during the manufacturing process.

### Example 13

### Milnacipran HCl Delayed Release (DR) Tablets with Polyvinyl Alcohol (PVA) Subcoat

Milnacipran HCl DR tablets may be prepared in the following manner. Polyvinyl pyrrolidone (PVP) is dissolved in purified water with stirring until a clear solution is obtained. Milnacipran HCl, dicalcium phosphate powder and calcium carboxymethyl cellulose are added to the bowl of a high shear granulator and mixed till uniform. The PVP solution is then added to the granulator under mixing and chopping till the formation of fine and uniform granules. The granules are dried in a fluid bed followed by milling with a Quadro Comil to the desired granule particle size. The granules are then blended with calcium carboxymethyl cellulose, colloidal silica dioxide and talc. The blend is compressed dose-proportionally into tablets of four strengths: 12.5, 25, 50 and 100 mg. The IR tablet obtained is then coated with polyvinyl alcohol (PVA) as the subcoat followed by coating with a DR coat to form a DR tablet.

Table 85 illustrates proposed milnacipran HCl DR tablets produced according to the method described above.

TABLE 85
Proposed Composition of milnacipran HCl DR tablets

| | Tablet Potency | | | |
|---|---|---|---|---|
| Ingredient | 12.5 mg | 25 mg | 50 mg | 100 mg |
| Milnacipran HCl | 12.50 | 25.00 | 50.00 | 100.00 |
| Dicalcium phosphate dihydrate, | 5.78 | 11.56 | 23.12 | 46.24 |
| USP (milled, Calipharm ® D) | | | | |
| Dicalcium phosphate dihydrate, | 22.30 | 44.60 | 89.20 | 178.40 |
| USP (unmilled, Di-Tab ®) | | | | |
| Calcium carboxymethyl cellulose, | 11.62 | 23.24 | 46.48 | 92.96 |
| NF | | | | |
| Povidone, USP | 1.10 | 2.20 | 4.40 | 8.80 |
| Colloidal silica dioxide, NF, EP | 0.32 | 0.64 | 1.28 | 2.56 |
| Talc, USP | 1.10 | 2.20 | 4.40 | 8.80 |
| Magnesium stearate, NF, EP, BP | 0.28 | 0.56 | 1.12 | 2.24 |
| Core Tablet Weight | 55.0 | 110.0 | 220.0 | 440.0 |
| PVA coating | 1.65 | 3.30 | 6.60 | 13.20 |
| Methacrylic acid copolymer | 11.33 | 22.66 | 45.32 | 90.64 |
| TOTAL | 68.0 | 136.0 | 271.9 | 543.8 |

For DR formulations, the dissolution may be performed in two release media sequentially: 2 hrs in 0.1N HCl (acidic stage) followed by 1 hr in pH 6.8 buffer. As shown in Table 86, the expected dissolution profile of milnacipran DR tablet is 1) less than 10% drug released in pH 1.2; 2) immediate release in pH 6.8 buffer.

Table 86 provides the expected dissolution rate for milnacipran HCl delayed release tablets.

TABLE 86
Expected Dissolution Rate for Milnacipran HCl DR tablets

| Dissolution | | |
|---|---|---|
| Stage | Time (mins) | % Dissolved |
| pH 1.2 | 0 | 0 |
| | 60 | 0 |
| | 120 | 5 |
| pH 6.8 | 135 | 96 |
| | 150 | 98 |
| | 165 | 100 |

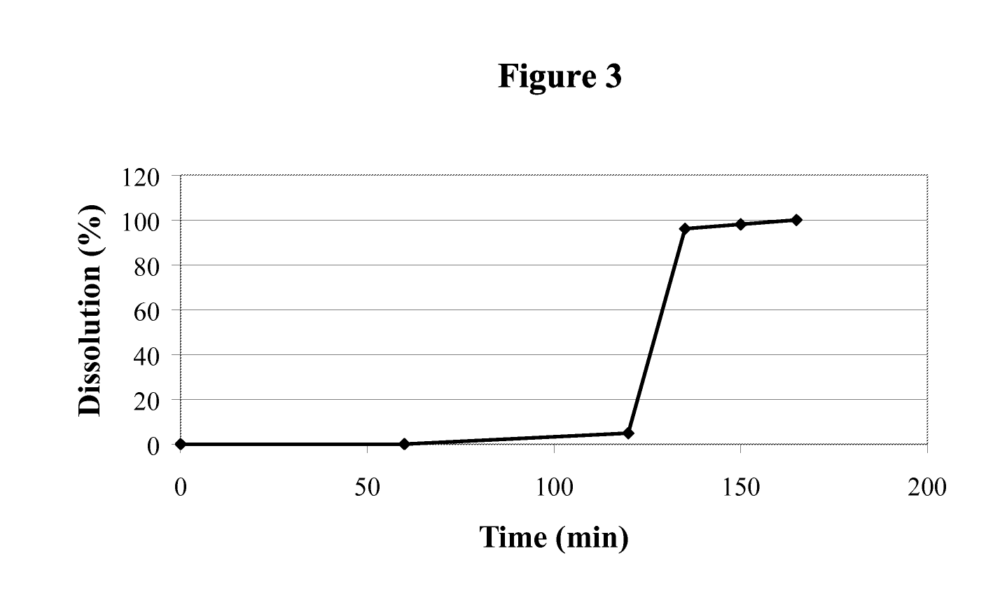

FIG. 3 shows an expected dissolution profile for milnacipran hydrochloride delayed release (DR) tablets.

Table 87 provides expected stability data for milnacipran HCl DR tablets.

TABLE 87
Expected stability data for milnacipran HCl DR tablets

| Strength | Time | Deg (F1612) |
|---|---|---|
| 12.5 mg | initial | <0.05 |
| | 40° C., 75% RH, 3 mo | 0.228 |
| 25 mg | initial | <0.05 |
| | 40° C., 75% RH, 3 mo | 0.2 |
| 50 mg | initial | <0.05 |
| | 40° C., 75% RH, 3 mo | 0.177 |
| 100 mg | initial | 0.057 |
| | 40° C., 75% RH, 3 mo | 0.218 |

### Example 14

### Milnacipran HCl Extended Release (ER) Tablets with Polyvinyl Alcohol (PVA) Subcoat

An extended release (ER) tablet formulation may be prepared that releases milnacipran over an extended period of time leading to lower peak plasma concentrations and/or to a prolonged Tₘₐₓ, as compared to an immediate release formulation of milnacipran. The milnacipran ER tablet discussed below may be made in three steps: preparing the milnacipran HCl IR tablets; applying PVA sub-coat to the core tablets and applying ER coating to the tablets with PVA sub-coat. The composition of proposed milnacipran HCl ER tablets is illustrated in Table 88.

TABLE 88
Composition of milnacipran HCl ER tablets

| | Tablet Potency | | | |
|---|---|---|---|---|
| Ingredient | 12.5 mg | 25 mg | 50 mg | 100 mg |
| Milnacipran HCl | 12.50 | 25.00 | 50.00 | 100.00 |
| Dicalcium phosphate dihydrate, | 5.78 | 11.56 | 23.12 | 46.24 |
| USP (milled, Calipharm ® D) | | | | |
| Dicalcium phosphate dihydrate, | 22.30 | 44.60 | 89.20 | 178.40 |
| USP(unmilled, Di-Tab ®) | | | | |
| Calcium carboxymethyl cellulose, | 11.62 | 23.24 | 46.48 | 92.96 |
| NF | | | | |
| Povidone, USP | 1.10 | 2.20 | 4.40 | 8.80 |
| Colloidal silica dioxide, NF, EP | 0.32 | 0.64 | 1.28 | 2.56 |
| Talc, USP | 1.10 | 2.20 | 4.40 | 8.80 |
| Magnesium stearate, NF, EP, BP | 0.28 | 0.56 | 1.12 | 2.24 |
| Core Tablet Weight | 55.0 | 110.0 | 220.0 | 440.0 |
| PVA coating, 3% wt gain | 1.65 | 3.30 | 6.60 | 13.20 |
| Ethyl cellulose, 8% wt gain | 11.33 | 22.66 | 45.32 | 90.64 |
| TOTAL | 68 | 119 | 271.9 | 543.8 |

For ER formulations, the dissolution is performed in dissolution medium of pH 6.8. The expected dissolution profile of milnacipran ER tablet is shown in Table 89.

TABLE 89
Dissolution of Milnacipran HCl ER tablets

| Time (hr) | % Dissolved |
|---|---|
| 1 | 40 |
| 2 | 60 |
| 4 | 85 |
| 6 | 95 |
| 8 | 99 |
| 10 | 100 |
| 12 | 102 |

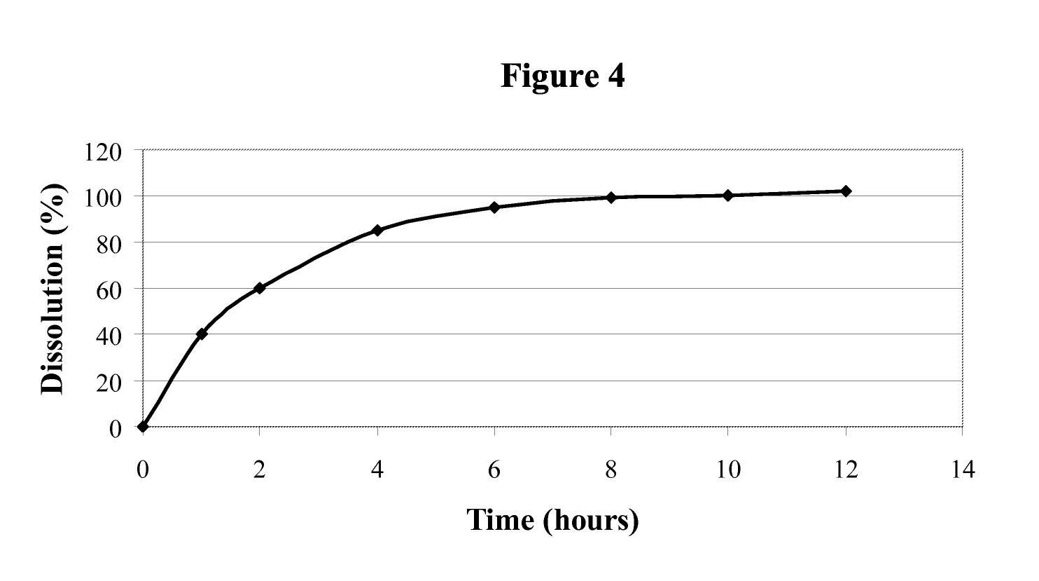

FIG. 4 shows an expected dissolution profile for milnacipran hydrochloride extended release (ER) tablets.

Table 90 provides the expected stability data for milnacipran HCl ER tablets.

TABLE 90
Expected stability of milnacipran HCl DR tablet

| Strength | Time | Deg (F1612) |
|---|---|---|
| 12.5 mg | initial | <0.05 |
| | 40° C., 75% RH, 3 m | 0.228 |
| 25 mg | initial | <0.05 |
| | 40° C., 75% RH, 3 m | 0.2 |
| 50 mg | initial | <0.05 |
| | 40° C., 75% RH, 3 m | 0.177 |
| 100 mg | initial | 0.057 |
| | 40° C., 75% RH, 3 m | 0.218 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

The entire disclosures of all applications, patents and publications, cited herein, are hereby incorporated by reference in their entirety.

1. A pharmaceutical formulation comprising: (i) about 12.5 mg to about 200 mg milnacipran or a pharmaceutically acceptable salt thereof and (ii) about 0.03 to about 0.5% w/w 1-phenyl-3-aza-bicyclo[3.1.0]hexan-2-one; wherein the formulation releases more than about 80% milnacipran or pharmaceutically acceptable salt thereof within 30 minutes upon entry in a use environment.

2. The formulation according to claim 1, wherein the formulation releases the milnacipran or pharmaceutically acceptable salt thereof in a single phase.

3. The formulation according to claim 1, wherein the formulation comprises milnacipran hydrochloride.

4. The formulation according to claim 1, wherein the formulation is a matrix.

5. The formulation according to claim 1, wherein the formulation is a bead.

6. The formulation according to claim 1, wherein the formulation is a tablet for oral administration.

7. The formulation according to claim 1, wherein the formulation is a capsule for oral administration.

8. The formulation according to claim 1, wherein the formulation is a tablet comprising about 12.5 mg milnacipran or pharmaceutically acceptable salt thereof.

9. The formulation according to claim 1, wherein the formulation is a tablet comprising about 25 mg milnacipran or pharmaceutically acceptable salt thereof.

10. The formulation according to claim 1, wherein the formulation is a tablet comprising about 50 mg milnacipran or pharmaceutically acceptable salt thereof.

11. The formulation according to claim 1, wherein the formulation is a tablet comprising about 100 mg milnacipran or pharmaceutically acceptable salt thereof.

12. The formulation according to claim 1, wherein the formulation comprises about 0.03 to about 0.5% 1-phenyl-3-aza-bicyclo[3.1.0]hexan-2-one after about 3 months at 40° C. and 75% relative humidity.

13. A pharmaceutical formulation comprising (i) about 12.5 mg to about 200 mg milnacipran or a pharmaceutically acceptable salt thereof; (ii) about 0.03 to about 0.5% w/w 1-phenyl-3-aza-bicyclo[3.1.0]hexan-2-one; and (iii) about 0.5 to about 8% w/w of a coating comprising a polymer in combination with a surfactant; wherein the coating has an adhesion force of more than about 25 N.

14. The formulation according to claim 13, wherein the said coating has an adhesion force of more than about 30 N.

15. The formulation according to claim 13, wherein the said coating has an adhesion force of between about 30 N and 35 N.

16. The formulation according to claim 13, wherein the said coating has an adhesion force of between about 35 N and about 40 N.

17. The formulation according to claim 13, wherein the polymer is selected from the group consisting of polyvinyl pyrrolidone, polyvinyl alcohol, hydroxypropyl cellulose, hydroxypropylmethyl cellulose, hydroxyethylmethyl cellulose, sodium carboxymethylcellulose, calcium carboxymethylcellulose, amylase starch, chitosan, hydroxyethyl cellulose, polydextrose, polyethylene oxide, maltodextrin and methyl cellulose.

18. The formulation according to claim 13, wherein the polymer is hydroxypropylmethyl cellulose.

19. The formulation according to claim 13, wherein the polymer is polyvinyl alcohol.

20. The formulation according to claim 13, wherein the surfactant is selected from the group consisting of polyethylene glycol, triacetin, glycerol, propylene glycol, acetyltributyl citrate, acetyltriethyl citrate, dibutyl phthalate, dibutyl sebacate, diethyl phthalate, glycerin, glycerin monostearate, tributyl citrate and triethyl citrate.

21. The formulation according to claim 13, wherein the surfactant is polyethylene glycol.

22. The formulation according to claim 13, wherein the formulation comprises about 12.5 mg milnacipran hydrochloride.

23. The formulation according to claim 13, wherein the formulation comprises about 25 mg milnacipran hydrochloride.

24. The formulation according to claim 13, wherein the formulation comprises about 50 mg milnacipran hydrochloride.

25. The formulation according to claim 13, wherein the formulation comprises about 100 mg milnacipran hydrochloride.

26. A pharmaceutical formulation comprising (i) about 12.5 mg to about 200 mg milnacipran or a pharmaceutically acceptable salt thereof; (ii) about 0.03 to about 0.5% w/w 1-phenyl-3-aza-bicyclo[3.1.0]hexan-2-one; and (iii) about 0.5 to about 8% w/w of a coating comprising a polymer in combination with a surfactant in a ratio of between about 1:1 and about 20:1.

27. The formulation according to claim 26, wherein the ratio of polymer to surfactant is between about 2:1 and 10:1.

28. The formulation according to claim 26, wherein the ratio of the polymer to the surfactant is selected from the group consisting of about 1, about 1.5, about 2, about 2.5, about 3.5, about 4, about 4.5, about 5, about 5.5, about 6, about 6.5, about 7, about 7.5, about 8, about 8.5, about 9, about 9.5, about 10, about 10.5, about 11, about 11.5 and about 12.

29. The formulation according to claim 26, wherein the formulation comprises about 12.5 mg milnacipran hydrochloride.

30. The formulation according to claim 26, wherein the formulation comprises about 25 mg milnacipran hydrochloride.

31. The formulation according to claim 26, wherein the formulation comprises about 50 mg milnacipran hydrochloride.

32. The formulation according to claim 26, wherein the formulation comprises about 100 mg milnacipran hydrochloride.

33-69. (canceled)
